(19) United States
(12) Patent Application Publication
(10) Pub. No.: US 2026/0286334 A1
(43) Pub. Date: Sep. 24, 2026

(54) BIOSYNTHESIS OF BETA-LACTAM ANTIBIOTICS

(71) Applicant: GINKGO BIOWORKS, INC., BOSTON, MA (US)

(72) Inventors: KRUPANANDAN HARANAHALLI, BOSTON, MA (US); SHURONG HOU, BOSTON, MA (US); JONATHAN LEWIS MCMURRY, BOSTON, MA (US); DYLAN STILES, BOSTON, MA (US); ABRAHAM WALDMAN, BOSTON, MA (US); EMILY E. WRENBECK, BOSTON, MA (US); MORTEN MARTINUS CORNELIS HARALD VAN SCHIE, CG DELFT (NL); THOMAS VAN DER DOES, CJ WILNIS (NL)

(21) Appl. No.: 19/473,826

(22) PCT Filed: Apr. 9, 2024
(86) PCT No.: PCT/US2024/023749

§ 371 Date: Oct. 9, 2025

Related U.S. Application Data
(60) Provisional application No. 63/458,398, filed on Apr. 10, 2023.

(51) Int. Cl. C12N 9/84; C12N 15/62; C12N 15/70; C12P 35/04; C12P 37/04; C12R 1/19

(52) U.S. Cl. CPC C12N 9/84; C12N 15/625; C12N 15/70; C12P 35/04; C12P 37/04; C07K 2319/02; C12R 2001/19; C12Y 305/01011

(57) ABSTRACT

Penicillin G acylase (PGA) enzymes, including engineered PGA enzymes, and their use in catalyzing chemical reactions related to biosynthesis of beta-lactam antibiotics.

### CROSS-REFERENCE TO RELATED APPLICATIONS

This application claims the benefit under 35 U.S.C. § 119 (e) of U.S. Provisional Application No. 63/458,398, filed Apr. 10, 2023, entitled “BIOSYNTHESIS OF BETA-LACTAM ANTIBIOTICS,” the entire disclosure of which is hereby incorporated by reference in its entirety.

### REFERENCE TO AN ELECTRONIC SEQUENCE LISTING

The contents of the electronic sequence listing (G0919.70100WO00-SEQ-KVC.xml; Size: 153,475 bytes; and Date of Creation: Apr. 8, 2024) is herein incorporated by reference in its entirety.

### FIELD

The present disclosure relates to the use of penicillin G acylase enzymes, including engineered penicillin G acylase enzymes, in the production of β-lactam antibiotics, such as D-amoxicillin and/or cephalexin.

### BACKGROUND

D-amoxicillin and cephalexin are examples of antibiotics that contain a β-lactam ring in their chemical structures (β-lactam antibiotics). β-lactam antibiotics are the most widely used group of antibiotics for the prevention and treatment of bacterial infections; however, the production of β-lactam antibiotics is costly and laborious. Currently, cost-effective and simple approaches to produce high quantities of β-lactam antibiotics remain elusive.

### SUMMARY

Aspects of the present disclosure relate to a penicillin G acylase (PGA), wherein the PGA comprises the following amino acid substitutions relative to the sequence of SEQ ID NO: 1: (i) F330A and W333I; (ii) M182I and F330A; (iii) T90V, A189V, F330A, W333I and D380V; (iv) T90V, A189V, F330A, D380V, H498L and V563Q; (v) M182L, F330A and T482P; (vi) T90V, A189V, F330A, W333I, D380I and H498L; (vii) M182T, F330A and L362F; (viii) M182L, R185F, F330A and T482P; (ix) F330G; (x) F330A and W333Y; (xi) R185F, N326A, F330A and T482P; (xii) F330A and W333P; (xiii) F330A and L770T; (xiv) T181M and F330A; (xv) F330A and D380Q; (xvi) M182N and F330A; (xvii) R185F, F330A and T482P; (xviii) F330A and L770A; (xix) T90V, A189V, F330A, W333T, D380V and H498S; or (xx) T181M, M182F, F330A and L362F.

In some embodiments, the PGA comprises the following amino acid substitutions relative to the sequence of SEQ ID NO: 1: (i) F330A and W333Y; (ii) T90V, A189V, F330A, W333I and D380V; (iii) T90V, A189V, F330A, D380V, H498L and V563Q; (iv) T90V, A189V, F330A, W333I, D380I and H498L; (v) F330G; (vi) F330A and W333P; (vii) F330A and L770T; (viii) F330A and D380Q; (ix) F330A and W333I; (x) F330A and L770A; or (xi) T90V, A189V, F330A, W333T, D380V and H498S. In some embodiments, the PGA comprises F330A and W333I amino acid substitutions relative to the sequence of SEQ ID NO: 1. In some embodiments, the PGA comprises M182I and F330A amino acid substitutions relative to the sequence of SEQ ID NO: 1. In some embodiments, the PGA comprises a sequence that is at least 70%, at least 75%, at least 80%, at least 85%, at least 90%, or at least 95% identical to SEQ ID NO: 1 or SEQ ID NO: 83.

In some embodiments, the PGA comprises an alpha subunit and a beta subunit, wherein the sequence of the alpha subunit is at least 70%, at least 75%, at least 80%, at least 85%, at least 90%, or at least 95% identical the sequence of any one of SEQ ID NOs: 3-6, 8, 10, 13, 18, 20, 21, 121, 123, 125, 127, 129, 131, or 133. In some embodiments, the PGA comprises an alpha subunit and a beta subunit, wherein the sequence of the alpha subunit is at least 70%, at least 75%, at least 80%, at least 85%, at least 90%, or at least 95% identical to the sequence of any one of SEQ ID NOs: 3-6, 8, 10, 13, 18, 20, 21, 121, 123, 125, 127, 129, 131, or 133. In some embodiments, the sequence of the beta subunit is at least 70%, at least 75%, at least 80%, at least 85%, at least 90%, or at least 95% identical to the sequence of any one of SEQ ID NOs: 23-25, 27-32, 34-39, or 88. In some embodiments, the sequence of the beta subunit is at least 70%, at least 75%, at least 80%, at least 85%, at least 90%, or at least 95% identical to the sequence of any one of SEQ ID NOs: 23-25, 27-32, 34-39, or 88.

In some embodiments, the PGA further comprises a signal peptide. In some embodiments, the PGA further comprises a spacer interposed between the alpha subunit and the beta subunit. In some embodiments, the signal peptide is the signal peptide of a PGA from bacterial strain Achromobacter sp. CCM4824. In some embodiments, the signal peptide is not the signal peptide of a PGA from bacterial strain Achromobacter sp. CCM4824. In some embodiments, the signal peptide is the signal peptide of a PGA from an Achromobacter bacterial strain that is not Achromobacter sp. CCM4824. In some embodiments, the signal peptide is the signal peptide of a PGA from a source organism other than an Achromobacter bacterial strain. In some embodiments, the signal peptide is not derived from a PGA enzyme.

In some embodiments, the PGA comprises an alpha subunit and a beta subunit, wherein the sequence of the alpha subunit is or comprises SEQ ID NO: 3 and the sequence of the beta subunit is or comprises SEQ ID NO: 23. In some embodiments, the PGA comprises an alpha subunit and a beta subunit, wherein the sequence of the alpha subunit is or comprises SEQ ID NO: 4 and the sequence of the beta subunit is or comprises SEQ ID NO: 24. In some embodiments, the PGA comprises an alpha subunit and a beta subunit, wherein the sequence of the alpha subunit is or comprises SEQ ID NO: 5 and the sequence of the beta subunit is or comprises SEQ ID NO: 25. In some embodiments, the PGA comprises an alpha subunit and a beta subunit, wherein the sequence of the alpha subunit is or comprises SEQ ID NO: 6 and the sequence of the beta subunit is or comprises SEQ ID NO: 23. In some embodiments, the PGA comprises an alpha subunit and a beta subunit, wherein the sequence of the alpha subunit is or comprises SEQ ID NO: 5 and the sequence of the beta subunit is or comprises SEQ ID NO: 27. In some embodiments, the PGA comprises an alpha subunit and a beta subunit, wherein the sequence of the alpha subunit is or comprises SEQ ID NO: 8 and the sequence of the beta subunit is or comprises SEQ ID NO: 28. In some embodiments, the PGA comprises an alpha subunit and a beta subunit, wherein the sequence of the alpha subunit is or comprises SEQ ID NO: 5 and the sequence of the beta subunit is or comprises SEQ ID NO: 29. In some embodiments, the PGA comprises an alpha subunit and a beta subunit, wherein the sequence of the alpha subunit is or comprises SEQ ID NO: 10 and the sequence of the beta subunit is or comprises SEQ ID NO: 30. In some embodiments, the PGA comprises an alpha subunit and a beta subunit, wherein the sequence of the alpha subunit is or comprises SEQ ID NO: 4 and the sequence of the beta subunit is or comprises SEQ ID NO: 31. In some embodiments, the PGA comprises an alpha subunit and a beta subunit, wherein the sequence of the alpha subunit is or comprises SEQ ID NO: 5 and the sequence of the beta subunit is or comprises SEQ ID NO: 32. In some embodiments, the PGA comprises an alpha subunit and a beta subunit, wherein the sequence of the alpha subunit is or comprises SEQ ID NO: 13 and the sequence of the beta subunit is or comprises SEQ ID NO: 23. In some embodiments, the PGA comprises an alpha subunit and a beta subunit, wherein the sequence of the alpha subunit is or comprises SEQ ID NO: 5 and the sequence of the beta subunit is or comprises ID NO: 34. In some embodiments, the PGA comprises an alpha subunit and a beta subunit, wherein the sequence of the alpha subunit is or comprises SEQ ID NO: 4 and the sequence of the beta subunit is or comprises SEQ ID NO: 35. In some embodiments, the PGA comprises an alpha subunit and a beta subunit, wherein the sequence of the alpha subunit is or comprises SEQ ID NO: 4 and the sequence of the beta subunit is or comprises SEQ ID NO: 36. In some embodiments, the PGA comprises an alpha subunit and a beta subunit, wherein the sequence of the alpha subunit is or comprises SEQ ID NO: 5 and the sequence of the beta subunit is or comprises ID NO: 37. In some embodiments, the PGA comprises an alpha subunit and a beta subunit, wherein the sequence of the alpha subunit is or comprises SEQ ID NO: 18 and the sequence of the beta subunit is or comprises SEQ ID NO: 38. In some embodiments, the PGA comprises an alpha subunit and a beta subunit, wherein the sequence of the alpha subunit is or comprises SEQ ID NO: 5 and the sequence of the beta subunit is or comprises SEQ ID NO: 39. In some embodiments, the PGA comprises an alpha subunit and a beta subunit, wherein the sequence of the alpha subunit is or comprises SEQ ID NO: 20 and the sequence of the beta subunit is or comprises SEQ ID NO: 28. In some embodiments, the PGA comprises an alpha subunit and a beta subunit, wherein the sequence of the alpha subunit is or comprises SEQ ID NO: 21 and the sequence of the beta subunit is or comprises SEQ ID NO: 30. In some embodiments, the PGA comprises an alpha subunit and a beta subunit, wherein the sequence of the alpha subunit is or comprises SEQ ID NO: 18 and the sequence of the beta subunit is or comprises SEQ ID NO: 30.

In some embodiments, the spacer sequence comprises a sequence that is at least 70%, at least 75%, at least 80%, at least 85%, at least 90%, or at least 95% identical to any one of SEQ ID NOs: 87, 95, 97, or 99. In some embodiments, the spacer sequence comprises a sequence that is at least 70%, at least 75%, at least 80%, at least 85%, at least 90%, or at least 95% identical to any one of SEQ ID NOs: 87, 95, 97, or 99.

In some embodiments, the PGA comprises a beta subunit, wherein the beta subunit comprises an amino acid substitution at a residue corresponding to amino acid position 330 in SEQ ID NO: 1. In some embodiments, the PGA comprises a beta subunit, wherein the beta subunit comprises an alanine (A) at the residue corresponding to amino acid position 330 in SEQ ID NO: 1.

Aspects of the present disclosure relate to a penicillin G acylase (PGA), comprising: an alpha subunit and a beta subunit; wherein the alpha subunit comprises an amino acid substitution at one or more of the following amino acid residues relative to the sequence of SEQ ID NO: 1: T90, T181, and/or A189; and/or comprises one of the following amino acid substitutions relative to the sequence of SEQ ID NO: 1: M182F, M182I, M182N, or M182T; and/or comprises the following amino acid substitution relative to the sequence of SEQ ID NO: 1: R185F.

In some embodiments, the alpha subunit comprises one or more of the following amino acid substitutions relative to the sequence of SEQ ID NO: 1: T90V, T181M, M182N, M182T, M182I, M182F, R185F, and/or A189V. In some embodiments, the beta subunit comprises an amino acid substitution at one or more of the following amino acid residues relative to the sequence of SEQ ID NO: 1: N326, F330, W333, L362, D380, T482, H498, V563 and/or L770. In some embodiments, the beta subunit comprises one or more of the following amino acid substitutions relative to the sequence of SEQ ID NO: 1: N326A, F330A, F330G, W333Y, W333I, W333P, W333T, L362F, D380Q, D380I, D380V, T482P, H498S, H498L, V563Q, L770T and/or L770A.

Aspects of the present disclosure relate to a penicillin G acylase (PGA), comprising: an alpha subunit and a beta subunit; wherein the beta subunit comprises an amino acid substitution at one or more of the following amino acid residues relative to the sequence of SEQ ID NO: 1: N326, W333, L362, D380, H498, V563; and/or comprises the following amino acid substitution relative to the sequence of SEQ ID NO: 1: T482P; and/or comprises one of the following amino acid substitutions relative to the sequence of SEQ ID NO: 1: L770A or L770T.

In some embodiments, the beta subunit comprises one or more of the following amino acid substitutions relative to the sequence of SEQ ID NO: 1: N326A, F330A, F330G, W333Y, W333I, W333P, W333T, L362F, D380Q, D380I, D380V, T482P, H498S, H498L, V563Q, L770T and/or L770A. In some embodiments, the alpha subunit comprises an amino acid substitution at one or more of the following amino acid residues relative to the sequence of SEQ ID NO: 1: T90, T181, M182, M182, M182, M182, M182, R185, and/or A189. In some embodiments, the alpha subunit comprises one or more of the following amino acid substitutions relative to the sequence of SEQ ID NO: 1: T90V, T181M, M182N, M182L, M182T, M182I, M182F, R185F, and/or A189V. In some embodiments, the alpha subunit comprises a sequence that is at least 70%, at least 75%, at least 80%, at least 85%, at least 90%, or at least 95% identical to any one of SEQ ID NOs: 3-6, 8, 10, 13, 18, 20, 21, 121, 123, 125, 127, 129, 131, or 133. In some embodiments, the beta subunit comprises a sequence that is at least 70%, at least 75%, at least 80%, at least 85%, at least 90%, or at least 95% identical to any one of SEQ ID NOs: 23-25, 27-32, 34-39, or 88.

In some embodiments, the PGA further comprises a signal peptide. In some embodiments, the signal peptide is at the N-terminus of the alpha subunit. In some embodiments, the PGA further comprises a spacer interposed between the alpha subunit and the beta subunit.

In some embodiments, the alpha subunit of the PGA comprises a sequence with at least about 70%, 75%, 80%, 85%, 90%, 91%, 92%, 93%, 94%, 95%, 96%, 97%, 98%, or 99% identity to the sequence of amino acids 22 to 268, 22 to 252, 22 to 249, 22 to 215, 41 to 268, 41 to 252, 41 to 249, or 41 to 215 of SEQ ID NO: 1. In some embodiments, the alpha subunit of the PGA comprises a sequence with at least about 70% identity to the sequence of amino acids 22 to 268, 22 to 252, 22 to 249, 22 to 215, 41 to 268, 41 to 252, 41 to 249, or 41 to 215 of SEQ ID NO: 1. In some embodiments, the alpha subunit of the PGA comprises a sequence with at least about 80% identity to the sequence of amino acids 22 to 268, 22 to 252, 22 to 249, 22 to 215, 41 to 268, 41 to 252, 41 to 249, or 41 to 215 of SEQ ID NO: 1. In some embodiments, the alpha subunit of the PGA comprises a sequence with at least about 90% identity to the sequence of amino acids 22 to 268, 22 to 252, 22 to 249, 22 to 215, 41 to 268, 41 to 252, 41 to 249, or 41 to 215 of SEQ ID NO: 1. In some embodiments, the alpha subunit of the PGA comprises a sequence with at least about 70% identity to the sequence of SEQ ID NO: 4. In some embodiments, the alpha subunit of the PGA comprises a sequence with at least about 70% identity to the sequence of any one of SEQ ID NOs: 18, 23, 44, 51, 66, and/or 77. In some embodiments, the alpha subunit of the PGA comprises a sequence with at least about 80% identity to the sequence of any one of SEQ ID NOs: 18, 23, 44, 51, 66, and/or 77. In some embodiments, the alpha subunit of the PGA comprises a sequence with at least about 90% identity to the sequence of any one of SEQ ID NOs: 18, 23, 44, 51, 66, and/or 77. In some embodiments, the alpha subunit of the PGA comprises a sequence with at least about 95% identity to the sequence of any one of SEQ ID NOs: 18, 23, 44, 51, 66, and/or 77.

In some embodiments, the beta subunit of the PGA comprises a sequence with at least about 80%, 85%, 90%, 91%, 92%, 93%, 94%, 95%, 96%, 97%, 98%, or 99% identity to the sequence of amino acids 307 to 863 of SEQ ID NO: 1.

Aspects of the present disclosure relate to a host cell that comprises a PGA described in this disclosure.

Aspects of the present disclosure relate to a host cell that comprises one or more polynucleotides encoding a PGA described in this disclosure.

In some embodiments, the PGA comprises an alpha subunit and a beta subunit, and wherein the host cell comprises one or more polynucleotides encoding the alpha subunit and the beta subunit of the PGA.

In some embodiments, the host cell is a bacterial cell, an archaebacterial cell, a fungal cell, a yeast cell, an animal cell, a mammalian cell, or a human cell. In some embodiments, the host cell is a bacterial cell. In some embodiments, the bacterial cell is an Escherichia coli (E. coli) cell. In some embodiments, the bacterial cell is a Bacillus cell. In some embodiments, the host cell is a fungal cell. In some embodiments, the host cell is a yeast cell.

In some embodiments, the PGA is able to convert 6-aminopenicillanic acid (6-APA) and D-4-hydroxyphenylglucine methyl ester (D-HPGM) to D-amoxicillin. In some embodiments, the PGA is able to convert 7-aminodeacetoxycephalosporanic acid (7-ADCA) and D-phenylglycine methyl ester (D-PGM) to cephalexin.

Aspects of the present disclosure relate to a method of converting 6-aminopenicillanic acid (6-APA) and D-4-hydroxyphenylglucine methyl ester (D-HPGM) to D-amoxicillin, comprising contacting 6-APA and D-HPGM with a PGA, wherein the PGA comprises the following amino acid substitutions relative to the sequence of SEQ ID NO: 1: (i) F330A and W333I; (ii) M182I and F330A; (iii) T90V, A189V, F330A, W333I and D380V; (iv) T90V, A189V, F330A, D380V, H498L and V563Q; (v) M182L, F330A and T482P; (vi) T90V, A189V, F330A, W333I, D380I and H498L; (vii) M182T, F330A and L362F; (viii) M182L, R185F, F330A and T482P; (ix) F330G; (x) F330A and W333Y; (xi) R185F, N326A, F330A and T482P; (xii) F330A and W333P; (xiii) F330A and L770T; (xiv) T181M and F330A; (xv) F330A and D380Q; (xvi) M182N and F330A; (xvii) R185F, F330A and T482P; (xviii) F330A and L770A; (xix) T90V, A189V, F330A, W333T, D380V and H498S; or (xx) T181M, M182F, F330A and L362F.

Aspects of the present disclosure relate to a method of converting 6-aminopenicillanic acid (6-APA) and D-4-hydroxyphenylglucine methyl ester (D-HPGM) to D-amoxicillin, comprising contacting 6-APA and D-HPGM with a PGA, wherein the PGA comprises an F330A amino acid substitution and a W333I amino acid substitution relative to sequence of SEQ ID NO: 1.

In some embodiments, the PGA comprises an F330A amino acid substitution and a L770A amino acid substitution relative to sequence of SEQ ID NO: 1. In some embodiments, the PGA comprises an F330A amino acid substitution and a L770T amino acid substitution relative to sequence of SEQ ID NO: 1.

Aspects of the present disclosure relate to a method of converting 7-aminodeacetoxycephalosporanic acid (7-ADCA) and D-phenylglycine methyl ester (D-PGM) to cephalexin, comprising contacting 7-ADCA and D-PGM with a PGA, wherein the PGA comprises the following amino acid substitutions relative to the sequence of SEQ ID NO: 1: (i) F330A and W333I; (ii) M182I and F330A; (iii) T90V, A189V, F330A, W333I and D380V; (iv) T90V, A189V, F330A, D380V, H498L and V563Q; (v) M182L, F330A and T482P; (vi) T90V, A189V, F330A, W333I, D380I and H498L; (vii) M182T, F330A and L362F; (viii) M182L, R185F, F330A and T482P; (ix) F330G; (x) F330A and W333Y; (xi) R185F, N326A, F330A and T482P; (xii) F330A and W333P; (xiii) F330A and L770T; (xiv) T181M and F330A; (xv) F330A and D380Q; (xvi) M182N and F330A; (xvii) R185F, F330A and T482P; (xviii) F330A and L770A; (xix) T90V, A189V, F330A, W333T, D380V and H498S; or (xx) T181M, M182F, F330A and L362F.

Aspects of the present disclosure relate to a method of converting 7-aminodeacetoxycephalosporanic acid (7-ADCA) and D-phenylglycine methyl ester (D-PGM) to cephalexin, comprising contacting 7-ADCA and D-PGM with a PGA, wherein the PGA comprises an M182I amino acid substitution and an F330A amino acid substitution relative to sequence of SEQ ID NO: 1.

In some embodiments, the PGA comprises a sequence that is at least 70%, at least 75%, at least 80%, at least 85%, at least 90%, or at least 95% identical to SEQ ID NO: 1 or SEQ ID NO: 83. In some embodiments, the PGA is encoded by a sequence that is at least 70%, at least 75%, at least 80%, at least 85%, at least 90%, or at least 95% identical to SEQ ID NO: 2 or SEQ ID NO: 84.

Aspects of the present disclosure relate to a method of converting a β-lactam precursor to a β-lactam, comprising contacting the β-lactam precursor with a PGA described in this disclosure. In some embodiments, the β-lactam is D-amoxicillin. In some embodiments, the β-lactam is cephalexin. In some embodiments, the β-lactam precursor is one or more of 6-aminopenicillanic acid (6-APA), D-4-hydroxyphenylglucine methyl ester (D-HPGM), 7-aminodeacetoxycephalosporanic acid (7-ADCA), and/or D-phenylglycine methyl ester (D-PGM).

Aspects of the present disclosure relate to a penicillin G acylase (PGA), wherein the PGA comprises the following amino acid substitutions relative to the sequence of SEQ ID NO: 1: (a) F330A; and (b) any one or more of: (i) W333I; (ii) M182I; (iii) T90V, A189V, W333I and D380V; (iv) T90V, A189V, D380V, H498L and V563Q; (v) M182L, T482P; (vi) T90V, A189V, W333I, D380I and H498L; (vii) M182T, L362F; (viii) M182L, R185F, T482P; (ix) W333Y; (x) R185F, N326A and T482P; (xi) W333P; (xii) L770T; (xiii) T181M; (xiv) D380Q; (xv) M182N; (xvi) R185F and T482P; (xvii) L770A; (xviii) T90V, A189V, W333T, D380V and H498S; or (xix) T181M, M182F and L362F.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented in this disclosure. The accompanying drawings are not intended to be drawn to scale. The drawings are illustrative only. For purposes of clarity, not every component may be labeled in every drawing. In the drawings:

FIG. 1 provides a schematic showing a biosynthetic pathway for the production of D-amoxicillin.

FIG. 2 provides a schematic showing a biosynthetic pathway for the production of cephalexin.

FIG. 3 provides a schematic diagram of an expression vector for expressing polynucleotides (indicated as library gene) encoding penicillin G acylase (PGA) variants.

FIG. 4 shows a graph of amoxicillin secondary screening data under experimental Condition 1. D-amoxicillin and D-HPG concentrations at time points 1, 2, and 3 for each enzyme are shown.

FIG. 5 shows a graph of amoxicillin secondary screening data under experimental Condition 2. D-amoxicillin and D-HPG concentrations at time points 1, 2, and 3 for each enzyme are shown.

FIG. 6 shows a graph of cephalexin screening data. Strain ID is plotted against the median normalized ratio of cephalexin formation rate to cephalexin hydrolysis rate.

### DETAILED DESCRIPTION OF THE INVENTION

The present disclosure provides, in some aspects, engineered enzymes that are capable of enhanced production of β-lactam antibiotics, such as D-amoxicillin and cephalexin. These enzymes include penicillin G acylases (PGAs), which are enzymes that catalyze a reaction converting 6-aminopenicillanic acid (6-APA) and D-4-hydroxyphenylglycine methyl ester (D-HPGM) to D-amoxicillin and/or 7-aminodeacetoxycephalosporanic acid (7-ADCA) and D-phenylglycine methyl ester (D-PGM) to cephalexin. The disclosed enzymes and host cells comprising such enzymes may be used to support the medical industry by providing inexpensive β-lactam antibiotics such as D-amoxicillin and/or cephalexin. The disclosure is directed, in part, to the discovery of PGA enzymes capable of converting 6-APA and D-HPGM to D-amoxicillin and/or 7-ADCA and D-PGM to cephalexin, nucleic acids encoding the same, and host cells capable of expressing PGA enzymes.

### β-Lactams

As used in this disclosure, a “β-lactam” refers to a chemical compound that comprises a β-lactam ring in its chemical structure. The structure of a β-lactam ring is provided by Formula (I):

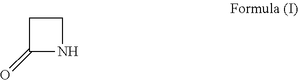

β-lactams are often associated with antibiotics, such as penicillin and penicillin derivatives, cephalosporin and cephalosporin derivatives, and many other widely used antibiotics. As used in this disclosure, a “β-lactam antibiotic” or a “beta-lactam antibiotic” which are used interchangeably, refer to an antibiotic that includes β-lactam ring in its chemical structure. Typical β-lactam antibiotics function by inhibiting the synthesis of the peptidoglycan layer of bacterial cell walls. In some embodiments, the β-lactam antibiotics described in the present disclosure inhibit cell wall synthesis of Gram-positive and/or Gram-negative bacteria. In some embodiments, the β-lactam antibiotics described in the present disclosure inhibit cell wall synthesis of Gram-positive bacteria. In some embodiments, the β-lactam antibiotics described in the present disclosure inhibit cell wall synthesis of Gram-negative bacteria. In some embodiments, the β-lactam antibiotics described in the present disclosure inhibit cell wall synthesis of both Gram-positive and Gram-negative bacteria. In some embodiments, the β-lactam antibiotic is penicillin or a penicillin derivative. Examples of penicillins and penicillin derivatives include but are not limited to: penicillin G, penicillin V, amoxicillin, ampicillin and pivampicillin. In some embodiments, the β-lactam antibiotic is cephalosporin or a cephalosporin derivative. Examples of cephalosporins and cephalosporin derivatives include but are not limited to cephalexin, cefprozil, cefaclor, cephradine, cefadroxil, cefamandole, cephalosporin, cefazolin, cefonicid, cephalothin and cephaloglycin.

The present disclosure is related, at least in part, to production of two β-lactam antibiotics: D-amoxicillin and cephalexin. D-amoxicillin is a widely prescribed antibiotic used in the treatment of a variety of bacterial infections. The chemical structure of D-amoxicillin is provided by Formula (II):

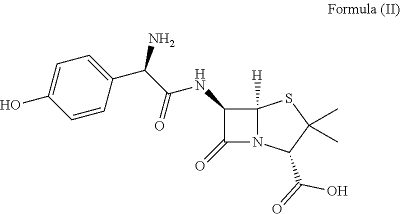 [SMILES: CC1(C)S[C@@H]2[C@H](NC(=O)[C@H](N)c3ccc(O)cc3)C(=O)N2[C@H]1C(=O)O]

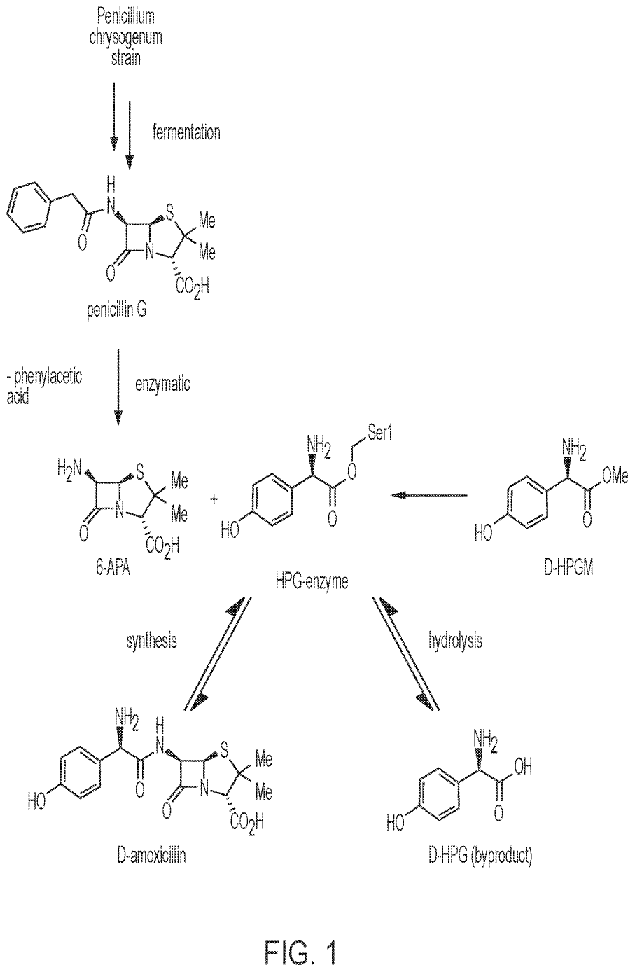

D-amoxicillin is produced commercially through a two-step, semi-synthetic process involving fermentation and in vitro enzymatic synthesis (FIG. 1). In the fermentation step, a Penicillium chrysogenum strain first gencrates penicillin G (PenG) biosynthetically. Following PenG isolation, it is converted enzymatically to 6-aminopenicillanic acid (6-APA) and phenylacetic acid (PAA). 6-APA and D-4-hydroxyphenylglycine methyl ester (D-HPGM) are then subsequently transformed enzymatically to D-amoxicillin by a penicillin G acylase (PGA) enzyme. During this final transformation, the PGA can synthesize the desired product D-amoxicillin or it can hydrolyze the D-HPGM to produce the undesired by-product D-4-hydroxyphenylglycine (D-HPG). Additional methods of producing penicillin and related compounds are known in the art; see, for example: Sawant et al. 2022 Biotech. Letter. 44:179-192; Hassan 2016 Int. J. Cur. Res. Rev. 8:11-22; Avinash et al. 2017 Prep. Bioch. Biotech. 47:52-57; Bruggink et al. 2001 Synthesis of Beta-lactam Antibiotics, p. 13; Koreishi et al. 2007 Biosci. Biotech. Biochem. 71:1582-1586.

Cephalexin is another widely prescribed antibiotic used in the treatment of a variety of bacterial infections. The chemical structure of cephalexin is provided by Formula (III):

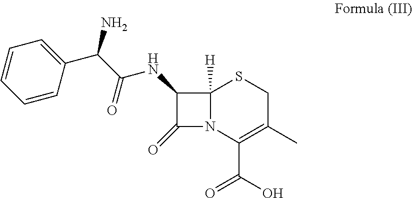 [SMILES: CC1=C(C(=O)O)N2C(=O)[C@@H](NC(=O)[C@H](N)c3ccccc3)[C@H]2SC1]

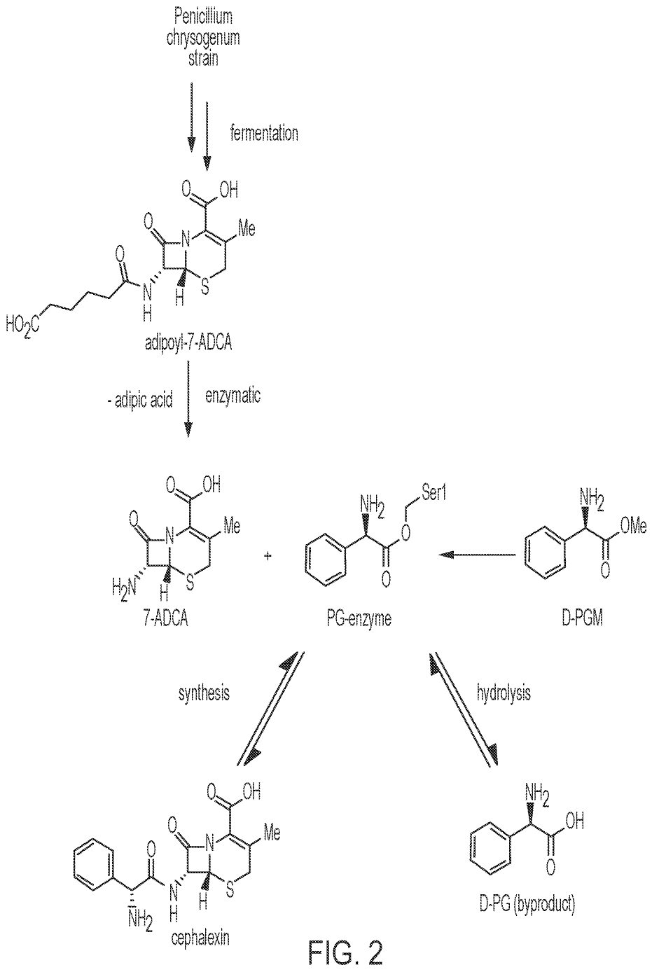

Cephalexin is produced commercially through a two-step, semi-synthetic process involving fermentation and in vitro enzymatic synthesis (FIG. 2). In the fermentation step, a Penicillium chrysogenum strain first generates adipoyl-7-aminodeacetoxycephalosporanic acid (adipoyl-7-ADCA) biosynthetically. Following adipoyl-7-ADCA isolation, it is converted enzymatically to 7-aminodeacetoxycephalosporanic acid (7-ADCA) and adipic acid. 7-ADCA and D-phenylglycine methyl ester (D-PGM) are then subsequently transformed enzymatically to cephalexin by a penicillin G acylase (PGA) enzyme. During this final transformation, the PGA enzyme can synthesize the desired product cephalexin or it can hydrolyze the D-PGM to produce the undesired by-product D-phenylglycine (D-PG). Additional methods of producing cephalexin, cephalosporins and related compounds, including using Acremonium and enzymes derived therefrom, are described in the art; see, for example: Lin et al. 2022 J. Fungi. 8:450; Zhang et al. 2022 Crystals 12:1662; Lin et al. 2015 PNAS 112:9855; Fan et al. 2017 J. Ind. Microb. Biotech. 44:705; Hu et al. 2016 Synth. Syst. Biotech. 1:143; and Hardianto et al. 2016 J. Pure App. Microb. 10:2495.

### Penicillin G Acylase (PGA)

As used in this disclosure, a “penicillin G acylase,” or a “penicillin G acylase enzyme” or a “PGA,” which are used interchangeably, refer to an enzyme that can convert a β-lactam precursor into one or more β-lactams. In some embodiments, a PGA can catalyze the conversion of 6-APA and D-HPGM to D-amoxicillin and/or 7-ADCA and D-PGM to cephalexin. In some embodiments, a PGA converts 6-APA and D-HPGM to D-amoxicillin. In some embodiments, a PGA converts 7-ADCA and D-PGM to cephalexin. In some embodiments, a PGA converts a β-lactam precursor into one or more β-lactams. β-lactams can include but are not limited to: amoxicillin, ampicillin, pivampicillin, cephalexin, cefprozil, cefaclor, cephradine, cefadroxil, cefamandole, cephalosporin, cefazolin, cefonicid, cephalothin, or cephaloglycin. Naturally-occurring PGAs found in bacteria belong to a superfamily of N-terminal nucleophile hydrolases (Grigorenko et al. ACS Catal. 2014, 4, 8, 2521-2529). Such enzymes catalyze selective hydrolysis of the side chain amide bond of penicillins and cephalosporins while leaving the labile amide bond in the β-lactam ring intact.

In some embodiments, a PGA can use 6-APA and/or D-HPGM as substrates. In some embodiments, a PGA exhibits specificity for 6-APA and/or D-HPGM compared to other available substrates. In some embodiments, a PGA can produce D-amoxicillin and/or dihydroxyphenylglycine (D-HPG) from 6-APA and D-HPGM. In some embodiments, D-amoxicillin is produced by a synthesis reaction from 6-APA and D-HPGM. In some embodiments, D-HPG is produced by a hydrolysis reaction from 6-APA and D-HPGM. In some embodiments, D-HPG is an undesirable by-product. In some embodiments, another undesirable but possible side reaction is the hydrolysis of D-amoxicillin to 6-APA and D-HPG. In some embodiments, a PGA predominantly consumes 6-APA and/or D-HPGM relative to one or more other available substrates; e.g., a PGA may consume 6-APA and/or D-HPGM at a rate of at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold higher (e.g., 2-fold to 6-fold more) relative to one or more other available substrates.

In some embodiments, a PGA can use 7-ADCA and/or D-PGM as substrates. In some embodiments, a PGA exhibits specificity for 7-ADCA and/or D-PGM compared to other available substrates. In some embodiments, a PGA can produce cephalexin and/or D-phenylglycine (D-PG) from 7-ADCA and D-PGM. In some embodiments, cephalexin is produced by a synthesis reaction from 7-ADCA and D-PGM. In some embodiments, D-PG is produced by a hydrolysis reaction from 7-ADCA and D-PGM. In some embodiments, D-PG is an undesirable by-product. In some embodiments, another undesirable but possible side reaction is the hydrolysis of cephalexin to 7-ADCA and D-PG. In some embodiments, a PGA predominantly consumes 7-ADCA and/or D-PGM relative to one or more other available substrates; e.g., a PGA may consume 7-ADCA and/or D-PGM at a rate of at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold higher (e.g., 2-fold to 6-fold more) relative to one or more other available substrates.

In some embodiments, a PGA comprises an alpha subunit and a beta subunit. In some embodiments, a PGA comprises, in order from N- to C-terminus: an alpha subunit, a spacer, and a beta subunit. In some embodiments, a PGA comprises, in order from N- to C-terminus: a signal peptide, an alpha subunit, a spacer, and a beta subunit. In some embodiments, a PGA comprises, in order from N- to C-terminus: an optional signal peptide, an alpha subunit, an optional spacer, and a beta subunit, wherein the signal peptide and/or the spacer (if present) can be cleaved, leaving a PGA comprising an alpha and a beta subunit.

In some embodiments, a single coding segment encodes the signal peptide, the alpha subunit, the spacer, and the beta subunit, and after translation, the signal peptide and/or spacer are cleaved, leaving a PGA comprising an alpha subunit and a beta subunit. In some embodiments, the alpha subunit and the beta subunit are encoded by separate coding segments (e.g., on the same or separate nucleic acids), and after protein production, the alpha subunit and beta subunit together form a PGA.

In some embodiments, a PGA is derived from an Achromobacter sp.

In some embodiments, the wild-type PGA is an Achromobacter sp. CCM 4824 PGA provided by SEQ ID NO: 1 below [GenBank: AAY25991.1], which comprises, in order from N- to C-terminus, a signal peptide, an alpha subunit, a spacer, and a beta subunit:

(SEQ ID NO: 1)
MKQQWLSAALLAASSCLPAMAAQPVAPAAGQTSEAVAARPQTADGKVTIRRDAYGMPHVYADTVYGIFYG
YGYAVAQDRLFQMEMARRSTQGRVAEVLGASMVGFDKSIRANFSPERIQRQLAALPAADRQVLDGYAAGM
NAWLARVRAQPGQLMPKEFNDLGFAPADWTAYDVAMIFVGTMANRFSDANSEIDNLALLTALKDRHGAAD
AMRIFNQLRWLTDSRAPTTVPAEAGSYQPPVFQPDGADPLAYALPRYDGTPPMLERVVRDPATRGVVDGA
PATLRAQLAAQYAQSGQPGIAGFPTTSNMWIVGRDHAKDARSILLNGPQFGWWNPAYTYGIGLHGAGFDV
VGNTPFAYPSILFGHNAHVTWGSTAGFGDDVDIFAEKLDPADRTRYFHDGQWKTLEKRTDLILVKDAAPV
TLDVYRSVHGLIVKFDDAQHVAYAKARAWEGYELQSLMAWIRKTQSANWEQWKAQAARHALTINWYYADD
RGNIGYAHTGFYPRRRPGHDPRLPVPGTGEMDWLGLLPFSTNPQVYNPRQGFIANWNNQPMRGYPSTDLF
AIVWGQADRYAEIETRLKAMTANGGKVSAQQMWDLIRTTSYADVNRRHELPFLQRAVQGLPADDPRVRLV
AGLAAWDGMMTSERQPGYFDNAGPAVMDAWLRAMLRRTLADEMPADFFKWYSATGYPTPQAPATGSLNLT
TGVKVLFNALAGPEAGVPQRYDFFNGARADDVILAALDDALAALRQAYGQDPAAWKIPAPPMVFAPKNEL
GVPQADAKAVLCYRATQNRGTENNMTVFDGKSVRAVDVVAPGQSGFVAPDGTPSPHTRDQFDLYNTFGSK
RVWFTADEVRRNATSEETLRYPR

A non-limiting example of a nucleotide sequence encoding SEQ ID NO: 1 is provided by SEQ ID NO: 2:

(SEQ ID NO: 2)
ATGAAGCAGCAATGGTTGTCGGCCGCCCTGTTGGCGGCCAGTTCGTGCCTGCCCGCGATGGCGGCGCAGC
CGGTGGCGCCAGCCGCCGGCCAGACGTCCGAGGCGGTTGCGGCACGGCCCCAAACCGCCGATGGCAAGGT
CACGATCCGGCGCGATGCCTACGGCATGCCGCATGTCTATGCCGACACGGTGTACGGCATCTTCTACGGC
TACGGCTACGCGGTGGCGCAGGACCGGCTGTTCCAGATGGAGATGGCGCGGCGCAGCACCCAGGGCCGGG
TGGCCGAGGTGCTGGGCGCCTCGATGGTGGGCTTCGACAAGTCGATCCGCGCCAATTTCTCGCCCGAGCG
CATCCAGCGCCAGTTGGCGGCGCTGCCGGCCGCCGACCGCCAGGTGCTGGACGGCTACGCGGCTGGCATG
AACGCCTGGCTGGCGCGGGTGCGGGCCCAGCCGGGCCAACTGATGCCCAAGGAATTCAATGACCTGGGTT
TCGCGCCGGCCGACTGGACCGCCTACGACGTGGCGATGATCTTCGTCGGCACCATGGCCAACCGCTTTTC
GGACGCCAACAGCGAGATCGACAACCTGGCGCTGCTGACGGCGTTGAAGGACCGGCATGGCGCCGCCGAT
GCCATGCGCATCTTCAACCAGTTGCGCTGGCTGACCGACAGCCGCGCGCCGACCACGGTGCCGGCCGAAG
CGGGCAGCTACCAGCCGCCGGTGTTCCAGCCGGACGGCGCGGACCCGCTGGCCTACGCGCTGCCGCGCTA
CGACGGCACGCCGCCGATGCTCGAGCGGGTGGTGCGCGACCCGGCCACGCGGGGCGTGGTCGACGGCGCG
CCGGCGACGCTGCGGGCGCAACTGGCCGCCCAATACGCGCAATCGGGCCAGCCCGGCATCGCCGGCTTTC
CGACCACCAGCAATATGTGGATCGTGGGCCGCGACCACGCCAAGGACGCGCGCTCGATCCTGCTGAACGG
CCCGCAGTTCGGCTGGTGGAATCCGGCCTATACCTACGGCATCGGCTTGCACGGCGCCGGCTTCGACGTG
GTCGGCAACACGCCGTTCGCCTATCCCAGCATTCTGTTCGGCCACAATGCACACGTGACGTGGGGTTCGA
CCGCGGGCTTCGGCGATGACGTCGACATCTTTGCCGAAAAGCTCGATCCCGCCGACCGCACGCGCTATTT
CCACGACGGCCAATGGAAGACGCTGGAAAAGCGCACCGACCTGATCCTGGTGAAGGACGCGGCGCCAGTG
ACGCTGGACGTGTACCGCAGCGTGCATGGCCTGATCGTCAAGTTCGACGACGCGCAGCACGTGGCCTACG
CCAAGGCGCGCGCCTGGGAAGGCTATGAACTGCAATCGCTGATGGCCTGGACCCGCAAGACGCAATCGGC
CAACTGGGAACAGTGGAAGGCGCAGGCGGCGCGCCATGCGCTGACCATCAACTGGTACTACGCCGACGAC
CGCGGCAACATTGGCTACGCGCACACGGGCTTCTATCCCAGGCGCCGTCCGGGCCACGATCCGCGCCTGC
CGGTGCCCGGCACCGGCGAGATGGACTGGCTGGGCCTGCTGCCGTTCTCTACCAATCCGCAGGTCTACAA
CCCGCGCCAGGGCTTCATCGCCAACTGGAACAACCAGCCGATGCGCGGCTACCCGTCCACCGACCTGTTC
GCCATCGTCTGGGGCCAGGCCGACCGCTACGCCGAGATCGAGACGCGCCTGAAGGCCATGACCGCGAACG
GAGGCAAGGTCAGCGCGCAGCAGATGTGGGACCTGATCCGCACCACCAGCTACGCCGACGTCAACCGCCG
TCATTTCCTGCCGTTCCTGCAACGCGCGGTGCAAGGGCTGCCGGCGGATGATCCGCGCGTGCGCCTGGTG
GCCGGCCTGGCGGCCTGGGACGGCATGATGACCAGCGAGCGCCAACCGGGTTACTTCGACAACGCCGGCC
CGGCGGTCATGGACGCGTGGCTGCGCGCCATGCTGCGGCGCACGCTGGCCGACGAGATGCCGGCCGACTT
CTTCAAGTGGTACAGCGCCACCGGCTACCCGACACCGCAGGCGCCGGCCACCGGTTCGCTCAACCTGACC
ACCGGCGTCAAGGTGCTGTTCAACGCCCTGGCCGGGCCCGAGGCTGGCGTGCCGCAGCGCTATGACTTCT
TCAACGGCGCGCGCGCCGACGACGTCATCCTCGCGGCGCTGGACGATGCGCTGGCGGCGCTGCGCCAGGC
CTATGGCCAGGATCCGGCGGCATGGAAGATCCCGGCGCCGCCGATGGTGTTCGCGCCCAAGAACTTCCTG
GGCGTGCCGCAGGCCGACGCCAAGGCGGTGCTGTGCTATCGGGCCACGCAGAACCGCGGCACCGAGAACA
ACATGACGGTGTTCGACGGTAAATCGGTGCGCGCGGTGGATGTGGTGGCGCCGGGGCAGAGCGGCTTCGT
CGCCCCGGACGGCACGCCGTCGCCGCACACCCGCGACCAGTTCGACCTGTACAACACCTTCGGCAGCAAA
CGGGTGTGGTTCACGGCCGATGAGGTGCGGCGCAACGCTACGTCGGAAGAGACGTTGCGCTACCCGCGGT
AA

Without wishing to be bound by any particular theory, different boundaries have been reported between the signal peptide and the alpha subunit (e.g., between the C-terminus of the signal peptide and the N-terminus of the alpha subunit) within SEQ ID NO: 1, and between the alpha subunit and the spacer (e.g., between the C-terminus of the alpha subunit and the N-terminus of the spacer) within SEQ ID NO: 1.

In some embodiments, the N-terminus of the signal peptide corresponds to amino acid 1 of SEQ ID NO: 1, the C-terminus of the signal peptide corresponds to amino acid 21 of SEQ ID NO: 1, and the N-terminus of the alpha subunit corresponds to amino acid 22 of SEQ ID NO: 1. See, for example: WO2021096298A1, IN383213B, and KR101985911B1, which are incorporated by reference in this disclosure in their entireties.

In some embodiments, the N-terminus of the signal peptide corresponds to amino acid 1 of SEQ ID NO: 1, the C-terminus of the signal peptide corresponds to amino acid 40 of SEQ ID NO: 1, and the N-terminus of the alpha subunit corresponds to amino acid 41 of SEQ ID NO: 1. See, for example: WO2021140526A1, CN105483105B, and IN202021000782A, which are incorporated by reference in this disclosure in their entireties.

In some embodiments, the C-terminus of the alpha subunit corresponds to amino acid 268 of SEQ ID NO: 1, and the N-terminus of the spacer corresponds to amino acid 269 of SEQ ID NO: 1. See, for example: WO2021096298A1, which is incorporated by reference in this disclosure in its entirety.

In some embodiments, the C-terminus of the alpha subunit corresponds to amino acid 252 of SEQ ID NO: 1, and the N-terminus of the spacer corresponds to amino acid 253 of SEQ ID NO: 1, by analogy with a PGA homolog from Achromobacter xylosoxidans. See, for example: CN105274082B, which is incorporated by reference in this disclosure in its entirety.

In some embodiments, the C-terminus of the alpha subunit corresponds to amino acid 215 of SEQ ID NO: 1, and the N-terminus of the spacer corresponds to amino acid 216 of SEQ ID NO: 1. See, for example: KR101985911B1 and IN383213B, which are incorporated by reference in this disclosure in their entireties.

In some embodiments, the C-terminus of the alpha subunit corresponds to amino acid 249 of SEQ ID NO: 1, and the N-terminus of the spacer corresponds to amino acid 250 of SEQ ID NO: 1. See, for example: WO2021140526A1 and IN202021000782A, which are incorporated by reference in this disclosure in their entireties.

In some embodiments, the N-terminus of the signal peptide corresponds to amino acid 1 of SEQ ID NO: 1, the C-terminus of the signal peptide corresponds to amino acid 21 of SEQ ID NO: 1, the N-terminus of the alpha subunit corresponds to amino acid 22 of SEQ ID NO: 1, the C-terminus of the alpha subunit corresponds to amino acid 268 of SEQ ID NO: 1, and the N-terminus of the spacer corresponds to amino acid 269 of SEQ ID NO: 1. See, for example: WO2021096298A1, which is incorporated by reference in this disclosure in its entirety.

In some embodiments, the N-terminus of the signal peptide corresponds to amino acid 1 of SEQ ID NO: 1, the C-terminus of the signal peptide corresponds to amino acid 21 of SEQ ID NO: 1, the N-terminus of the alpha subunit corresponds to amino acid 22 of SEQ ID NO: 1, the C-terminus of the alpha subunit corresponds to amino acid 252 of SEQ ID NO: 1, and the N-terminus of the spacer corresponds to amino acid 253 of SEQ ID NO: 1, by analogy with a PGA homolog from Achromobacter xylosoxidans. See, for example: CN105274082B, which is incorporated by reference in this disclosure in its entirety.

In some embodiments, the N-terminus of the signal peptide corresponds to amino acid 1 of SEQ ID NO: 1, the C-terminus of the signal peptide corresponds to amino acid 21 of SEQ ID NO: 1, the N-terminus of the alpha subunit corresponds to amino acid 22 of SEQ ID NO: 1, the C-terminus of the alpha subunit corresponds to amino acid 215 of SEQ ID NO: 1, and the N-terminus of the spacer corresponds to amino acid 216 of SEQ ID NO: 1. See, for example: KR101985911B1 and IN383213B, which are incorporated by reference in this disclosure in their entireties.

In some embodiments, the N-terminus of the signal peptide corresponds to amino acid 1 of SEQ ID NO: 1, the C-terminus of the signal peptide corresponds to amino acid 40 of SEQ ID NO: 1, the N-terminus of the alpha subunit corresponds to amino acid 41 of SEQ ID NO: 1, the C-terminus of the alpha subunit corresponds to amino acid 249 of SEQ ID NO: 1, and the N-terminus of the spacer corresponds to amino acid 250 of SEQ ID NO: 1. See, for example: WO2021140526A1 and IN202021000782A, which are incorporated by reference in this disclosure in their entireties.

In some embodiments, the N-terminus of the signal peptide corresponds to amino acid 1 of SEQ ID NO: 1, the C-terminus of the signal peptide corresponds to amino acid 40 of SEQ ID NO: 1, the N-terminus of the alpha subunit corresponds to amino acid 41 of SEQ ID NO: 1, the C-terminus of the alpha subunit corresponds to amino acid 215 of SEQ ID NO: 1, and the N-terminus of the spacer corresponds to amino acid 216 of SEQ ID NO: 1. See, for example: WO2021140526A1; IN202021000782A; IN383213B; and KR101985911B1, which are incorporated by reference in this disclosure in their entireties.

In some embodiments, the sequence of the signal peptide of a PGA is or comprises the sequence of amino acids 1 to 21 of SEQ ID NO: 1.

In some embodiments, the sequence of the signal peptide of a PGA is or comprises the sequence of amino acids 1 to 40 of SEQ ID NO: 1.

In some embodiments, the sequence of the alpha subunit of a PGA is or comprises the sequence of amino acids 22 to 268 of SEQ ID NO: 1, wherein the alpha subunit optionally comprises any 1 or more of the amino acid substitutions described in this disclosure.

In some embodiments, the sequence of the alpha subunit of a PGA is or comprises the sequence of amino acids 22 to 252 of SEQ ID NO: 1, wherein the alpha subunit optionally comprises any 1 or more of the amino acid substitutions described in this disclosure.

In some embodiments, the sequence of the alpha subunit of a PGA is or comprises the sequence of amino acids 22 to 249 of SEQ ID NO: 1, wherein the alpha subunit optionally comprises any 1 or more of the amino acid substitutions described in this disclosure.

In some embodiments, the sequence of the alpha subunit of a PGA is or comprises the sequence of amino acids 22 to 215 of SEQ ID NO: 1, wherein the alpha subunit optionally comprises any 1 or more of the amino acid substitutions described in this disclosure.

In some embodiments, the sequence of the alpha subunit of a PGA is or comprises the sequence of amino acids 41 to 268 of SEQ ID NO: 1, wherein the alpha subunit optionally comprises any 1 or more of the amino acid substitutions described in this disclosure.

In some embodiments, the sequence of the alpha subunit of a PGA is or comprises the sequence of amino acids 41 to 252 of SEQ ID NO: 1, wherein the alpha subunit optionally comprises any 1 or more of the amino acid substitutions described in this disclosure.

In some embodiments, the sequence of the alpha subunit of a PGA is or comprises the sequence of amino acids 41 to 249 of SEQ ID NO: 1, wherein the alpha subunit optionally comprises any 1 or more of the amino acid substitutions described in this disclosure.

In some embodiments, the sequence of the alpha subunit of a PGA is or comprises the sequence of amino acids 41 to 215 of SEQ ID NO: 1, wherein the alpha subunit optionally comprises any 1 or more of the amino acid substitutions described in this disclosure.

In some embodiments, the sequence of the alpha subunit of a PGA is or comprises the sequence of amino acids 22 to 268 of SEQ ID NO: 1, wherein the alpha subunit comprises any 1 or more of the amino acid substitutions described in this disclosure.

In some embodiments, the sequence of the alpha subunit of a PGA is or comprises the sequence of amino acids 22 to 252 of SEQ ID NO: 1, wherein the alpha subunit comprises any 1 or more of the amino acid substitutions described in this disclosure.

In some embodiments, the sequence of the alpha subunit of a PGA is or comprises the sequence of amino acids 22 to 249 of SEQ ID NO: 1, wherein the alpha subunit comprises any 1 or more of the amino acid substitutions described in this disclosure.

In some embodiments, the sequence of the alpha subunit of a PGA is or comprises the sequence of amino acids 22 to 215 of SEQ ID NO: 1, wherein the alpha subunit comprises any 1 or more of the amino acid substitutions described in this disclosure.

In some embodiments, the sequence of the alpha subunit of a PGA is or comprises the sequence of amino acids 41 to 268 of SEQ ID NO: 1, wherein the alpha subunit comprises any 1 or more of the amino acid substitutions described in this disclosure.

In some embodiments, the sequence of the alpha subunit of a PGA is or comprises the sequence of amino acids 41 to 252 of SEQ ID NO: 1, wherein the alpha subunit comprises any 1 or more of the amino acid substitutions described in this disclosure.

In some embodiments, the sequence of the alpha subunit of a PGA is or comprises the sequence of amino acids 41 to 249 of SEQ ID NO: 1, wherein the alpha subunit comprises any 1 or more of the amino acid substitutions described in this disclosure.

In some embodiments, the sequence of the alpha subunit of a PGA is or comprises the sequence of amino acids 41 to 215 of SEQ ID NO: 1, wherein the alpha subunit comprises any 1 or more of the amino acid substitutions described in this disclosure.

In some embodiments, a PGA comprises an alpha subunit and a beta subunit, wherein the sequence of the alpha subunit is or comprises the sequence of amino acids 22 to 268, amino acids 22 to 252, amino acids 22 to 249, amino acids 22 to 215, amino acids 41 to 268, amino acids 41 to 252, amino acids 41 to 249, or amino acids 41 to 215 of SEQ ID NO: 1, and wherein the alpha and/or beta subunit comprise any 1 or more of the amino acid substitutions described in this disclosure.

In some embodiments, the sequence of a PGA alpha subunit is or comprises a sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, including all values or ranges in between, to a sequence which is or comprises the sequence of amino acids 22 to 268, 22 to 252, 22 to 249, 22 to 215, 41 to 268, 41 to 252, 41 to 249, or 41 to 215 of SEQ ID NO: 1, wherein the alpha subunit optionally comprises any 1 or more of the amino acid substitutions described in this disclosure.

In some embodiments, the sequence of a PGA alpha subunit is or comprises a sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, including all values or ranges in between, to a sequence which is or comprises the sequence of amino acids 22 to 268, 22 to 252, 22 to 249, 22 to 215, 41 to 268, 41 to 252, 41 to 249, or 41 to 215 of SEQ ID NO: 1, wherein the alpha subunit comprises any 1 or more of the amino acid substitutions described in this disclosure.

In some embodiments, the sequence of a PGA alpha subunit is or comprises a sequence having at least about 60%, 65%, 70%, 75%, 80%, 85%, 90%, 91%, 92%, 93%, 94%, 95%, 96%, 97%, 98%, or 99% identity to a sequence which is or comprises the sequence of amino acids 22 to 268, 22 to 252, 22 to 249, 22 to 215, 41 to 268, 41 to 252, 41 to 249, or 41 to 215 of SEQ ID NO: 1, wherein the alpha subunit optionally comprises any 1 or more of the amino acid substitutions described in this disclosure.

In some embodiments, the sequence of a PGA alpha subunit is or comprises a sequence having at least about 60%, 65%, 70%, 75%, 80%, 85%, 90%, 91%, 92%, 93%, 94%, 95%, 96%, 97%, 98%, or 99% identity to a sequence which is or comprises the sequence of amino acids 22 to 268, 22 to 252, 22 to 249, 22 to 215, 41 to 268, 41 to 252, 41 to 249, or 41 to 215 of SEQ ID NO: 1, wherein the alpha subunit comprises any 1 or more of the amino acid substitutions described in this disclosure.

In some embodiments, the sequence of a PGA alpha subunit is or comprises a sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, including all values or ranges in between, to a sequence which is or comprises the sequence of amino acids 22 to 268, 22 to 252, 22 to 249, 22 to 215, 41 to 268, 41 to 252, 41 to 249, or 41 to 215 of SEQ ID NO: 1, wherein the alpha subunit optionally comprises any 2 or more of the amino acid substitutions described in this disclosure.

In some embodiments, the sequence of a PGA alpha subunit is or comprises a sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, including all values or ranges in between, to a sequence which is or comprises the sequence of amino acids 22 to 268, 22 to 252, 22 to 249, 22 to 215, 41 to 268, 41 to 252, 41 to 249, or 41 to 215 of SEQ ID NO: 1, wherein the alpha subunit comprises any 2 or more of the amino acid substitutions described in this disclosure.

In some embodiments, the sequence of a PGA alpha subunit is or comprises a sequence having at least about 60%, 65%, 70%, 75%, 80%, 85%, 90%, 91%, 92%, 93%, 94%, 95%, 96%, 97%, 98%, or 99% identity to a sequence which is or comprises the sequence of amino acids 22 to 268, 22 to 252, 22 to 249, 22 to 215, 41 to 268, 41 to 252, 41 to 249, or 41 to 215 of SEQ ID NO: 1, wherein the alpha subunit optionally comprises any 2 or more of the amino acid substitutions described in this disclosure.

In some embodiments, the sequence of a PGA alpha subunit is or comprises a sequence having at least about 60%, 65%, 70%, 75%, 80%, 85%, 90%, 91%, 92%, 93%, 94%, 95%, 96%, 97%, 98%, or 99% identity to a sequence which is or comprises the sequence of amino acids 22 to 268, 22 to 252, 22 to 249, 22 to 215, 41 to 268, 41 to 252, 41 to 249, or 41 to 215 of SEQ ID NO: 1, wherein the alpha subunit comprises any 2 or more of the amino acid substitutions described in this disclosure.

Without wishing to be bound by any particular theory, the present disclosure notes that there is agreement among the identified literature that the N-terminus of the beta subunit corresponds to amino acid 307 of SEQ ID NO: 1, and the C-terminus of the beta subunit corresponds to amino acid 863 of SEQ ID NO: 1 (e.g., the C-terminal amino acid of the beta subunit is the C-terminal amino acid of SEQ ID NO: 1). See, for example: WO2021096298A1; WO2021140526A1; KR101985911B1; IN202021000782A; IN383213B; CN105274082B; and CN105483105B, which are incorporated by reference in this disclosure in their entireties.

In some embodiments, the sequence of a PGA beta subunit is or comprises a sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, including all values or ranges in between, to a sequence which is or comprises the sequence of amino acids 307 to 863 of SEQ ID NO: 1, wherein the beta subunit optionally comprises any 1 or more of the amino acid substitutions described in this disclosure.

In some embodiments, the sequence of a PGA beta subunit is or comprises a sequence having at least about 60%, 65%, 70%, 75%, 80%, 85%, 90%, 91%, 92%, 93%, 94%, 95%, 96%, 97%, 98%, or 99% identity to a sequence which is or comprises the sequence of amino acids 307 to 863 of SEQ ID NO: 1, wherein the beta subunit optionally comprises any 1 or more of the amino acid substitutions described in this disclosure.

In some embodiments, the sequence of a PGA beta subunit is or comprises a sequence having at least about 60%, 65%, 70%, 75%, 80%, 85%, 90%, 91%, 92%, 93%, 94%, 95%, 96%, 97%, 98%, or 99% identity to a sequence which is or comprises the sequence of amino acids 307 to 863 of SEQ ID NO: 1, wherein the beta subunit comprises any 1 or more of the amino acid substitutions described in this disclosure.

In some embodiments, the sequence of a PGA beta subunit is or comprises a sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, including all values or ranges in between, to a sequence which is or comprises the sequence of amino acids 307 to 863 of SEQ ID NO: 1, wherein the beta subunit comprises an F330A amino acid substitution relative to the sequence of SEQ ID NO: 1 and optionally any 1 or more of the other amino acid substitutions described in this disclosure.

In some embodiments, the sequence of a PGA beta subunit is or comprises a sequence having at least about 60%, 65%, 70%, 75%, 80%, 85%, 90%, 91%, 92%, 93%, 94%, 95%, 96%, 97%, 98%, or 99% identity to a sequence which is or comprises the sequence of amino acids 307 to 863 of SEQ ID NO: 1, wherein the beta subunit comprises an F330A amino acid substitution relative to the sequence of SEQ ID NO: 1 and optionally any 1 or more of the other amino acid substitutions described in this disclosure.

In some embodiments, the sequence of a PGA beta subunit is or comprises a sequence having at least about 60%, 65%, 70%, 75%, 80%, 85%, 90%, 91%, 92%, 93%, 94%, 95%, 96%, 97%, 98%, or 99% identity to a sequence which is or comprises the sequence of amino acids 307 to 863 of SEQ ID NO: 1, wherein the beta subunit comprises an F330A amino acid substitution relative to the sequence of SEQ ID NO: 1 and any 1 or more of the other amino acid substitutions described in this disclosure.

In some embodiments, the sequence of the beta subunit of a PGA is or comprises the sequence of amino acids 307 to 863 of SEQ ID NO: 1, wherein the alpha subunit optionally comprises any 2 or more of the amino acid substitutions described in this disclosure.

In some embodiments, the sequence of the beta subunit of a PGA is or comprises the sequence of amino acids 307 to 863 of SEQ ID NO: 1, wherein the alpha subunit comprises any 2 or more of the amino acid substitutions described in this disclosure.

In some embodiments, the sequence of the signal peptide is or comprises the sequence of amino acids 1 to 21 of SEQ ID NO: 1: MKQQWLSAALLAASSCLPAMA (SEQ ID NO: 118).

In some embodiments, the sequence of the signal peptide is or comprises the sequence of amino acids 1 to 40 of SEQ ID NO: 1:

(SEQ ID NO: 119)
MKQQWLSAALLAASSCLPAMAAQPVAPAAGQTSEAVAARP

In some embodiments, the PGA comprises a sequence corresponding to amino acids 22 to 863 of SEQ ID NO: 1, provided below as SEQ ID NO: 83:

(SEQ ID NO: 83)
AQPVAPAAGQTSEAVAARPQTADGKVTIRRDAYGMPHVYADTVYGIFYGY
GYAVAQDRLFQMEMARRSTQGRVAEVLGASMVGFDKSIRANFSPERIQRQ
LAALPAADRQVLDGYAAGMNAWLARVRAQPGQLMPKEFNDLGFAPADWTA
YDVAMIFVGTMANRFSDANSEIDNLALLTALKDRHGAADAMRIFNQLRWL
TDSRAPTTVPAEAGSYQPPVFQPDGADPLAYALPRYDGTPPMLERVVRDP
ATRGVVDGAPATLRAQLAAQYAQSGQPGIAGFPTTSNMWIVGRDHAKDAR
SILLNGPQFGWWNPAYTYGIGLHGAGFDVVGNTPFAYPSILFGHNAHVTW
GSTAGFGDDVDIFAEKLDPADRTRYFHDGQWKTLEKRTDLILVKDAAPVT
LDVYRSVHGLIVKFDDAQHVAYAKARAWEGYELQSLMAWTRKTQSANWEQ
WKAQAARHALTINWYYADDRGNIGYAHTGFYPRRRPGHDPRLPVPGTGEM
DWLGLLPFSTNPQVYNPRQGFIANWNNQPMRGYPSTDLFAIVWGQADRYA
EIETRLKAMTANGGKVSAQQMWDLIRTTSYADVNRRHFLPFLQRAVQGLP
ADDPRVRLVAGLAAWDGMMTSERQPGYFDNAGPAVMDAWLRAMLRRTLAD
EMPADFFKWYSATGYPTPQAPATGSLNLTTGVKVLFNALAGPEAGVPQRY
DFFNGARADDVILAALDDALAALRQAYGQDPAAWKIPAPPMVFAPKNFLG
VPQADAKAVLCYRATQNRGTENNMTVFDGKSVRAVDVVAPGQSGFVAPDG
TPSPHTRDQFDLYNTFGSKRVWFTADEVRRNATSEETLRYPR

A non-limiting example of a nucleotide sequence encoding SEQ ID NO: 83 is provided by SEQ ID NO: 84:

(SEQ ID NO: 84)
GCGCAGCCGGTGGCGCCAGCCGCCGGCCAGACGTCCGAGGCGGTTGCGGC
ACGGCCCCAAACCGCCGATGGCAAGGTCACGATCCGGCGCGATGCCTACG
GCATGCCGCATGTCTATGCCGACACGGTGTACGGCATCTTCTACGGCTAC
GGCTACGCGGTGGCGCAGGACCGGCTGTTCCAGATGGAGATGGCGCGGCG
CAGCACCCAGGGCCGGGTGGCCGAGGTGCTGGGCGCCTCGATGGTGGGCT
TCGACAAGTCGATCCGCGCCAATTTCTCGCCCGAGCGCATCCAGCGCCAG
TTGGCGGCGCTGCCGGCCGCCGACCGCCAGGTGCTGGACGGCTACGCGGC
TGGCATGAACGCCTGGCTGGCGCGGGTGCGGGCCCAGCCGGGCCAACTGA
TGCCCAAGGAATTCAATGACCTGGGTTTCGCGCCGGCCGACTGGACCGCC
TACGACGTGGCGATGATCTTCGTCGGCACCATGGCCAACCGCTTTTCGGA
CGCCAACAGCGAGATCGACAACCTGGCGCTGCTGACGGCGTTGAAGGACC
GGCATGGCGCCGCCGATGCCATGCGCATCTTCAACCAGTTGCGCTGGCTG
ACCGACAGCCGCGCGCCGACCACGGTGCCGGCCGAAGCGGGCAGCTACCA
GCCGCCGGTGTTCCAGCCGGACGGCGCGGACCCGCTGGCCTACGCGCTGC
CGCGCTACGACGGCACGCCGCCGATGCTCGAGCGGGTGGTGCGCGACCCG
GCCACGCGGGGCGTGGTCGACGGCGCGCCGGCGACGCTGCGGGCGCAACT
GGCCGCCCAATACGCGCAATCGGGCCAGCCCGGCATCGCCGGCTTTCCGA
CCACCAGCAATATGTGGATCGTGGGCCGCGACCACGCCAAGGACGCGCGC
TCGATCCTGCTGAACGGCCCGCAGTTCGGCTGGTGGAATCCGGCCTATAC
CTACGGCATCGGCTTGCACGGCGCCGGCTTCGACGTGGTCGGCAACACGC
CGTTCGCCTATCCCAGCATTCTGTTCGGCCACAATGCACACGTGACGTGG
GGTTCGACCGCGGGCTTCGGCGATGACGTCGACATCTTTGCCGAAAAGCT
CGATCCCGCCGACCGCACGCGCTATTTCCACGACGGCCAATGGAAGACGC
TGGAAAAGCGCACCGACCTGATCCTGGTGAAGGACGCGGCGCCAGTGACG
CTGGACGTGTACCGCAGCGTGCATGGCCTGATCGTCAAGTTCGACGACGC
GCAGCACGTGGCCTACGCCAAGGCGCGCGCCTGGGAAGGCTATGAACTGC
AATCGCTGATGGCCTGGACCCGCAAGACGCAATCGGCCAACTGGGAACAG
TGGAAGGCGCAGGCGGCGCGCCATGCGCTGACCATCAACTGGTACTACGC
CGACGACCGCGGCAACATTGGCTACGCGCACACGGGCTTCTATCCCAGGC
GCCGTCCGGGCCACGATCCGCGCCTGCCGGTGCCCGGCACCGGCGAGATG
GACTGGCTGGGCCTGCTGCCGTTCTCTACCAATCCGCAGGTCTACAACCC
GCGCCAGGGCTTCATCGCCAACTGGAACAACCAGCCGATGCGCGGCTACC
CGTCCACCGACCTGTTCGCCATCGTCTGGGGCCAGGCCGACCGCTACGCC
GAGATCGAGACGCGCCTGAAGGCCATGACCGCGAACGGAGGCAAGGTCAG
CGCGCAGCAGATGTGGGACCTGATCCGCACCACCAGCTACGCCGACGTCA
ACCGCCGTCATTTCCTGCCGTTCCTGCAACGCGCGGTGCAAGGGCTGCCG
GCGGATGATCCGCGCGTGCGCCTGGTGGCCGGCCTGGCGGCCTGGGACGG
CATGATGACCAGCGAGCGCCAACCGGGTTACTTCGACAACGCCGGCCCGG
CGGTCATGGACGCGTGGCTGCGCGCCATGCTGCGGCGCACGCTGGCCGAC
GAGATGCCGGCCGACTTCTTCAAGTGGTACAGCGCCACCGGCTACCCGAC
ACCGCAGGCGCCGGCCACCGGTTCGCTCAACCTGACCACCGGCGTCAAGG
TGCTGTTCAACGCCCTGGCCGGGCCCGAGGCTGGCGTGCCGCAGCGCTAT
GACTTCTTCAACGGCGCGCGCGCCGACGACGTCATCCTCGCGGCGCTGGA
CGATGCGCTGGCGGCGCTGCGCCAGGCCTATGGCCAGGATCCGGCGGCAT
GGAAGATCCCGGCGCCGCCGATGGTGTTCGCGCCCAAGAACTTCCTGGGC
GTGCCGCAGGCCGACGCCAAGGCGGTGCTGTGCTATCGGGCCACGCAGAA
CCGCGGCACCGAGAACAACATGACGGTGTTCGACGGTAAATCGGTGCGCG
CGGTGGATGTGGTGGCGCCGGGGCAGAGCGGCTTCGTCGCCCCGGACGGC
ACGCCGTCGCCGCACACCCGCGACCAGTTCGACCTGTACAACACCTTCGG
CAGCAAACGGGTGTGGTTCACGGCCGATGAGGTGCGGCGCAACGCTACGT
CGGAAGAGACGTTGCGCTACCCGCGGTAA

In some embodiments, the PGA sequence includes an F330A amino acid substitution relative to the sequence of SEQ ID NO: 1 (e.g., the phenylalanine (F) at position 330 of SEQ ID NO: 1 is changed to alanine (A)). Other amino acid substitutions described in this disclosure follow similar nomenclature. In some embodiments, the PGA comprises the sequence of SEQ ID NO: 85 (e.g., amino acids 22 to 863 of SEQ ID NO: 1, comprising in order from N- to C-terminus, an alpha subunit, a spacer, and a beta subunit, and including the amino acid substitution F330A relative to the sequence of SEQ ID NO: 1):

(SEQ ID NO: 85)
AQPVAPAAGQTSEAVAARPQTADGKVTIRRDAYGMPHVYADTVYGIFYGY
GYAVAQDRLFQMEMARRSTQGRVAEVLGASMVGFDKSIRANFSPERIQRQ
LAALPAADRQVLDGYAAGMNAWLARVRAQPGQLMPKEFNDLGFAPADWTA
YDVAMIFVGTMANRFSDANSEIDNLALLTALKDRHGAADAMRIFNQLRWL
TDSRAPTTVPAEAGSYQPPVFQPDGADPLAYALPRYDGTPPMLERVVRDP
ATRGVVDGAPATLRAQLAAQYAQSGQPGIAGFPTTSNMWIVGRDHAKDAR
SILLNGPQAGWWNPAYTYGIGLHGAGFDVVGNTPFAYPSILFGHNAHVTW
GSTAGFGDDVDIFAEKLDPADRTRYFHDGQWKTLEKRTDLILVKDAAPVT
LDVYRSVHGLIVKFDDAQHVAYAKARAWEGYELQSLMAWTRKTQSANWEQ
WKAQAARHALTINWYYADDRGNIGYAHTGFYPRRRPGHDPRLPVPGTGEM
DWLGLLPFSTNPQVYNPRQGFIANWNNQPMRGYPSTDLFAIVWGQADRYA
EIETRLKAMTANGGKVSAQQMWDLIRTTSYADVNRRHFLPFLQRAVQGLP
ADDPRVRLVAGLAAWDGMMTSERQPGYFDNAGPAVMDAWLRAMLRRTLAD
EMPADFFKWYSATGYPTPQAPATGSLNLTTGVKVLFNALAGPEAGVPQRY
DFFNGARADDVILAALDDALAALRQAYGQDPAAWKIPAPPMVFAPKNFLG
VPQADAKAVLCYRATQNRGTENNMTVFDGKSVRAVDVVAPGQSGFVAPDG
TPSPHTRDQFDLYNTFGSKRVWFTADEVRRNATSEETLRYPR

A non-limiting example of a nucleotide sequence encoding SEQ ID NO: 85 is provided by SEQ ID NO: 89:

(SEQ ID NO: 89)
GCGCAACCGGTCGCACCAGCTGCGGGTCAGACCTCCGAGGCGGTAGCTGC
CCGTCCGCAAACCGCAGATGGCAAGGTCACCATCCGTCGCGACGCGTATG
GCATGCCACACGTTTACGCGGACACCGTCTACGGCATTTTCTATGGTTAC
GGCTATGCGGTCGCACAAGATCGCTTGTTCCAGATGGAAATGGCGCGTCG
TTCTACGCAGGGTCGCGTTGCAGAAGTCCTGGGTGCTTCTATGGTGGGCT
TTGACAAGAGCATTCGTGCGAATTTTTCTCCGGAACGCATCCAGCGCCAA
CTGGCTGCGCTGCCTGCAGCTGATCGCCAGGTCCTGGATGGCTACGCAGC
GGGCATGAATGCTTGGCTGGCACGTGTTCGTGCTCAACCGGGTCAGCTGA
TGCCGAAAGAATTTAATGATCTGGGTTTTGCTCCGGCGGATTGGACGGCG
TATGATGTGGCGATGATTTTTGTGGGTACCATGGCTAACCGCTTCTCAGA
CGCCAACAGCGAGATTGATAACCTGGCACTGCTGACCGCCCTGAAAGATC
GTCACGGTGCGGCTGATGCGATGCGCATCTTCAACCAACTGCGTTGGCTG
ACGGATAGCCGTGCTCCGACGACGGTTCCGGCAGAGGCTGGTTCTTACCA
GCCGCCGGTTTTTCAGCCGGATGGTGCAGATCCGCTGGCATACGCGCTGC
CGCGCTATGACGGTACGCCGCCGATGCTGGAGCGTGTGGTGCGCGATCCG
GCAACCCGTGGTGTCGTTGATGGTGCACCGGCAACGCTGCGTGCACAACT
GGCAGCCCAGTACGCACAGTCCGGTCAGCCGGGTATTGCTGGTTTTCCGA
CGACTAGCAACATGTGGATTGTTGGCCGTGACCATGCCAAGGATGCCCGT
AGCATCCTGCTGAATGGTCCGCAGGCCGGTTGGTGGAATCCTGCGTATAC
CTACGGCATTGGTCTGCATGGTGCCGGCTTTGACGTCGTTGGCAACACCC
CTTTTGCTTACCCGAGCATCCTGTTCGGTCATAACGCCCATGTGACTTGG
GGTAGCACTGCCGGTTTCGGCGATGACGTTGATATCTTCGCCGAGAAACT
GGATCCTGCCGACCGTACCCGTTACTTTCACGACGGCCAGTGGAAAACCT
TGGAAAAGCGCACTGATCTGATCTTGGTGAAAGACGCGGCACCAGTCACG
CTGGACGTTTACCGTAGCGTTCACGGTCTGATTGTTAAGTTCGATGATGC
GCAGCACGTCGCATACGCAAAGGCCCGTGCATGGGAAGGCTACGAGCTGC
AGTCCCTGATGGCCTGGACCCGTAAAACCCAGAGCGCGAATTGGGAGCAA
TGGAAAGCGCAAGCAGCTCGCCACGCACTGACGATTAATTGGTATTATGC
AGACGACCGCGGCAACATTGGCTATGCGCACACCGGCTTTTATCCACGTC
GTCGTCCGGGTCACGATCCGCGTTTGCCGGTTCCGGGCACTGGCGAGATG
GACTGGCTGGGTCTGTTGCCATTTTCGACCAACCCGCAAGTCTATAATCC
GCGTCAGGGCTTTATTGCCAATTGGAACAATCAACCGATGCGTGGCTACC
CTAGCACCGATCTGTTCGCTATTGTTTGGGGTCAAGCCGATCGCTATGCC
GAGATCGAAACCCGCCTGAAAGCCATGACGGCGAACGGTGGTAAAGTGAG
CGCGCAACAGATGTGGGACCTGATCCGCACCACGAGCTATGCGGATGTCA
ATCGTCGCCATTTCTTGCCTTTCCTGCAACGTGCCGTCCAAGGCCTGCCG
GCAGATGATCCTCGCGTTCGCCTGGTAGCTGGCCTGGCAGCATGGGACGG
CATGATGACGAGCGAACGCCAACCGGGCTACTTCGATAACGCTGGCCCAG
CCGTTATGGATGCTTGGCTGCGTGCAATGCTGCGCCGCACCTTGGCAGAC
GAGATGCCGGCAGACTTCTTCAAATGGTACAGCGCAACGGGTTATCCGAC
CCCGCAAGCTCCAGCCACTGGTAGCTTGAATCTGACGACGGGCGTTAAAG
TGTTGTTTAATGCGTTGGCCGGTCCTGAGGCTGGTGTGCCGCAGCGCTAC
GACTTCTTCAATGGTGCGCGTGCGGATGACGTCATTTTAGCGGCATTAGA
CGACGCCTTGGCGGCACTGCGTCAGGCGTACGGTCAGGATCCGGCGGCTT
GGAAGATCCCTGCCCCACCGATGGTTTTTGCCCCGAAAAACTTCTTGGGT
GTTCCGCAAGCGGACGCGAAAGCTGTTCTGTGCTATCGCGCGACGCAAAA
TCGTGGTACCGAGAATAATATGACCGTGTTTGACGGCAAGAGCGTCCGCG
CAGTGGATGTTGTCGCTCCGGGCCAATCCGGTTTTGTGGCCCCGGATGGT
ACCCCTTCTCCGCATACTCGTGACCAGTTCGACCTGTACAACACCTTCGG
CAGCAAGCGTGTCTGGTTCACCGCGGATGAGGTTCGTCGTAACGCCACGA
GCGAAGAAACGCTGCGTTATCCGCGCTAA.

In some embodiments, the PGA comprises the sequence of SEQ ID NO: 120 (e.g., amino acids 41 to 863 of SEQ ID NO: 1, comprising in order from N- to C-terminus, an alpha subunit, a spacer, and a beta subunit):

(SEQ ID NO: 120)
QTADGKVTIRRDAYGMPHVYADTVYGIFYGYGYAVAQDRLFQMEMARRST
QGRVAEVLGASMVGFDKSIRANFSPERIQRQLAALPAADRQVLDGYAAGM
NAWLARVRAQPGQLMPKEFNDLGFAPADWTAYDVAMIFVGTMANRFSDAN
SEIDNLALLTALKDRHGAADAMRIFNQLRWLTDSRAPTTVPAEAGSYQPP
VFQPDGADPLAYALPRYDGTPPMLERVVRDPATRGVVDGAPATLRAQLAA
QYAQSGQPGIAGFPTTSNMWIVGRDHAKDARSILLNGPQFGWWNPAYTYG
IGLHGAGFDVVGNTPFAYPSILFGHNAHVTWGSTAGFGDDVDIFAEKLDP
ADRTRYFHDGQWKTLEKRTDLILVKDAAPVTLDVYRSVHGLIVKFDDAQH
VAYAKARAWEGYELQSLMAWIRKTQSANWEQWKAQAARHALTINWYYADD
RGNIGYAHTGFYPRRRPGHDPRLPVPGTGEMDWLGLLPFSINPQVYNPRQ
GFIANWNNQPMRGYPSTDLFAIVWGQADRYAEIETRLKAMTANGGKVSAQ
QMWDLIRTTSYADVNRRHFLPFLQRAVQGLPADDPRVRLVAGLAAWDGMM
TSERQPGYFDNAGPAVMDAWLRAMLRRTLADEMPADFFKWYSATGYPTPQ
APATGSLNLTTGVKVLFNALAGPEAGVPQRYDFFNGARADDVILAALDDA
LAALRQAYGQDPAAWKIPAPPMVFAPKNFLGVPQADAKAVLCYRATQNRG
TENNMTVFDGKSVRAVDVVAPGQSGFVAPDGTPSPHTRDQFDLYNTFGSK
RVWFTADEVRRNATSEETLRYPR

As noted above, different boundaries have been reported between the signal peptide and the alpha subunit, and between the alpha subunit and the spacer.

For example, in some embodiments, the sequence of SEQ ID NO: 1, provided above, includes an alpha subunit, a spacer region and a beta subunit as shown below:

alpha subunit (SEQ ID NO: 5):
AQPVAPAAGQTSEAVAARPQTADGKVTIRRDAYGMPHVYADTVYGIFYGY
GYAVAQDRLFQMEMARRSTQGRVAEVLGASMVGFDKSIRANFSPERIQRQ
LAALPAADRQVLDGYAAGMNAWLARVRAQPGQLMPKEFNDLGFAPADWTA
YDVAMIFVGTMANRFSDANSEIDNLALLTALKDRHGAADAMRIFNQLRWL
TDSRAPTTVPAEAGSYQPPVFQPDGADPLAYALPRYDGTPPMLERVV
spacer (SEQ ID NO: 87):
RDPATRGVVDGAPATLRAQLAAQYAQSGQPGIAGFPTT
beta subunit (SEQ ID NO: 88):
SNMWIVGRDHAKDARSILLNGPQFGWWNPAYTYGIGLHGAGFDVVGNTPF
AYPSILFGHNAHVTWGSTAGFGDDVDIFAEKLDPADRTRYFHDGQWKTLE
KRTDLILVKDAAPVTLDVYRSVHGLIVKFDDAQHVAYAKARAWEGYELQS
LMAWTRKTQSANWEQWKAQAARHALTINWYYADDRGNIGYAHTGFYPRRR
PGHDPRLPVPGTGEMDWLGLLPFSTNPQVYNPRQGFIANWNNQPMRGYPS
TDLFAIVWGQADRYAEIETRLKAMTANGGKVSAQQMWDLIRTTSYADVNR
RHFLPFLQRAVQGLPADDPRVRLVAGLAAWDGMMTSERQPGYFDNAGPAV
MDAWLRAMLRRTLADEMPADFFKWYSATGYPTPQAPATGSLNLTTGVKVL
FNALAGPEAGVPQRYDFENGARADDVILAALDDALAALRQAYGQDPAAWK
IPAPPMVFAPKNFLGVPQADAKAVLCYRATQNRGTENNMTVFDGKSVRAV
DVVAPGQSGFVAPDGTPSPHTRDQFDLYNTFGSKRVWFTADEVRRNATSE
ETLRYPR

In other embodiments, the sequence of SEQ ID NO: 1, provided above, includes an alpha subunit, a spacer region and a beta subunit as shown below:

alpha subunit (SEQ ID NO: 121):
AQPVAPAAGQTSEAVAARPQTADGKVTIRRDAYGMPHVYADTVYGIFYGY
GYAVAQDRLFQMEMARRSTQGRVAEVLGASMVGFDKSIRANFSPERIQRQ
LAALPAADRQVLDGYAAGMNAWLARVRAQPGQLMPKEFNDLGFAPADWTA
YDVAMIFVGTMANRFSDANSEIDNLALLTALKDRHGAADAMRIFNQLRWL
TDSRAPTTVPAEAGSYQPPVFQPDGADPLAY
spacer (SEQ ID NO: 95):
ALPRYDGTPPMLERVVRDPATRGVVDGAPATLRAQLAAQYAQSGQPGIAG
FPTT
beta subunit (as provided in SEQ ID NO: 88)

In other embodiments, the sequence of SEQ ID NO: 1, provided above, includes an alpha subunit, a spacer region and a beta subunit as shown below:

alpha subunit (SEQ ID NO: 127):
AQPVAPAAGQTSEAVAARPQTADGKVTIRRDAYGMPHVYADTVYGIFYGY
GYAVAQDRLFQMEMARRSTQGRVAEVLGASMVGFDKSIRANFSPERIQRQ
LAALPAADRQVLDGYAAGMNAWLARVRAQPGQLMPKEFNDLGFAPADWTA
YDVAMIFVGTMANRFSDANSEIDNLALLTALKDRHGAADAMRIFNQLRWL
TDSRAPTTVPAEAGSYQPPVFQPDGADP
spacer (SEQ ID NO: 97):
LAYALPRYDGTPPMLERVVRDPATRGVVDGAPATLRAQLAAQYAQSGQPG
IAGFPTT
beta subunit (as provided in SEQ ID NO: 88).

In other embodiments, the sequence of SEQ ID NO: 1, provided above, includes an alpha subunit, a spacer region and a beta subunit as shown below:

alpha subunit (SEQ ID NO: 123):
AQPVAPAAGQTSEAVAARPQTADGKVTIRRDAYGMPHVYADTVYGIFYGY
GYAVAQDRLFQMEMARRSTQGRVAEVLGASMVGFDKSIRANFSPERIQRQ
LAALPAADRQVLDGYAAGMNAWLARVRAQPGQLMPKEFNDLGFAPADWTA
YDVAMIFVGTMANRFSDANSEIDNLALLTALKDRHGAADAMRIF
spacer (SEQ ID NO: 99):
NQLRWLTDSRAPTTVPAEAGSYQPPVFQPDGADPLAYALPRYDGTPPMLE
RVVRDPATRGVVDGAPATLRAQLAAQYAQSGQPGIAGFPTT
beta subunit (as provided in SEQ ID NO: 88).

In other embodiments, the sequence of SEQ ID NO: 1, provided above, includes an alpha subunit, a spacer region and a beta subunit as shown below:

alpha subunit (SEQ ID NO: 133):
QTADGKVTIRRDAYGMPHVYADTVYGIFYGYGYAVAQDRLFQMEMARRST
QGRVAEVLGASMVGFDKSIRANFSPERIQRQLAALPAADRQVLDGYAAGM
NAWLARVRAQPGQLMPKEFNDLGFAPADWTAYDVAMIFVGTMANRFSDAN
SEIDNLALLTALKDRHGAADAMRIFNQLRWLTDSRAPTTVPAEAGSYQPP
VFQPDGADPLAYALPRYDGTPPMLERVV
spacer (SEQ ID NO: 87):
RDPATRGVVDGAPATLRAQLAAQYAQSGQPGIAGFPTT
beta subunit (as provided in SEQ ID NO: 88).

In other embodiments, the sequence of SEQ ID NO: 1, provided above, includes an alpha subunit, a spacer region and a beta subunit as shown below:

alpha subunit (SEQ ID NO: 131):
QTADGKVTIRRDAYGMPHVYADTVYGIFYGYGYAVAQDRLFQMEMARRST
QGRVAEVLGASMVGFDKSIRANFSPERIQRQLAALPAADRQVLDGYAAGM
NAWLARVRAQPGQLMPKEFNDLGFAPADWTAYDVAMIFVGTMANRFSDAN
SEIDNLALLTALKDRHGAADAMRIFNQLRWLTDSRAPTTVPAEAGSYQPP
VFQPDGADPLAY
spacer (SEQ ID NO: 95):
ALPRYDGTPPMLERVVRDPATRGVVDGAPATLRAQLAAQYAQSGQPGIAG
FPTT
beta subunit (as provided in SEQ ID NO: 88).

In other embodiments, the sequence of SEQ ID NO: 1, provided above, includes an alpha subunit, a spacer region and a beta subunit as shown below:

alpha subunit (SEQ ID NO: 125):
QTADGKVTIRRDAYGMPHVYADTVYGIFYGYGYAVAQDRLFQMEMARRST
QGRVAEVLGASMVGFDKSIRANFSPERIQRQLAALPAADRQVLDGYAAGM
NAWLARVRAQPGQLMPKEFNDLGFAPADWTAYDVAMIFVGTMANRFSDAN
SEIDNLALLTALKDRHGAADAMRIFNQLRWLTDSRAPTTVPAEAGSYQPP
VFQPDGADP
spacer (SEQ ID NO: 97):
LAYALPRYDGTPPMLERVVRDPATRGVVDGAPATLRAQLAAQYAQSGQPG
IAGFPTT
beta subunit (as provided in SEQ ID NO: 88).

In other embodiments, the sequence of SEQ ID NO: 1, provided above, includes an alpha subunit, a spacer region and a beta subunit as shown below:

alpha subunit (SEQ ID NO: 129):
QTADGKVTIRRDAYGMPHVYADTVYGIFYGYGYAVAQDRLFQMEMARRST
QGRVAEVLGASMVGFDKSIRANFSPERIQRQLAALPAADRQVLDGYAAGM
NAWLARVRAQPGQLMPKEFNDLGFAPADWTAYDVAMIFVGTMANRFSDAN
SEIDNLALLTALKDRHGAADAMRIF
spacer (SEQ ID NO: 99):
NQLRWLTDSRAPTTVPAEAGSYQPPVFQPDGADPLAYALPRYDGTPPMLE
RVVRDPATRGVVDGAPATLRAQLAAQYAQSGQPGIAGFPTT
beta subunit (as provided in SEQ ID NO: 88).

In some embodiments, a PGA of the present disclosure comprises an amino acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to SEQ ID NO: 1, SEQ ID NO: 83, SEQ ID NO: 85, or SEQ ID NO: 120, or any other PGA protein sequence associated with the disclosure. In some embodiments, a PGA of the present disclosure comprises a sequence that is a conservatively substituted version of SEQ ID NO: 1, SEQ ID NO: 83, SEQ ID NO: 85, or SEQ ID NO: 120 or any other PGA protein sequence associated with the disclosure.

In some embodiments, a PGA of the present disclosure is encoded by a nucleic acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to SEQ ID NO: 2 or SEQ ID NO: 84, or any other nucleic acid sequence encoding a PGA associated with the disclosure.

### Alpha and Beta Subunits

In some embodiments, a PGA comprises an alpha subunit and/or a beta subunit. The alpha and beta subunits can be directly linked to each other or can be linked through a spacer or linker region.

In some embodiments, an alpha subunit or a beta subunit is any alpha or beta subunit described in this disclosure.

Sequences of additional alpha and beta subunits of representative PGAs associated with this disclosure are provided in Table 10. It should be appreciated that any of the alpha subunits provided in this disclosure may be combined with any of the beta subunits provided in this disclosure.

In some embodiments, an alpha subunit of a PGA of the present disclosure comprises an amino acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 3-6, 8, 10, 13, 18, 20, or 21. In some embodiments, a PGA of the present disclosure comprises a sequence that is a conservatively substituted version of any one of SEQ ID NOs: 3-6, 8, 10, 13, 18, 20, or 21.

In some embodiments, an alpha subunit of a PGA of the present disclosure is encoded by a nucleic acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 43-46, 48, 50, 51, 53, 58, 60, or 61.

In some embodiments, a beta subunit of a PGA of the present disclosure comprises an amino acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 23-25, 27-32, 34-39, or 88. In some embodiments, a PGA of the present disclosure comprises a sequence that is a conservatively substituted version of any one of SEQ ID NOs: 23-25, 27-32, 34-39, or 88.

In some embodiments, a beta subunit of a PGA of the present disclosure is encoded by a nucleic acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 63-72 or 74-81.

In some embodiments, the alpha subunit comprises a sequence that is at least 90% identical to SEQ ID NO: 3 and the beta subunit comprises a sequence that is at least 90% identical to SEQ ID NO: 23. In some embodiments, the alpha subunit comprises a sequence that is at least 90% identical to SEQ ID NO: 4 and the beta subunit comprises a sequence that is at least 90% identical to SEQ ID NO: 24. In some embodiments, the alpha subunit comprises a sequence that is at least 90% identical to SEQ ID NO: 5 and the beta subunit comprises a sequence that is at least 90% identical to SEQ ID NO: 25. In some embodiments, the alpha subunit comprises a sequence that is at least 90% identical to SEQ ID NO: 6 and the beta subunit comprises a sequence that is at least 90% identical to SEQ ID NO: 23. In some embodiments, the alpha subunit comprises a sequence that is at least 90% identical to SEQ ID NO: 5 and the beta subunit comprises a sequence that is at least 90% identical to SEQ ID NO: 27. In some embodiments, the alpha subunit comprises a sequence that is at least 90% identical to SEQ ID NO: 8 and the beta subunit comprises a sequence that is at least 90% identical to SEQ ID NO: 28. In some embodiments, the alpha subunit comprises a sequence that is at least 90% identical to SEQ ID NO: 5 and the beta subunit comprises a sequence that is at least 90% identical to SEQ ID NO: 29. In some embodiments, the alpha subunit comprises a sequence that is at least 90% identical to SEQ ID NO: 10 and the beta subunit comprises a sequence that is at least 90% identical to SEQ ID NO: 30. In some embodiments, the alpha subunit comprises a sequence that is at least 90% identical to SEQ ID NO: 4 and the beta subunit comprises a sequence that is at least 90% identical to SEQ ID NO: 31. In some embodiments, the alpha subunit comprises a sequence that is at least 90% identical to SEQ ID NO: 5 and the beta subunit comprises a sequence that is at least 90% identical to SEQ ID NO: 32. In some embodiments, the alpha subunit comprises a sequence that is at least 90% identical to SEQ ID NO: 13 and the beta subunit comprises a sequence that is at least 90% identical to SEQ ID NO: 23. In some embodiments, the alpha subunit comprises a sequence that is at least 90% identical to SEQ ID NO: 5 and the beta subunit comprises a sequence that is at least 90% identical to SEQ ID NO: 34. In some embodiments, the alpha subunit comprises a sequence that is at least 90% identical to SEQ ID NO: 4 and the beta subunit comprises a sequence that is at least 90% identical to SEQ ID NO: 35. In some embodiments, the alpha subunit comprises a sequence that is at least 90% identical to SEQ ID NO: 4 and the beta subunit comprises a sequence that is at least 90% identical to SEQ ID NO: 36. In some embodiments, the alpha subunit comprises a sequence that is at least 90% identical to SEQ ID NO: 5 and the beta subunit comprises a sequence that is at least 90% identical to SEQ ID NO: 37. In some embodiments, the alpha subunit comprises a sequence that is at least 90% identical to SEQ ID NO: 18 and the beta subunit comprises a sequence that is at least 90% identical to SEQ ID NO: 38. In some embodiments, the alpha subunit comprises a sequence that is at least 90% identical to SEQ ID NO: 5 and the beta subunit comprises a sequence that is at least 90% identical to SEQ ID NO: 39. In some embodiments, the alpha subunit comprises a sequence that is at least 90% identical to SEQ ID NO: 20 and the beta subunit comprises a sequence that is at least 90% identical to SEQ ID NO: 28. In some embodiments, the alpha subunit comprises a sequence that is at least 90% identical to SEQ ID NO: 21 and the beta subunit comprises a sequence that is at least 90% identical to SEQ ID NO: 30. In some embodiments, the alpha subunit comprises a sequence that is at least 90% identical to SEQ ID NO: 18 and the beta subunit comprises a sequence that is at least 90% identical to SEQ ID NO: 30.

In some embodiments, the alpha subunit comprises the sequence of SEQ ID NO: 3 and the beta subunit comprises the sequence of SEQ ID NO: 23. In some embodiments, the alpha subunit comprises the sequence of SEQ ID NO: 4 and the beta subunit comprises the sequence of SEQ ID NO: 24. In some embodiments, the alpha subunit comprises the sequence of SEQ ID NO: 5 and the beta subunit comprises the sequence of SEQ ID NO: 25. In some embodiments, the alpha subunit comprises the sequence of SEQ ID NO: 6 and the beta subunit comprises the sequence of SEQ ID NO: 23. In some embodiments, the alpha subunit comprises the sequence of SEQ ID NO: 5 and the beta subunit comprises the sequence of SEQ ID NO: 27. In some embodiments, the alpha subunit comprises the sequence of SEQ ID NO: 8 and the beta subunit comprises the sequence of SEQ ID NO: 28. In some embodiments, the alpha subunit comprises the sequence of SEQ ID NO: 5 and the beta subunit comprises the sequence of SEQ ID NO: 29. In some embodiments, the alpha subunit comprises the sequence of SEQ ID NO: 10 and the beta subunit comprises the sequence of SEQ ID NO: 30. In some embodiments, the alpha subunit comprises the sequence of SEQ ID NO: 4 and the beta subunit comprises the sequence of SEQ ID NO: 31. In some embodiments, the alpha subunit comprises the sequence of SEQ ID NO: 5 and the beta subunit comprises the sequence of SEQ ID NO: 32. In some embodiments, the alpha subunit comprises the sequence of SEQ ID NO: 13 and the beta subunit comprises the sequence of SEQ ID NO: 23. In some embodiments, the alpha subunit comprises the sequence of SEQ ID NO: 5 and the beta subunit comprises the sequence of SEQ ID NO: 34. In some embodiments, the alpha subunit comprises the sequence of SEQ ID NO: 4 and the beta subunit comprises the sequence of SEQ ID NO: 35. In some embodiments, the alpha subunit comprises the sequence of SEQ ID NO: 4 and the beta subunit comprises the sequence of SEQ ID NO: 36. In some embodiments, the alpha subunit comprises the sequence of SEQ ID NO: 5 and the beta subunit comprises the sequence of SEQ ID NO: 37. In some embodiments, the alpha subunit comprises the sequence of SEQ ID NO: 18 and the beta subunit comprises the sequence of SEQ ID NO: 38. In some embodiments, the alpha subunit comprises the sequence of SEQ ID NO: 5 and the beta subunit comprises the sequence of SEQ ID NO: 39. In some embodiments, the alpha subunit comprises the sequence of SEQ ID NO: 20 and the beta subunit comprises the sequence of SEQ ID NO: 28. In some embodiments, the alpha subunit comprises the sequence SEQ ID NO: 21 and the beta subunit comprises the sequence of SEQ ID NO: 30. In some embodiments, the alpha subunit comprises the sequence of SEQ ID NO: 18 and the beta subunit comprises the sequence of SEQ ID NO: 30.

In some embodiments, an alpha subunit comprises a region of a PGA that corresponds to amino acid residue positions 22-268 in SEQ ID NO: 1. In some embodiments, an alpha subunit comprises a region of a PGA that corresponds to amino acid residue positions 41-215 in SEQ ID NO: 1. In some embodiments, a PGA as disclosed in the present application comprises an alpha subunit that is at least 70%, 75%, 80%, 85%, 90%, 95%, 96%, 97%, 98%, 99%, or 100% identical to a sequence comprising amino acid residue positions 22-268 or 41-215 in SEQ ID NO: 1.

In some embodiments, a PGA comprises an alpha subunit as described in this disclosure and a beta subunit from a different species or source. In some embodiments, a PGA comprises a beta subunit as described in this disclosure and an alpha subunit from a different species or source. PGA variants comprising an alpha and a beta subunit from different species or sources are described in Mayer et al. Appl. Microbiol. Biotechnol. 2019 Jun. 21. pii: 10.1007/s00253-019-09977-8, which is incorporated by reference in this disclosure in its entirety.

### Spacer Sequences

Alpha and beta subunits of a PGA may be connected to each other by a spacer sequence. As used in this disclosure, a “spacer sequence,” a “spacer,” a “linker,” and a “linker sequence,” which are used interchangeably, refer to a peptide sequence that occurs between and connects two protein domains or subunits.

In some embodiments, the spacer is absent from the PGA protein, and the PGA protein comprises an alpha subunit and a beta subunit. In some embodiments, the spacer is absent from the PGA protein, as the spacer has been cleaved at both cleavage sites between the C-terminus of the alpha subunit and the N-terminus of the spacer; and between the C-terminus of the spacer and the N-terminus of the beta subunit. In some embodiments, the spacer is present in the PGA and is interposed between the alpha subunit and the beta subunit. In some embodiments, the amino acid sequence of the spacer is or comprises the sequence of amino acids interposed between the alpha subunit and the beta subunit.

One of ordinary skill in the art would be able to design an appropriate spacer sequence. In some embodiments, a spacer sequence is a wild-type sequence from a naturally occurring protein. In some embodiments, a spacer sequence is a variant sequence. In some embodiments, a spacer sequence is a synthetic sequence. In some embodiments, a spacer sequence is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 amino acids in length. In some embodiments, a spacer sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, a spacer sequence is 1-50, 5-45, 10-40, 15-35, or 20-30 amino acids in length. In some embodiments, a spacer sequence is 38 amino acids in length. In some embodiments, a spacer sequence comprises a sequence that is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical, including all values or ranges in between, to any one of SEQ ID NOs: 87, 97 or 99. In some embodiments, a spacer sequence comprises a sequence that is at least 60%, 65%, 70%, 75%, 80%, 85%, 90%, 95%, 96%, 97%, 98%, 99% or 100% identical to any one of SEQ ID NOs: 87, 97 or 99. In some embodiments, a spacer sequence comprises no more than 1, 2, 3, 4, 5, 6, 7, 8, 9, 10, 11, 12, 13, 14, 15, 16, 17, 18, 19, or 20 amino acid differences from the sequence of any one of SEQ ID NOs: 87, 97 or 99.

In some embodiments, the sequence of the spacer is or comprises all or a portion of a wild-type sequence between the alpha and the beta subunit of a PGA. In various embodiments, the amino acid sequence of the spacer, because it lies between the alpha and beta subunits, is the sequence which lies between the C-terminus of the alpha subunit and the N-terminus of the beta subunit. Thus, the amino acid numbering of the boundaries of the spacer sequence may vary depending on the amino acid numbering of the C-terminus of the alpha subunit and/or the amino acid numbering of the N-terminus of the beta subunit.

In some embodiments, the sequence of the beta subunit has an N-terminus at position 307 [and thus the sequence of the beta subunit begins with SNMWIVGRDHAK (SEQ ID NO: 90)]. If the N-terminus of the beta subunit is at position 307, then the C-terminus of the spacer ends with the sequence . . . SGQPGIAGFPTT (SEQ ID NO: 91). In various embodiments, the definition of the sequence of the spacer is the sequence of amino acids which lies between the C-terminus of the alpha subunit (e.g., at position 268, 252, 249, or 215) and the N-terminus of the beta subunit. In various embodiments, the definition of the sequence of the spacer is the sequence of amino acids which lies between the C-terminus of the alpha subunit (e.g., at position 268, 252, 249, or 215) and the N-terminus of the beta subunit, which is at position 307. In some embodiments, if the alpha subunit C-terminus is at position 268 [e.g., the alpha subunit ends with the sequence . . . DAMRIFNQLR WLTDSRAPTT VPAEAGSYQP PVFQPDGADP LAYALPRYDG TPPMLERVV (SEQ ID NO: 92)], then the sequence of the spacer can be or comprise: RDPATRGVVD GAPATLRAQL AAQYAQSGQP GIAGFPTT (SEQ ID NO: 87). See, for example: WO2021096298A1. In some embodiments, if the alpha subunit C-terminus is at position 252 [e.g., the alpha subunit ends with the sequence . . . DAMRIFNQLR WLTDSRAPTT VPAEAGSYQP PVFQPDGADP LAY ((SEQ ID NO: 94)], then the sequence of the spacer can be or comprise: ALPRYDGTPP MLERVVRDPA TRGVVDGAPA TLRAQLAAQY AQSGQPGIAG FPTT (SEQ ID NO: 95). See, for example: CN105274082B.

In some embodiments, if the alpha subunit C-terminus is at position 249 [e.g., the alpha subunit ends with the sequence . . . DAMRIFNQLR WLTDSRAPTT VPAEAGSYQP PVFQPDGADP (SEQ ID NO: 96)], then the sequence of the spacer can be or comprise: LAYALPRYDG TPPMLERVVR DPATRGVVDG APATLRAQLA AQYAQSGQPG IAGFPTT (SEQ ID NO: 97). See, for example: IN202021000782A or WO2021140526A1. In some embodiments, if the alpha subunit C-terminus is at position 215 [e.g., the alpha subunit ends with the sequence . . . . DAMRIF (SEQ ID NO: 98)], then the sequence of the spacer can be or comprise: NQLRWLTDSR APTTVPAEAG SYQPPVFQPD GADPLAYALP RYDGTPPMLE RVVRDPATRG VVDGAPATLR AQLAAQYAQSG QPGIAGFPTT (SEQ ID NO: 99)]. See, for example: KR101985911B1 or IN383213B. In various embodiments, the sequence of the spacer can be or can comprise the sequence of any spacer or peptide linker known in the art. In some embodiments, a spacer can comprise a single amino acid or up to 100 or more amino acids.

In some embodiments, a spacer sequence comprises a sequence that is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical, including all values or ranges in between, to any one of SEQ ID NOs: 87, 95, 97, or 99:

(SEQ ID NO: 87)
RDPATRGVVDGAPATLRAQLAAQYAQSGQPGIAGFPTT;
(SEQ ID NO: 95)
ALPRYDGTPPMLERVVRDPATRGVVDGAPATLRAQLAAQYAQSGQPGIAG
FPTT;
(SEQ ID NO: 97)
LAYALPRYDGTPPMLERVVRDPATRGVVDGAPATLRAQLAAQYAQSGQPG
IAGFPTT;
(SEQ ID NO: 99)
NQLRWLTDSRAPTTVPAEAGSYQPPVFQPDGADPLAYALPRYDGTPPMLE
RVVRDPATRGVVDGAPATLRAQLAAQYAQSGQPGIAGFPTT.

In some embodiments, a spacer sequence comprises a sequence that is at least 60%, 65%, 70%, 75%, 80%, 85%, 90%, 95%, 96%, 97%, 98%, 99% or 100% identical to any one of SEQ ID NOs: 87, 95, 97, or 99:

(SEQ ID NO: 87)
RDPATRGVVDGAPATLRAQLAAQYAQSGQPGIAGFPTT;
(SEQ ID NO: 95)
ALPRYDGTPPMLERVVRDPATRGVVDGAPATLRAQLAAQYAQSGQPGIAG
FPTT;
(SEQ ID NO: 97)
LAYALPRYDGTPPMLERVVRDPATRGVVDGAPATLRAQLAAQYAQSGQPG
IAGFPTT;
(SEQ ID NO: 99)
NQLRWLTDSRAPTTVPAEAGSYQPPVFQPDGADPLAYALPRYDGTPPMLE
RVVRDPATRGVVDGAPATLRAQLAAQYAQSGQPGIAGFPTT.

In some embodiments, the sequence of the spacer can be the sequence of a spacer from a homologous PGA (e.g., a PGA which is not from an Achromobacter).

In some embodiments, the amino acid sequence of a spacer can be or can comprise the amino acid sequence of any spacer known in the art, including but not limited to: SR (SEQ ID NO: 100); SSR (SEQ ID NO: 101); RRS (SEQ ID NO: 102); SSG (SEQ ID NO: 103); G (SEQ ID NO: 104); GGGGS (SEQ ID NO: 105); GGGSS (SEQ ID NO: 106); GGGGSGGGS (SEQ ID NO: 107); GSGGGSGGGGS GGGGS (SEQ ID NO: 108); (A), (SEQ ID NO: 109), (GA) n (SEQ ID NO: 110), or (GGGGS)ₙ (SEQ ID NO: 111) where n is independently 1-50; KG (SEQ ID NO: 112); LLGAAAKGAA AKGSAA (SEQ ID NO: 113); LLGGGGSGGG GSAAAGSAA (SEQ ID NO: 114); MNHLVHHHHH HIEGRHMELGTLEGS (SEQ ID NO: 115); MHHHHHHKH (SEQ ID NO: 116); or GLNDIFEAQKIEWHE (SEQ ID NO: 117).

In some embodiments a spacer is rigid. In other embodiments, a spacer is flexible. In some embodiments, a spacer is a hinge-like region (such as described in and incorporated by reference from WO 94/04678). Various spacers have been described in, for example: Chaudhary et al. 1989 Nature 339:394-397; and U.S. Pat. No. 10,060,920B2; WO 99/42077; Roux, et al. 1998 J. Immunol. 161:4083, which are incorporated by reference in this disclosure in their entireties. In some embodiments, a spacer has the amino acid sequence represented by G1-X1-Y1-G2-X2-Y2 (wherein G1 and G2 represent glycine; and X1, Y1, X2, and Y2 may be same or different and each represent an amino acid residue). In some embodiments a spacer is a helix-forming H-spacer. See, for example: Arai et al. 2001 Prot. Engin. 14:529-532; Marqusee et al. 1987 Proc. Natl. Acad. Sci. USA 84:8898, which are incorporated by reference in this disclosure in their entireties. In some embodiments a spacer is a F-spacer. See, for example: Arai et al. 2001 Prot. Engin. 14:529-532; Alfthan et al. 1995 Prot. Engin. 8:725-731, which are incorporated by reference in this disclosure in their entireties.

In some embodiments, a spacer has a sequence that does not interfere with cleavage of the spacer. Without wishing to be bound by any particular theory, the PGA of Achromobacter is reported to undergo autoproteolysis to remove the spacer; see, for example, KR101985911B1 and WO2021096298A1, which are incorporated by reference in this disclosure in their entireties. As one of ordinary skill in the art would appreciate, any spacer sequence under consideration for interposition between the alpha and beta subunits of a PGA can be readily tested to determine whether the spacer is capable of being cleaved (e.g., by autoproteolysis or otherwise).

### Signal Peptides

Any of the PGAs described in this application, including variant PGAs, may comprise a signal peptide. Signal peptides, also referred to as “signal sequences,” generally comprise approximately 15-50 amino acids and can be involved in regulating trafficking of a newly translated protein to a particular cellular compartment and/or the cellular secretory pathway. A signal peptide may be located N-terminal or C-terminal relative to a PGA associated with the disclosure. A PGA may be linked to one or more signal peptides. In some embodiments, a PGA may be linked to one or more signal peptides at the N-terminus and one or more signal peptides at the C-terminus.

A signal peptide that is located at the N-terminus of a PGA (e.g., at the N-terminus of SEQ ID NO: 83) may comprise a methionine at the N-terminus of the signal peptide. In some embodiments, a methionine is added to a signal peptide if the signal peptide will be located at the N-terminus of a PGA. In some embodiments, a signal peptide that is normally associated with a PGA (e.g., a naturally occurring signal peptide that is present in a naturally occurring PGA) may be removed or replaced with one or more different signal peptides.

In some embodiments, the sequence of the signal peptide is or comprises the sequence of amino acids 1 to 21 of SEQ ID NO: 1 (e.g., SEQ ID NO: 118).

In some embodiments, the sequence of the signal peptide is or comprises the sequence of amino acids 1 to 40 of SEQ ID NO: 1 (e.g., SEQ ID NO: 119).

In some embodiments, the signal peptide is a peptide sequence derived from an Achromobacter PGA protein (e.g., a homolog of a PGA of SEQ ID NO: 1 that is from an Achromobacter which is not Achromobacter sp. CCM 4824). In some embodiments, the signal peptide is a peptide sequence derived from an Achromobacter protein which is not PGA. In some embodiments, the signal peptide is a peptide sequence derived from a homolog of PGA which is not from Achromobacter. In some embodiments, the signal peptide is a peptide sequence derived from a protein or peptide which is neither a PGA nor from Achromobacter.

In some embodiments, the signal peptide is a peptide sequence derived from a particular protein (e.g., is similar or identical to or comprises a sequence similar or identical to a signal peptide sequence from that protein). In some embodiments, the signal peptide is a peptide sequence derived from an Achromobacter protein. In some embodiments, the signal peptide is derived from an Achromobacter pulmonis, Achromobacter sp. CCM 4824, Achromobacter xylosoxidans, Achromobacter deleyi, Achromobacter ruhlandii, Achromobacter sp. 2789STDY5608621, Achromobacter dolens, Achromobacter denitrificans, Achromobacter sp., Achromobacter insuavis, or Achromobacter sp. 2789STDY5608615 protein. In some embodiments, the signal peptide is derived from an Achromobacter protein associated with GenBank Accession No. WP_054416041.1. In some embodiments, the signal peptide is derived from an Achromobacter protein associated with GenBank Accession No. WP_054416041.1; WP_064523634.1; WP_054432140.1; WP_049074600.1; or WP_110135135.1. In some embodiments, the signal peptide is derived from an Achromobacter deleyi protein associated with GenBank Accession No. WP_198484457.1. In some embodiments, the signal peptide is derived from an Achromobacter denitrificans protein associated with GenBank Accession No. WP_059269957.1. In some embodiments, the signal peptide is derived from an Achromobacter dolens protein associated with GenBank Accession No. WP_175168123.1; WP_054504077.1; WP_269874666.1; or WP_175188912.1. In some embodiments, the signal peptide is derived from an Achromobacter insuavis protein associated with GenBank Accession No. WP_241070234.1; WP_006394292.1; WP_238891355.1; WP_116521627.1; WP_241075078.1; WP_180188618.1; or WP_241133889.1. In some embodiments, the signal peptide is derived from an Achromobacter pulmonis protein associated with GenBank Accession No. WP_175131820.1. In some embodiments, the signal peptide is derived from an Achromobacter ruhlandii protein associated with GenBank Accession No. WP_238923440.1; WP_175183301.1; WP_238908674.1; MCI1839532.1; WP_063580820.1; WP_269888873.1; WP_063588022.1; WP_063583887.1; WP_175146691.1; WP_100508242.1; WP_175203347.1; WP_169537587.1; WP_263900605.1; or WP_100502481.1. In some embodiments, the signal peptide is derived from an Achromobacter sp. protein associated with GenBank Accession No. MCG2600797.1. In some embodiments, the signal peptide is derived from an Achromobacter sp. 2789STDY5608615 protein associated with GenBank Accession No. WP_054475843.1. In some embodiments, the signal peptide is derived from an Achromobacter sp. 2789STDY5608621 protein associated with GenBank Accession No. WP_054489548.1. In some embodiments, the signal peptide is derived from an Achromobacter sp. CCM 4824 protein associated with GenBank Accession No. AAY25991.1. In some embodiments, the signal peptide is derived from an Achromobacter xylosoxidans protein associated with GenBank Accession No. WP_241144499.1; WP_076410961.1; WP_061071629.1; WP_241055367.1; WP_269861849.1; OCZ97543.1; WP_068951300.1; WP_155872742.1; WP_240682051.1; WP_241064350.1; WP_241120594.1; WP_054446873.1; WP_054514125.1; WP_148316728.1; WP_020927232.1; WP_241131711.1; WP_076468850.1; WP_047992836.1; WP_269877707.1; WP_241117868.1; WP_241083246.1; WP_054448687.1; WP_173010931.1; WP_054482473.1; WP_163108633.1; WP_155865759.1; WP_054472518.1; WP_054515796.1; WP_058664188.1; WP_054471062.1; WP_049055580.1; WP_053498043.1; WP_150100272.1; WP_054485724.1; WP_149895007.1; MCH1994709.1; WP_054438878.1; WP_269888026.1; WP_251200273.1; WP_173001107.1; WP_054518177.1; WP_024068562.1; WP_026383636.1; WP_240294413.1; WP_238878048.1; WP_104414216.1; WP_200221158.1; WP_054505584.1; WP_240680917.1; WP_241077040.1; WP_006387043.1; WP_241123126.1; WP_238920387.1; WP_246892410.1; WP_054508119.1; WP_246893238.1; WP_107316525.1; WP_155868350.1; WP_241072057.1; WP_060721457.1; WP_238927272.1; AVC04856.1; or WP_054499894.1.

In some embodiments, the signal peptide is a peptide sequence that is artificial.

### PGA Variants

Naturally occurring PGAs that produce beta-lactams generally produce a mixture of beta-lactams and undesired by-products. For example, a PGA can catalyze a reaction converting 6-APA and D-HPGM to D-amoxicillin and/or 7-ADCA and D-PGM to cephalexin. In addition, as described in Example 1, a PGA can synthesize a desired product D-amoxicillin or it can hydrolyze the D-HPGM to produce the undesired by-product D-HPG.

It would be advantageous to be able to influence the product of a PGA-catalyzed reaction to shift production to beta-lactams, instead of undesired by-products. In particular, given the importance of beta-lactam antibiotics in human health, it would be advantageous to be able to produce increased amounts of beta-lactams. Aspects of the disclosure relate to the surprising identification of PGA variants that produce increased amounts of beta-lactams and/or more beta-lactam products compared to undesired by-products, compared to that produced by a control enzyme. As shown in Examples 1-4, it was surprisingly found that PGA variants that included amino acid substitutions relative to the sequence of SEQ ID NO: 1 produced more beta-lactam product than undesired by-product and did so at a higher synthesis rate.

In some embodiments, the sequence of a PGA associated with the disclosure comprises one or more amino acid substitutions relative to the sequence of SEQ ID NO: 1, wherein the one or more amino acid substitutions are at one or more positions corresponding to position T90, T181, M182, R185, A189, N326, F330, W333, L362, D380, T482, H498, V563 and/or L770 in SEQ ID NO: 1.

In some embodiments, a PGA comprises M182N and F330A substitutions relative to the sequence of SEQ ID NO: 1. In some embodiments, a PGA comprises F330A and W333Y substitutions relative to the sequence of SEQ ID NO: 1. In some embodiments, a PGA comprises T90V, A189V, F330A, W333I, and D380V substitutions relative to the sequence of SEQ ID NO: 1. In some embodiments, a PGA comprises T90V, A189V, F330A, D380V, H498L, and V563Q substitutions relative to the sequence of SEQ ID NO: 1. In some embodiments, a PGA comprises M182L, F330A, and T482P substitution relative to the sequence of SEQ ID NO: 1. In some embodiments, a PGA comprises T90V, A189V, F330A, W333I, D380I, and H498L substitutions relative to the sequence of SEQ ID NO: 1. In some embodiments, a PGA comprises M182T, F330A, and L362F substitutions relative to the sequence of SEQ ID NO: 1. In some embodiments, a PGA comprises M182L, R185F, F330A, and T482P substitutions, relative to the sequence of SEQ ID NO: 1. In some embodiments, a PGA comprises an F330G substitution relative to the sequence of SEQ ID NO: 1. In some embodiments, a PGA comprises M182I and F330A substitutions relative to the sequence of SEQ ID NO: 1. In some embodiments, a PGA comprises R185F, N326A, F330A, and T482P substitutions, relative to the sequence of SEQ ID NO: 1. In some embodiments, a PGA comprises F330A and W333P substitutions relative to the sequence of SEQ ID NO: 1. In some embodiments, a PGA comprises F330A and L770T substitutions relative to the sequence of SEQ ID NO: 1. In some embodiments, a PGA comprises T181M and F330A substitutions relative to the sequence of SEQ ID NO: 1. In some embodiments, a PGA comprises F330A and D380Q substitutions relative to the sequence of SEQ ID NO: 1. In some embodiments, a PGA comprises F330A and W333I substitutions relative to the sequence of SEQ ID NO: 1. In some embodiments, a PGA comprises R185F, F330A, and T482P substitutions relative to the sequence of SEQ ID NO: 1. In some embodiments, a PGA comprises F330A and L770A substitutions relative to the sequence of SEQ ID NO: 1. In some embodiments, a PGA comprises T90V, A189V, F330A, W333T, D380V, and H498S substitutions relative to the sequence of SEQ ID NO: 1. In some embodiments, a PGA comprises T181M, M182F, F330A, and L362F substitutions relative to the sequence of SEQ ID NO: 1.

In some embodiments, a PGA comprises an alanine (A) at a residue corresponding to residue 330 in SEQ ID NO: 1. In some embodiments, a PGA comprises: valine (V) at a residue corresponding to residue 90 in SEQ ID NO: 1; methionine (M) at a residue corresponding to residue 181 in SEQ ID NO: 1; asparagine (N) at a residue corresponding to residue 182 in SEQ ID NO: 1; leucine (L) at a residue corresponding to residue 182 in SEQ ID NO: 1; threonine (T) at a residue corresponding to residue 182 in SEQ ID NO: 1; isoleucine (I) at a residue corresponding to residue 182 in SEQ ID NO: 1; phenylalanine (F) at a residue corresponding to residue 182 in SEQ ID NO: 1; F at a residue corresponding to residue 185 in SEQ ID NO: 1; V at a residue corresponding to residue 189 in SEQ ID NO: 1; A at a residue corresponding to residue 326 in SEQ ID NO: 1; A at a residue corresponding to residue 330 in SEQ ID NO: 1; glycine (G) at a residue corresponding to residue 330 in SEQ ID NO: 1; tyrosine (Y) at a residue corresponding to residue 333 in SEQ ID NO: 1; I at a residue corresponding to residue 333 in SEQ ID NO: 1; proline (P) at a residue corresponding to residue 333 in SEQ ID NO: 1; T at a residue corresponding to residue 333 in SEQ ID NO: 1; F at a residue corresponding to residue 362 in SEQ ID NO: 1; glutamine (Q) at a residue corresponding to residue 380 in SEQ ID NO: 1; I at a residue corresponding to residue 380 in SEQ ID NO: 1; V at a residue corresponding to residue 380 in SEQ ID NO: 1; P at a residue corresponding to residue 482 in SEQ ID NO: 1; S at a residue corresponding to residue 498 in SEQ ID NO: 1; L at a residue corresponding to residue 498 in SEQ ID NO: 1; Q at a residue corresponding to residue 563 in SEQ ID NO: 1; T at a residue corresponding to residue 770 in SEQ ID NO: 1; and/or A at a residue corresponding to residue 770 in SEQ ID NO: 1.

In some embodiments, an alpha subunit of a PGA enzyme comprises an amino acid substitution relative to the sequence of SEQ ID NO: 1. In some embodiments, an alpha subunit of a PGA comprises a substitution mutation at a residue corresponding to residue 90 in SEQ ID NO: 1; at a residue corresponding to residue 181 in SEQ ID NO: 1; at a residue corresponding to residue 182 in SEQ ID NO: 1; at a residue corresponding to residue 185 in SEQ ID NO: 1; and/or V at a residue corresponding to residue 189 in SEQ ID NO: 1. In some embodiments, an alpha subunit of a PGA enzyme comprises a V at a residue corresponding to residue 90 in SEQ ID NO: 1; M at a residue corresponding to residue 181 in SEQ ID NO: 1; N at a residue corresponding to residue 182 in SEQ ID NO: 1; L at a residue corresponding to residue 182 in SEQ ID NO: 1; T at a residue corresponding to residue 182 in SEQ ID NO: 1; I at a residue corresponding to residue 182 in SEQ ID NO: 1; F at a residue corresponding to residue 182 in SEQ ID NO: 1; F at a residue corresponding to residue 185 in SEQ ID NO: 1; and/or V at a residue corresponding to residue 189 in SEQ ID NO: 1.

In some embodiments, a beta subunit of a PGA comprises an amino acid substitution relative to the sequence of SEQ ID NO: 1. In some embodiments, a beta subunit of a PGA comprises an amino acid substitution at a residue corresponding to residue 326 in SEQ ID NO: 1; at a residue corresponding to residue 330 in SEQ ID NO: 1; at a residue corresponding to residue 333 in SEQ ID NO: 1; at a residue corresponding to residue 333 in SEQ ID NO: 1; at a residue corresponding to residue 362 in SEQ ID NO: 1; at a residue corresponding to residue 380 in SEQ ID NO: 1; at a residue corresponding to residue 482 in SEQ ID NO: 1; at a residue corresponding to residue 498 in SEQ ID NO: 1; at a residue corresponding to residue 563 in SEQ ID NO: 1; and/or at a residue corresponding to residue 770 in SEQ ID NO: 1. In some embodiments, a beta subunit of a PGA comprises an A at a residue corresponding to residue 326 in SEQ ID NO: 1; A at a residue corresponding to residue 330 in SEQ ID NO: 1; G at a residue corresponding to residue 330 in SEQ ID NO: 1; Y at a residue corresponding to residue 333 in SEQ ID NO: 1; I at a residue corresponding to residue 333 in SEQ ID NO: 1; P at a residue corresponding to residue 333 in SEQ ID NO: 1; T at a residue corresponding to residue 333 in SEQ ID NO: 1; F at a residue corresponding to residue 362 in SEQ ID NO: 1; Q at a residue corresponding to residue 380 in SEQ ID NO: 1; I at a residue corresponding to residue 380 in SEQ ID NO: 1; V at a residue corresponding to residue 380 in SEQ ID NO: 1; P at a residue corresponding to residue 482 in SEQ ID NO: 1; S at a residue corresponding to residue 498 in SEQ ID NO: 1; L at a residue corresponding to residue 498 in SEQ ID NO: 1; Q at a residue corresponding to residue 563 in SEQ ID NO: 1; T at a residue corresponding to residue 770 in SEQ ID NO: 1; and/or A at a residue corresponding to residue 770 in SEQ ID NO: 1.

TABLE 1
Representative amino acid substitutions in PGA variants

| Position in | | | | | |
|---|---|---|---|---|---|
| SEQ ID NO: | Amino Acid Substitutions | | | | |
| 1 | Relative to SEQ ID NO: 1 | | | | |
| T90 | V | | | | |
| T181 | M | | | | |
| M182 | F | I | L | N | T |
| R185 | F | | | | |
| A189 | V | | | | |
| N326 | A | | | | |
| F330 | A | G | | | |
| W333 | I | P | T | Y | |
| L362 | F | | | | |
| D380 | I | Q | V | | |
| T482 | P | | | | |
| H498 | L | S | | | |
| V563 | Q | | | | |
| L770 | A | T | | | |

In the above tables and other annotations in this disclosure, the first letter indicates the amino acid naturally occurring at a particular position in a reference sequence, and the number indicates the amino acid position in the reference sequence (e.g., SEQ ID NO: 1). For example, in the first row in Table 1, T90 indicates that a Threonine is normally at position 90 in the sequence of reference sequence SEQ ID NO: 1. In the amino acid substitution indicated in the first row in Table 1, T is replaced by V (Valine).

Variants of enzymes described in this disclosure (e.g., PGA enzymes, including variants of polynucleotide and polypeptide sequences) are encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Aspects of the disclosure related to identification of PGA variants with improved properties. In some embodiments, a PGA associated with the disclosure is able to convert a precursor to a beta-lactam at a higher rate than a positive control PGA. In some embodiments, a PGA associated with the disclosure exhibits a higher amoxicillin or cephalexin synthesis rate than a positive control PGA. In some embodiments, a positive control PGA has the same sequence as the PGA variant except for one or more amino acid substitutions present in the variant PGA, as described in the present disclosure/Examples. In some embodiments, a PGA can be used to produce a variety of different beta-lactam antibiotics (including, but not limited, to those described herein or known in the art).

Amoxicillin synthesis rate refers to the amount of amoxicillin produced by the conversion of 6-aminopenicillanic acid (6-APA) and D-4-hydroxyphenylglycine methyl ester (D-HPGM) to amoxicillin by a PGA per hour, measured as μM per hour (μM/h). Cephalexin synthesis rate refers to the amount of cephalexin produced by the conversion of 7-aminodeacetoxycephalosporanic acid (7-ADCA) and D-phenylglycine methyl ester (D-PGM) to cephalexin by a PGA per hour, measured as μM per hour (μM/h). “Rate improvement” refers to an increase in amoxicillin or cephalexin synthesis rate by a PGA variant, as compared to a positive control PGA. In some embodiments, the synthesis rate of the PGA variant compared to the synthesis rate of the positive control PGA is improved by at least 1%, at least 5%, at least 10%, at least 15%, at least 20%, at least 25%, at least 30%, at least 40%, at least 50%, at least 60%, at least 70%, at least 80%, at least 90%, at least 100%, at least 150%, at least 200%, or at least 500%.

In some embodiments, a PGA that exhibits rate improvement (e.g., improved amoxicillin synthesis rate) comprises: a V at a residue corresponding to residue 90 in SEQ ID NO: 1; a V at a residue corresponding to residue 189 in SEQ ID NO: 1; an A or G at a residue corresponding to residue 330 in SEQ ID NO: 1; an I, P, T, or Y at a residue corresponding to residue 333 in SEQ ID NO: 1; an I, Q, or V at a residue corresponding to residue 380 in SEQ ID NO: 1; an A or G at a residue corresponding to residue 330 in SEQ ID NO: 1; an L or S at a residue corresponding to residue 498 in SEQ ID NO: 1; a Q at a residue corresponding to residue 563 in SEQ ID NO: 1; and/or an A or T at a residue corresponding to residue 770 in SEQ ID NO: 1.

In some embodiments, a PGA with improved amoxicillin synthesis properties compared to a control PGA comprises: F330A and W333Y substitutions relative to the sequence of SEQ ID NO: 1; T90V, A189V, F330A, W333I, and D380V substitutions relative to the sequence of SEQ ID NO: 1; T90V, A189V, F330A, D380V, H498L, and V563Q substitutions relative to the sequence of SEQ ID NO: 1; T90V, A189V, F330A, W333I, D380I, and H498L substitutions relative to the sequence of SEQ ID NO: 1; an F330G substitution relative to the sequence of SEQ ID NO: 1; F330A and W333P substitutions relative to the sequence of SEQ ID NO: 1; F330A and L770T substitutions relative to the sequence of SEQ ID NO: 1; F330A and D380Q substitutions relative to the sequence of SEQ ID NO: 1; F330A and W333I substitutions relative to the sequence of SEQ ID NO: 1; F330A and L770A substitutions relative to the sequence of SEQ ID NO: 1; or T90V, A189V, F330A, W333T, D380V, and H498S substitutions relative to the sequence of SEQ ID NO: 1.

In some embodiments, a host cell capable of producing amoxicillin expresses a PGA that comprises F330A and W333I substitutions relative to the sequence of SEQ ID NO: 1. In some embodiments, a PGA with improved amoxicillin synthesis properties compared to a control PGA comprises F330A and W333I substitutions relative to the sequence of SEQ ID NO: 1.

In addition to synthesizing a beta-lactam product (e.g., amoxicillin or cephalexin) from a precursor, a PGA may also hydrolyze a beta-lactam precursor into an undesired by-product. A PGA's ability to synthesize a beta-lactam product from a precursor compared to its ability to hydrolyze an undesired by-product can be monitored by calculating a synthesis: hydrolysis ratio (S:H ratio). An S:H ratio greater than 1.0 indicates that a PGA uses more precursor for beta-lactam production than by-product production. An S:H ratio less than 1.0 indicates that a PGA uses more precursor for by-product production than beta-lactam production. In some embodiments, the S:H ratio of a PGA associated with the disclosure compared to the S:H ratio of a positive control PGA is improved by at least 1%, at least 5%, at least 10%, at least 15%, at least 20%, at least 25%, at least 30%, at least 40%, at least 50%, at least 60%, at least 70%, at least 80%, at least 90%, at least 100%, at least 150%, at least 200%, or at least 500%.

In some embodiments, a PGA that exhibits a higher S:H ratio than a control PGA comprises: an M at a residue corresponding to residue 181 in SEQ ID NO: 1; an F, I, L, N, or T at a residue corresponding to residue 182 in SEQ ID NO: 1; an F at a residue corresponding to residue 185 in SEQ ID NO: 1; an A at a residue corresponding to residue 326 in SEQ ID NO: 1; an A at a residue corresponding to residue 330 in SEQ ID NO: 1; an F at a residue corresponding to residue 362 in SEQ ID NO: 1; and/or a P at a residue corresponding to residue 482 in SEQ ID NO: 1.

In some embodiments, a PGA with improved amoxicillin S:H ratio properties compared to a positive control PGA comprises: M182N and F330A substitutions relative to the sequence of SEQ ID NO: 1; M182L, F330A, and T482P substitutions relative to the sequence of SEQ ID NO: 1; M182T, F330A, and L362F substitutions relative to the sequence of SEQ ID NO: 1; M182L, R185F, F330A, and T482P substitutions relative to the sequence of SEQ ID NO: 1; M182I and F330A substitutions relative to the sequence of SEQ ID NO: 1; R185F, N326A, F330A, and T482P substitutions relative to the sequence of SEQ ID NO: 1; T181M and F330A substitutions relative to the sequence of SEQ ID NO: 1; R185F, F330A, and T482P substitutions relative to the sequence of SEQ ID NO: 1; or T181M, M182F, F330A, and L362F substitutions relative to the sequence of SEQ ID NO: 1.

In some embodiments, a host cell capable of producing amoxicillin expresses a PGA that comprises F330A and W333I substitutions relative to the sequence of SEQ ID NO: 1. In some embodiments, a PGA with improved amoxicillin S:H ratio properties compared to a control PGA comprises F330A and W333I substitutions relative to the sequence of SEQ ID NO: 1.

In some embodiments, a PGA with improved cephalexin synthesis rate and S:H ratio properties compared to a positive control PGA comprises: T90V, A189V, F330A, W333I, D380I, and H498L substitutions relative to the sequence of SEQ ID NO: 1; T90V, A189V, F330A, D380V, H498L, and V563Q substitutions relative to the sequence of SEQ ID NO: 1; T90V, A189V, F330A, W333I, and D380V substitutions relative to the sequence of SEQ ID NO: 1; T90V, A189V, F330A, W333T, D380V, and H498S substitutions relative to the sequence of SEQ ID NO: 1; F330A and L770A substitutions relative to the sequence of SEQ ID NO: 1; T181M and F330A substitutions relative to the sequence of SEQ ID NO: 1; F330A and W333P substitutions relative to the sequence of SEQ ID NO: 1; an F330G substitutions relative to the sequence of SEQ ID NO: 1; F330A and W333I substitutions relative to the sequence of SEQ ID NO: 1; F330A and L770T substitutions relative to the sequence of SEQ ID NO: 1; F330A and W333Y substitutions relative to the sequence of SEQ ID NO: 1; or M182T, F330A, and L362F substitutions relative to the sequence of SEQ ID NO: 1.

In some embodiments, a host cell capable of producing cephalexin expresses a PGA that comprises M182I and F330A substitutions relative to the sequence of SEQ ID NO: 1. In some embodiments, a PGA with improved cephalexin synthesis properties and improved cephalexin S:H ratio properties compared to a control PGA comprises M182I and F330A substitutions relative to the sequence of SEQ ID NO: 1.

In some embodiments, a PGA comprises at least 1, at least 2, at least 3, at least 4, at least 5, at least 6, at least 7, at least 8, at least 9, at least 10, at least 11, at least 12, at least 13, at least, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 80, at least 90, or at least 100 amino acid substitutions, deletions, insertions, or additions relative to SEQ ID NO: 1, SEQ ID NO: 83, SEQ ID NO: 85, SEQ ID NO: 120, or a PGA enzyme otherwise described in this disclosure.

In some embodiments, a PGA associated with the disclosure may increase conversion of 6-APA and D-HPGM to D-amoxicillin by 1.1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, a PGA associated with the disclosure may increase conversion of 7-ADCA and D-PGM to cephalexin by 1.1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, the control is a PGA that comprises the sequence of SEQ ID NO: 1 or SEQ ID NO: 83.

In some embodiments, a PGA associated with the disclosure may exhibit at least 1.1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on 6-APA and D-HPGM relative to other available substrates. In some embodiments, a PGA associated with the disclosure may exhibit at least 1.1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on 7-ADCA and D-PGM relative to other available substrates.

Unless otherwise noted, the term “sequence identity” refers to the relatedness of the sequences of two polypeptides or polynucleotides when the sequences are aligned, and the term “percent identity” refers to the percentage of residues (amino acids or nucleotides) that are identical when two or more polypeptide or polynucleotide sequences are aligned. In some embodiments, sequence identity and/or percent identity is determined across the entire length of a sequence (e.g., PGA sequence). In some embodiments, sequence identity is determined over a region (e.g., a stretch of amino acids or nucleic acids, e.g., the sequence spanning an active site) of a sequence (e.g., PGA sequence). In some embodiments, sequence identity is determined over the length of an alpha subunit of a PGA and/or over the length of a beta subunit of a PGA.

Percent identity of polypeptide or polynucleotide sequences can be calculated by any of the methods known to one of ordinary skill in the art. For example, percent identity can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3. Where gaps exist between two sequences, Gapped BLAST® can be utilized, for example, as described in Altschul et al., Nucleic Acids Res. 25 (17): 3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

A second example of a local alignment technique is based on the Smith-Waterman algorithm (Smith, T. F. & Waterman, M. S. (1981) J. Mol. Biol. 147:195-197). An example of a global alignment technique is the Needleman-Wunsch algorithm (Needleman, S. B. & Wunsch, C. D. (1970) J. Mol. Biol. 48:443-453), which is based on dynamic programming. A further example of a global alignment technique is the Fast Optimal Global Sequence Alignment Algorithm (FOGSAA).

In some embodiments, the identity of two polypeptide sequences is determined by aligning the two amino acid sequences of the polypeptides, calculating the number of identical amino acids, and dividing by the length of one of the polypeptide sequences. In some embodiments, the identity of two polynucleotide sequences is determined by aligning the two nucleotide sequences of the polynucleotides, calculating the number of identical nucleotides and dividing by the length of one of the polynucleotide sequences.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) J. Mol. Biol. 147:195-197) or the Needleman-Wunsch algorithm (Needleman, S. B. & Wunsch, C. D. (1970) J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this disclosure, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using sequence alignment tools known in the art, such as, for example, Clustal Omega or BLAST®. For example, alpha and beta subunit residue positions in PGAs can be determined by aligning an alpha or beta subunit sequence (e.g., any one of SEQ ID NOs: 3-6, 8, 10, 11, 13, 18, 20, 21, 23-25, 27-32, or 34-39) with SEQ ID NO: 1 using an alignment algorithm described in this disclosure. One of ordinary skill in the art would recognize how to determine the residue in an alpha or beta subunit of a PGA that corresponds to a given residue within the sequence of SEQ ID NO: 1.

Variant sequences may be homologous sequences. As used in this disclosure, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between) and include but are not limited to paralogous, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant (e.g., PGA variant) comprises a domain that shares a secondary structure (e.g., alpha helix, beta sheet) with a reference polypeptide (e.g., a reference PGA). In some embodiments, a polypeptide variant (e.g., PGA variant) shares a tertiary structure with a reference polypeptide (e.g., a reference PGA). As a non-limiting example, a variant polypeptide (e.g., PGA variant) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by any method known to one of ordinary skill in the art. For example, mutations can be made by gene editing tools, PCR, site-directed mutagenesis (e.g., according to Kunkel, Proc. Nat. Acad. Sci. U.S.A. 82:488-492, 1985), chemical synthesis of a gene or polypeptide, or by insertions, such as insertion of a tag (e.g., a HIS tag or a GFP tag). Mutations can include, for example, substitutions, deletions, additions, insertions, fusions, and translocations, generated by any method known in the art.

In some embodiments, methods for producing variants include circular permutation (Yu and Lutz, Trends Biotechnol. 2011 January; 29 (1): 18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) compared to the linear sequence of the polypeptide before it was circularized and severed as determined by linear sequence alignment methods (e.g., Clustal Omega or BLAST). Topological analysis of the two polypeptides, however, may reveal that their tertiary structure is similar. Without being bound by a particular theory, a variant polypeptide created through circular permutation of a reference polypeptide and with a tertiary structure similar to the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29 (1): 18-25.

It should be appreciated that in a polypeptide that has undergone circular permutation, the linear amino acid sequence of the polypeptide would differ from a reference polypeptide that has not undergone circular permutation. However, one of ordinary skill in the art would be able to readily determine which residues in the polypeptide that has undergone circular permutation correspond to residues in the reference polypeptide that has not undergone circular permutation by, for example, aligning the sequences and detecting conserved motifs, and/or by comparing the structures or predicted structures of the polypeptides, e.g., by homology modeling. Variants described in this application include circularly permutated variants of sequences described in this application.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21 (7): 932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of PGAs disclosed in this application are encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins.

Putative functional variants may also be identified by searching for polypeptides with functionally annotated domains. Databases including Pfam (Sonnhammer et al., Proteins. 1997 July; 28 (3): 405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11; 10 (9): 2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and a mutant, such as a point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{cale} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63 (2): 337-346. Doi: 10.1016/j.molcel.2016.06.012.

In some embodiments, a polynucleotide sequence encoding a PGA comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 nucleotide positions corresponding to a reference sequence. In some embodiments, the polynucleotide sequence encoding the PGA comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of a coding sequence relative to a reference PGA coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the PGA coding sequence relative to the amino acid sequence of a reference PGA polypeptide.

In some embodiments, the one or more mutations in a polynucleotide sequence encoding a PGA sequence alters the amino acid sequence of the PGA polypeptide relative to the amino acid sequence of a reference PGA polypeptide. In some embodiments, the one or more mutations alters the amino acid sequence of the PGA polypeptide relative to the amino acid sequence of a reference PGA polypeptide and alters (enhances or reduces) an activity of the PGA polypeptide relative to the reference PGA polypeptide.

The activity (e.g., specific activity) of any of the polypeptides described in this disclosure (e.g., PGAs) may be measured using methods known in the art. As a non-limiting example, a polypeptide's activity may be determined by measuring its substrate specificity, product(s) produced, the concentration of product(s) produced, or any combination thereof. As used in this disclosure, “specific activity” of a polypeptide refers to the amount (e.g., concentration) of a particular product produced for a given amount (e.g., concentration) of the polypeptide per unit time.

Mutations in a polypeptide coding sequence may result in conservative amino acid substitutions. As used in this application, a “conservative amino acid substitution,” or “conservatively substituted amino acid” refers to an amino acid substitution that does not alter the relative charge or size characteristics or functional activity of the protein in which the amino acid substitution is made.

In some instances, an amino acid is characterized by its R group (see, e.g., Table 2). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group include lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Functionally equivalent variants of polypeptides may include conservative amino acid substitutions. Non-limiting examples of conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 2.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 2
Non-limiting examples of conservative amino acid substitutions.

| Original | | Conservative Amino |
|---|---|---|
| Residue | R Group Type | Acid Substitutions |
| Ala (A) | nonpolar aliphatic R group | Cys, Gly, Ser |
| Arg (R) | positively charged R group | His, Lys |
| Asn (N) | polar uncharged R group | Asp, Gln, Glu |
| Asp (D) | negatively charged R group | Asn, Gln, Glu |
| Cys (C) | polar uncharged R group | Ala, Ser |
| Gln (Q) | polar uncharged R group | Asn, Asp, Glu |
| Glu (E) | negatively charged R group | Asn, Asp, Gln |
| Gly (G) | nonpolar aliphatic R group | Ala, Ser |
| His (H) | positively charged R group | Arg, Tyr, Trp |
| Ile (I) | nonpolar aliphatic R group | Leu, Met, Val |
| Leu (L) | nonpolar aliphatic R group | Ile, Met, Val |
| Lys (K) | positively charged R group | Arg, His |
| Met (M) | nonpolar aliphatic R group | Ile, Leu, Phe, Val |
| Pro (P) | polar uncharged R group | |
| Phe (F) | nonpolar aromatic R group | Met, Trp, Tyr |
| Ser (S) | polar uncharged R group | Ala, Gly, Thr |
| Thr (T) | polar uncharged R group | Ala, Asn, Ser |
| Trp (W) | nonpolar aromatic R group | His, Phe, Tyr, Met |
| Tyr (Y) | nonpolar aromatic R group | His, Phe, Trp |
| Val (V) | nonpolar aliphatic R group | Ile, Leu, Met, Thr |

Amino acid substitutions in the amino acid sequence of a polypeptide to produce a polypeptide (e.g., PGA) variant having a desired property and/or activity can be made by alteration of the coding sequence of the polypeptide (e.g., PGA). Similarly, conservative amino acid substitutions in the amino acid sequence of a polypeptide to produce functionally equivalent variants of the polypeptide typically are made by alteration of the coding sequence of the polypeptide (e.g., PGA).

### Polynucleotides Encoding PGAs

Aspects of the present disclosure relate to recombinant enzymes, functional modifications and variants thereof, as well as uses relating thereto. For example, the enzymes and cells described in this application may be used to increase production of D-amoxicillin or cephalexin. The methods may comprise using a host cell comprising one or more enzymes disclosed in this application, a cell lysate, isolated enzymes, or any combination thereof. Methods comprising recombinant expression of polynucleotides encoding an enzyme disclosed in this application in a host cell are encompassed by the present disclosure. In vitro methods comprising reacting one or more PGAs in a reaction mixture disclosed in this application are also encompassed by the present disclosure.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system; or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is: situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13 (7): 563-567. A heterologous polynucleotide may comprise a wild-type sequence or a mutant sequence as compared with a reference polynucleotide sequence.

A polynucleotide encoding any one or more of the polypeptides (e.g., PGA) associated with the disclosure may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector). The vector may be a cloning vector, such as a plasmid, fosmid, phagemid, virus genome or artificial chromosome.

As used in this application, the terms “expression vector” or “expression construct” refer to a nucleic acid construct, generated recombinantly or synthetically, with a series of specified nucleic acid elements that permit transcription of a particular polynucleotide in a host cell (e.g., microbe). In some embodiments, a polynucleotide associated with the disclosure is inserted into an expression vector or expression construct such that it is operably joined to regulatory sequences and, in some embodiments, expressed as an RNA transcript. In some embodiments, the expression vector or expression construct contains one or more markers, such as a selectable marker, to identify cells transformed or transfected with the expression vector or expression construct. In some embodiments, a host cell has already been transformed with one or more vectors. In some embodiments, a host cell that has been transformed with one or more vectors is subsequently transformed with one or more vectors. In some embodiments, a host cell is transformed simultaneously with more than one vector. In some embodiments, a cell that has been transformed with a vector or an expression cassette incorporates all or part of the vector or expression cassette into its genome A polynucleotide encoding a polypeptide associated with the disclosure is “operably joined” or “operably linked” to a regulatory sequence when the polynucleotide and the regulatory sequence are covalently linked and the expression or transcription of the polynucleotide is under the influence or control of the regulatory sequence.

In some embodiments, the polynucleotide encoding any one or more of the polypeptides described in this application is under the control of regulatory sequences (e.g., enhancer sequences). In some embodiments, a polynucleotide (e.g., a polynucleotide comprising a gene) is expressed under the control of a promoter. In some embodiments, the promoter is a native promoter, corresponding to the promoter of the gene in its endogenous context. In other embodiments, the promoter is not the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include PmgrB, Ptrc2, PCI857, Pbad, Plac/ara, Plac/fnr, Ptac, Ptet, Pcmt, and Pm. In some embodiments, any promoter known in the art and suitable for a selected host cell can be used.

In some embodiments, the promoter is an inducible promoter. As used in this disclosure, an “inducible promoter” is a promoter controlled by the presence or absence of a molecule. This may be used, for example, to controllably induce the expression of an enzyme. In some embodiments, where an inducible promoter is linked to a PGA, the expression of PGA may be induced or not induced at certain times. Non-limiting examples of inducible promoters include chemically regulated promoters and physically regulated promoters. For chemically regulated promoters, the transcriptional activity can be regulated by one or more compounds, such as alcohol, an antibiotic such as tetracycline, a carbon source such as galactose, a steroid, a metal, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein ((TA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters known to one of ordinary skill in the art are also contemplated in this application.

In some embodiments, introduction of a polynucleotide, such as a polynucleotide encoding a polypeptide associated with the disclosure, into a host cell results in genomic integration of the polynucleotide. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a polynucleotide sequence, such as a polynucleotide sequence encoding any of the polypeptides described in this application, in its genome.

In some embodiments, the sequence of a polynucleotide (e.g., a polynucleotide comprising a gene) is codon-optimized. Codon optimization may increase expression of a gene by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

### Host Cells

Any of the polynucleotides or polypeptides of the disclosure may be expressed in a host cell. As used in this application, the term “host cell” refers to a cell that can be used to express a polynucleotide, such as a polynucleotide that encodes a PGA or a portion thereof, e.g., a polynucleotide which encodes a PGA comprising an optional signal peptide, an alpha subunit, an optional spacer, and a beta subunit; a polynucleotide encoding a PGA alpha subunit and a PGA beta subunit; or a first polynucleotide encoding a PGA alpha subunit and a second polynucleotide encoding a PGA beta subunit; etc.

Any suitable host cell may be used to produce any of the polypeptides, including PGAs and other polypeptides disclosed in this application, including eukaryotic cells or prokaryotic cells. Suitable host cells include, but are not limited to: yeast cells, bacterial cells, algal cells, plant cells, fungal cells, insect cells, and animal cells, including mammalian cells.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azotobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas. In some embodiments, the host cell is an E. coli cell.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure may also be suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure may also be suitable for use with a variety of plant cell types. The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart.

A vector or polynucleotide encoding any one or more of the polypeptides (e.g., PGA) described in this application may be introduced into a suitable host cell using any method known in the art. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cells. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place, involving a living organism or part of a living organism. Any type of bioreactor or fermenter known in the art may be compatible with aspects of the disclosure, including large or industrial scale bioreactors such as those with volumes in the range of liters or hundreds or thousands of liters or more.

In some embodiments, a bioreactor comprises a cell (e.g., a bacterial cell) or a cell culture (e.g., a bacterial cell culture), such as a cell or cell culture described in this application. In some embodiments, a bioreactor comprises a spore and/or a dormant cell type of an isolated microbe (e.g., a dormant cell in a dry state).

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., bacterial cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprise porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure such parameters are well known to one of ordinary skill in the relevant mechanical and electronic arts. Control systems to adjust the parameters in a bioreactor based on the inputs from a sensor are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated.

In some embodiments, the cells comprising a PGA of the present disclosure are adapted to consume 6-APA and D-HPGM in vivo. In some embodiments, the cells comprising a PGA of the present disclosure are adapted to consume 7-ADCA and D-PGM in vivo.

### Methods

In some aspects, the disclosure provides a method comprising culturing a host cell described in this application (e.g., a host cell comprising a heterologous polynucleotide encoding a PGA). Methods for culturing cells are described elsewhere in this application. In some embodiments, the disclosure provides a method of producing a β-Lactam from a β-Lactam precursor. In some embodiments, the disclosure provides a method of producing D-amoxicillin from 6-APA and D-HPGM, comprising culturing a host cell described in this application (e.g., a host cell comprising a heterologous polynucleotide encoding a PGA). In some embodiments, the disclosure provides a method of producing cephalexin from 7-ADCA and D-PGM, comprising culturing a host cell described in this application (e.g., a host cell comprising a heterologous polynucleotide encoding a PGA). Compositions, cells, enzymes, and methods described in this application are also applicable to industrial settings, including any application wherein there may be a need to reduce or eliminate bacterial presence.

Aspects of the present disclosure relate to a method of converting 6-APA and D-HPGM to D-amoxicillin, comprising contacting 6-APA and D-HPGM with a PGA wherein the PGA comprises a sequence that is at least 90% identical to SEQ ID NO: 1 or SEQ ID NO: 83.

Aspects of the present disclosure relate to a method of converting 7-ADCA and D-phenylglycine methyl ester D-PGM to cephalexin, comprising contacting 7-ADCA and D-PGM with a PGA wherein the PGA comprises a sequence that is at least 90% identical to SEQ ID NO: 1 or SEQ ID NO: 83. In some embodiments, the PGA is encoded by a polynucleotide comprising a sequence that is at least 90% identical to SEQ ID NO: 2 or SEQ ID NO: 84. In some embodiments, the PGA comprises one or more amino acid substitutions relative to the sequence of SEQ ID NO: 1 or SEQ ID NO: 83.

Aspects of the present disclosure relate to a method of converting 6-APA and D-HPGM to D-amoxicillin, comprising contacting 6-APA and D-HPGM with a PGA, wherein the PGA comprises an amino acid substitution at one or more of the following amino acid residues relative to the sequence of SEQ ID NO: 1: T90, T181, M182, R185, A189, N326, F330, W333, L362, D380, T482, H498, V563 and/or L770.

Aspects of the present disclosure relate to a method of converting 7-ADCA and D-PGM to cephalexin, comprising contacting 7-ADCA and D-PGM with a PGA, wherein the PGA comprises an amino acid substitution at one or more of the following amino acid residues relative to the sequence of SEQ ID NO: 1: T90, T181, M182, R185, A189, N326, F330, W333, L362, D380, T482, H498, V563 and/or L770.

In some embodiments, the PGA comprises one or more of the following amino acid substitutions relative to the sequence of SEQ ID NO: 1: T90V, T181M, M182N, M182L, M182T, M182I, M182F, R185F, A189V, N326A, F330A, F330G, W333Y, W333I, W333P, W333T, L362F, D380Q, D380I, D380V, T482P, H498S, H498L, V563Q, L770T and/or L770A.

In some embodiments, the PGA comprises the following amino acid substitutions relative to the sequence of SEQ ID NO: 1: (i) F330A and W333I; (ii) M182I and F330A; (iii) T90V, A189V, F330A, W333I and D380V; (iv) T90V, A189V, F330A, D380V, H498L and V563Q; (v) M182L, F330A and T482P; (vi) T90V, A189V, F330A, W333I, D380I and H498L; (vii) M182T, F330A and L362F; (viii) M182L, R185F, F330A and T482P; (ix) F330G; (x) F330A and W333Y; (xi) R185F, N326A, F330A and T482P; (xii) F330A and W333P; (xiii) F330A and L770T; (xiv) T181M and F330A; (xv) F330A and D380Q; (xvi) M182N and F330A; (xvii) R185F, F330A and T482P; (xviii) F330A and L770A; (xix) T90V, A189V, F330A, W333T, D380V and H498S; or (xx) T181M, M182F, F330A and L362F. In some embodiments, the PGA comprises two or more of said amino acid substitutions selected from (i) to (xx).

In some embodiments, the present disclosure relates to a method of converting 6-APA and D-HPGM to D-amoxicillin, comprising contacting the 6-APA and the D-HPGM with a PGA, wherein the PGA comprises one or more amino acid substitutions relative to the sequence of SEQ ID NO: 1, selected from a group comprising: (i) F330A and W333I; (ii) M182I and F330A; (iii) T90V, A189V, F330A, W333I and D380V; (iv) T90V, A189V, F330A, D380V, H498L and V563Q; (v) M182L, F330A and T482P; (vi) T90V, A189V, F330A, W333I, D380I and H498L; (vii) M182T, F330A and L362F; (viii) M182L, R185F, F330A and T482P; (ix) F330G; (x) F330A and W333Y; (xi) R185F, N326A, F330A and T482P; (xii) F330A and W333P; (xiii) F330A and L770T; (xiv) T181M and F330A; (xv) F330A and D380Q; (xvi) M182N and F330A; (xvii) R185F, F330A and T482P; (xviii) F330A and L770A; (xix) T90V, A189V, F330A, W333T, D380V and H498S; or (xx) T181M, M182F, F330A and L362F.

In some embodiments, the present disclosure relates to a method of converting 6-APA and D-HPGM to D-amoxicillin, comprising contacting the 6-APA and the D-HPGM with a PGA, wherein the PGA comprises one or more amino acid substitutions relative to the sequence of SEQ ID NO: 1, selected from a group comprising: (i) F330A and W333Y; (ii) T90V, A189V, F330A, W333I and D380V; (iii) T90V, A189V, F330A, D380V, H498L and V563Q; (iv) T90V, A189V, F330A, W333I, D380I and H498L; (v) F330G; (vi) F330A and W333P; (vii) F330A and L770T; (viii) F330A and D380Q; (ix) F330A and W333I; (x) F330A and L770A; and (xi) T90V, A189V, F330A, W333T, D380V and H498S. In some embodiments, the PGA comprises two or more of said amino acid substitutions selected from (i) to (xi).

In some embodiments, the present disclosure relates to a method of converting 7-ADCA and D-PGM to cephalexin, comprising contacting 7-ADCA and D-PGM with a PGA, wherein the PGA comprises one or more amino acid substitutions relative to the sequence of SEQ ID NO: 1, selected from a group comprising: (i) F330A and W333I; (ii) M182I and F330A; (iii) T90V, A189V, F330A, W333I and D380V; (iv) T90V, A189V, F330A, D380V, H498L and V563Q; (v) M182L, F330A and T482P; (vi) T90V, A189V, F330A, W333I, D380I and H498L; (vii) M182T, F330A and L362F; (viii) M182L, R185F, F330A and T482P; (ix) F330G; (x) F330A and W333Y; (xi) R185F, N326A, F330A and T482P; (xii) F330A and W333P; (xiii) F330A and L770T; (xiv) T181M and F330A; (Xv) F330A and D380Q; (xvi) M182N and F330A; (xvii) R185F, F330A and T482P; (xviii) F330A and L770A; (xix) T90V, A189V, F330A, W333T, D380V and H498S; or (xx) T181M, M182F, F330A and L362F.

In some embodiments, the present disclosure relates to a method of converting 7-ADCA and D-PGM to cephalexin, comprising contacting 7-ADCA and D-PGM with a PGA, wherein the PGA comprises one or more amino acid substitutions relative to the sequence of SEQ ID NO: 1, selected from a group comprising: (i) F330A and W333Y; (ii) T90V, A189V, F330A, W333I and D380V; (iii) T90V, A189V, F330A, D380V, H498L and V563Q; (iv) T90V, A189V, F330A, W333I, D380I and H498L; (v) F330G; (vi) F330A and W333P; (vii) F330A and L770T; (viii) F330A and D380Q; (ix) F330A and W333I; (x) F330A and L770A; and (xi) T90V, A189V, F330A, W333T, D380V and H498S.

In some embodiments, the alpha subunit of the PGA comprises one or more of the following amino acid substitutions relative to the sequence of SEQ ID NO: 1: T90V, T181M, M182N, M182L, M182T, M182I, M182F, R185F, and/or A189V.

In some embodiments, the beta subunit of the PGA comprises one or more of the following amino acid substitutions relative to the sequence of SEQ ID NO: 1: N326A, F330A, F330G, W333Y, W333I, W333P, W333T, L362F, D380Q, D380I, D380V, T482P, H498S, H498L, V563Q, L770T and/or L770A.

In some embodiments, the beta subunit of the PGA comprises the mutation F330A and one or more of the following amino acid substitutions relative to the sequence of SEQ ID NO: 1: N326A, W333Y, W333I, W333P, W333T, L362F, D380Q, D380I, D380V, T482P, H498S, H498L, V563Q, L770T and/or L770A.

In some embodiments, the alpha subunit of the PGA comprises one or more of the following amino acid substitutions relative to the sequence of SEQ ID NO: 1: T90V, T181M, M182N, M182L, M182T, M182I, M182F, R185F, and/or A189V; and the beta subunit of the PGA comprises one or more of the following amino acid substitutions relative to the sequence of SEQ ID NO: 1: N326A, F330A, F330G, W333Y, W333I, W333P, W333T, L362F, D380Q, D380I, D380V, T482P, H498S, H498L, V563Q, L770T and/or L770A.

In some embodiments, the alpha subunit of the PGA comprises one or more of the following amino acid substitutions relative to the sequence of SEQ ID NO: 1: T90V, T181M, M182N, M182L, M182T, M182I, M182F, R185F, and/or A189V; and the beta subunit of the PGA comprises the mutation F330A and one or more of the following amino acid substitutions relative to the sequence of SEQ ID NO: 1: N326A, W333Y, W333I, W333P, W333T, L362F, D380Q, D380I, D380V, T482P, H498S, H498L, V563Q, L770T and/or L770A.

In some embodiments, a PGA comprises an alpha and a beta subunit, wherein the PGA comprises the following amino acid substitutions relative to the sequence of SEQ ID NO: 1: (a) F330A and (b) any one or more of: (i) W333I; (ii) M182I; (iii) T90V, A189V, W333I and D380V; (iv) T90V, A189V, D380V, H498L and V563Q; (v) M182L, T482P; (vi) T90V, A189V, W333I, D380I and H498L; (vii) M182T, L362F; (viii) M182L, R185F, T482P; (ix) W333Y; (x) R185F, N326A and T482P; (xi) W333P; (xii) L770T; (xiii) T181M; (xiv) D380Q; (xv) M182N; (xvi) R185F and T482P; (xvii) L770A; (xviii) T90V, A189V, W333T, D380V and H498S; or (xix) T181M, M182F and L362F.

Aspects of the present disclosure relate to a method of converting 6-APA and D-HPGM to D-amoxicillin, comprising contacting 6-APA and D-HPGM with a PGA, wherein the PGA comprises an amino acid substitution at one or more of the following amino acid residues relative to the sequence of SEQ ID NO: 1: T90, A189, F330, W333, D380, H498, V563 and/or L770.

Aspects of the present disclosure relate to a method of converting 7-ADCA and D-PGM to cephalexin, comprising contacting 7-ADCA and D-PGM with a PGA, wherein the PGA comprises an amino acid substitution at one or more of the following amino acid residues relative to the sequence of SEQ ID NO: 1: T90, A189, F330, W333, D380, H498, V563 and/or L770.

In some embodiments, the PGA comprises one or more of the following amino acid substitutions relative to the sequence of SEQ ID NO: 1: T90V, A189V, F330A, F330G, W333I, W333Y, W333P, W333T, D380V, D380Q, D380I, H498S, H498L, V563Q, L770T and/or L770A. In some embodiments, the PGA comprises the following amino acid substitutions relative to the sequence of SEQ ID NO: 1: (i) F330A and W333Y; (ii) T90V, A189V, F330A, W333I and D380V; (iii) T90V, A189V, F330A, D380V, H498L and V563Q; (iv) T90V, A189V, F330A, W333I, D380I and H498L; (v) F330G; (vi) F330A and W333P; (vii) F330A and L770T; (viii) F330A and D380Q; (ix) F330A and W333I; (x) F330A and L770A; or (xi) T90V, A189V, F330A, W333T, D380V and H498S.

Aspects of the present disclosure relate to a method of converting 6-APA and D-HPGM to D-amoxicillin, comprising contacting 6-APA and D-HPGM with a PGA, wherein the PGA comprises an amino acid substitution at one or more of the following amino acid residues relative to the sequence of SEQ ID NO: 1: T90, T181, A189, N326, W333, L362, D380, H498 and/or V563.

Aspects of the present disclosure relate to a method of converting 7-ADCA and D-PGM to cephalexin, comprising contacting 7-ADCA and D-PGM with a PGA, wherein the PGA comprises an amino acid substitution at one or more of the following amino acid residues relative to SEQ ID NO: 1: T90, T181, A189, N326, W333, L362, D380, H498 and/or V563.

In some embodiments, the PGA comprises one or more of the following amino acid substitutions relative to the sequence of SEQ ID NO: 1: T90V, T181M, A189V, N326A, W333Y, W333I, W333P, W333T, L362F, D380Q, D380I, D380V, H498S, H498L and V563Q. In some embodiments, the PGA comprises a sequence that is at least 90% identical to SEQ ID NO: 1 or SEQ ID NO: 83.

Aspects of the present disclosure relate to a method of converting a β-lactam precursor to a β-lactam, comprising contacting the β-lactam precursor with a PGA associated with the disclosure. In some embodiments, the β-lactam is D-amoxicillin. In some embodiments, the β-lactam is cephalexin. In some embodiments, the β-lactam precursor is one or more of 6-APA, D-HPGM, 7-ADCA, and/or D-PGM. In some embodiments, a precursor is produced via any process known in the art, including (but not limited to: enzymatically, e.g., by a different enzyme; see, for example: IN202121011569A).

In some embodiments, a PGA described in the present disclosure is recovered or purified from a host cell. In some embodiments, a PGA is immobilized. In some embodiments, a PGA is immobilized after recovery from a host cell. In some embodiments, an immobilized PGA is enzymatically active. In some embodiments, an enzymatic reaction occurs using an immobilized PGA. In some embodiments, a PGA is immobilized on a solid surface. In some embodiments, a PGA is immobilized on a water-insoluble material. In some embodiments, a PGA is immobilized on a carrier. In some embodiments, a PGA is immobilized on a carrier consisting of a gelling agent and a polymer containing free amino groups. In some embodiments, a PGA is immobilized according to the methods described in CN101381718A; CN101381719A; CN103451259A; CN104120120A; CN105087533A; CN105274082A; EP0222462B1; EP0297912B1; JP2001527381A; KR101735126B1; KR20010100600A; U.S. Pat. Nos. 6,060,268; 6,060,268; 6,214,609; 8,753,856; WO 199704086A1; WO2001029202A1; WO2021140526A1; or WO2021140526A1, the contents of each of which are hereby incorporated by reference in their entireties.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

In order that the invention described in the present application may be more fully understood, the following examples are set forth. The examples described in this application are offered to illustrate the systems and methods provided in this disclosure and are not to be construed in any way as limiting their scope.

### Example 1. Functional Expression of Variant PGAs and Screening for Activity in Amoxicillin Synthesis

As outlined in FIG. 1, during D-amoxicillin production, 6-APA and D-HPGM are transformed enzymatically to D-amoxicillin by a PGA. The PGA can synthesize the desired product D-amoxicillin or it can hydrolyze the D-HPGM to produce the undesired by-product D-HPG. The ratio of the amount of desired “synthesis” product (D-amoxicillin) to the amount of “hydrolysis” by-product (D-HPG) is known as the synthesis: hydrolysis ratio (S:H ratio). The hydrolysis of D-amoxicillin product to 6-APA and D-HPG by PGA is an additional source of D-HPG by-product. Optimizing the S:H ratio to increase the amount of D-amoxicillin produced and reduce the amount of D-HPG produced is beneficial in the commercial production of D-amoxicillin. This Example relates to the engineering of a PGA from Achromobacter sp. CCM 4824 for improving the rate of D-amoxicillin formation as well as improving the S:H ratio.

To identify PGA variants with improved rates of D-amoxicillin formation and S:H ratios, the PGA from Achromobacter sp. CCM 4824 (SEQ ID NO: 1) was used as a template sequence to generate a library of approximately 1000 variants using a combination of sequence- and structure-based methods, including detailed analysis of various structural aspects of the alpha and beta subunits and various portions and components thereof. The signal sequence within SEQ ID NO: 1 was also removed and replaced with a different signal sequence.

Among the approximately 1000 variants tested in the primary screen, about 25% showed no enzymatic activity, as indicated by a lack of measurable D-amoxicillin or D-HPG in the samples, suggesting that certain mutations can result in a non-functional enzyme. Approximately 90% of the 1000 tested variants demonstrated performance which was less than the positive control, or not significantly improved relative to the positive control, and were not selected for additional screening in the secondary screens.

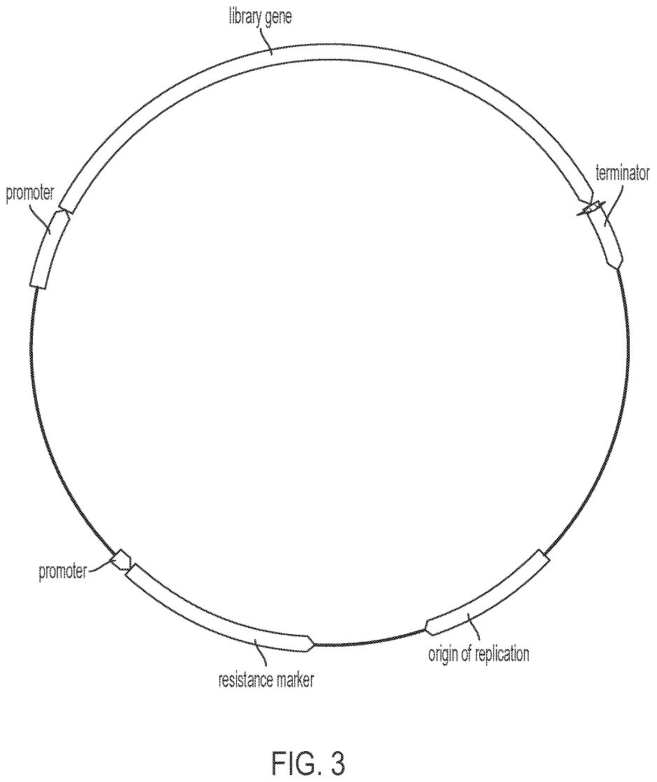

Protein sequences were encoded in nucleotide sequences using E. coli codon usage and were synthesized in the replicative E. coli expression vector shown in FIG. 3. Each variant enzyme expression construct was transformed into an expression strain derived from E. coli Rv311. Transformants were selected based on ability to grow on media containing 50 μg/mL kanamycin as a selection marker. Strain t1134458, expressing a construct encoding a fluorescent protein, was included in the library as a negative control for enzyme activity. Strain t1134457, expressing a construct encoding the PGA from Achromobacter sp. CCM 4824 with the same signal sequence as the library of PGA variants, and also including a F330A amino acid substitution relative to the sequence of SEQ ID NO: 1, was included in the library as a positive control. PGA variants with rates of D-amoxicillin formation above that of the positive control PGA expressed in strain t1134457 and/or with S:H ratios higher than that of the positive control PGA expressed in strain t1134457 identified in the screen were considered variant enzymes with improved activity.

The library of PGA variants was first assayed for activity in a primary screen for D-amoxicillin and D-HPG synthesis. E. coli transformants expressing the PGA variants were tested for D-amoxicillin and D-HPG production by growing clonal expression cultures with 3 biological replicates, followed by preparation of cell lysates, and feeding of the substrates, 6-APA and D-HPGM, under appropriate reaction conditions in vitro as described below. Mass spectrometric analysis of D-amoxicillin and D-HPG production was performed at 3 different timepoints as described below. A subset of the PGA variants in the library exhibited improved D-amoxicillin formation rate and/or improved S:H ratio compared to the PGA expressed by positive control strain t1134457.

### D-Amoxicillin Synthesis Assay—Primary Screen

300 nL/well of thawed glycerol stocks of PGA variant E. coli transformants were dispensed into 30 μL/well of Teknova ZY media containing 6.70 g/L sodium phosphate dibasic, 3.4 g/L potassium phosphate monobasic, 2.68 g/L ammonium chloride, 0.71 g/L sodium sulfate, 0.5% (w/v) glycerol, 0.05% (w/v) glucose, 0.05% (w/v) arabinose, 0.2× Trace minerals Teknova T1001, 2 mM MgSO4, and 200 μg/mL kanamycin in 384-well microtiter plates and scaled with AeraSeals. Samples were incubated at 20° C. for 36-40 hours, diluted with 60 μL of TBS, and centrifuged for 10 minutes at 4° C. and 4000×g. The supernatant was aspirated and cell pellets were frozen at −80° C.

Pellets were thawed by shaking for 20 minutes at 30° C., and then lysed by the addition of 45 μL of lysis buffer containing 40% (v/v) Bugbuster, 0.01% (v/v) Sigma rLysozyme, and 0.01% (v/v) Pierce Universal Nuclease. Lysate plates were sealed, shaken for 20 m at 1,000 rpm and 30° C. in an Infors Multitron HT incubator, and centrifuged for 10 s at 500 RCF. In parallel, 384 well microtiter plates were prepared containing reaction buffer and analytical standards. Samples corresponding to enzyme variants being tested contained 54 μL of reaction buffer with 0.89 mM 6-aminopenicillanic acid, 2.22 mM D-HPGM, and 22.2 mM MOPS pH 6. Analytical standard samples contained 22.2 mM MOPS pH 6 and varying concentrations of amoxicillin and hydroxyphenylglycine, ranging from zero to 0.889 mM (amoxicillin) and from zero to 2.22 mM (hydroxyphenylglycine). Reactions were initiated by stamping 6 μL of lysate into the reaction plates. Samples were incubated at room temperature, and at 2 hours, 4 hours, and 16 to 20 hours after the reaction was initiated 6.1 μL samples were withdrawn and added to 115 μL of quench buffer in a 384 deep well plate. Quench buffer was comprised of 0.23% (v/v) formic acid, 0.42% ammonium hydroxide, 21.0 μM of isotopically labeled hydroxyphenylglycine, 10.6 μM of isotopically labeled amoxicillin, and 0.5 mM phenylmethylsulfonyl fluoride. Samples were further quenched by addition of 1 μL of 100 mM phenylmethylsulfonyl fluoride in ethanol. Quenched samples were centrifuged for 10 minutes at 4,000, 50 μL of supernatant was removed into an Echo compatible microplate, and samples were stored at −80° C. until analysis by Echo-MS.

### Example 2. Secondary Screening of the PGA Variants for D-Amoxicillin Synthesis

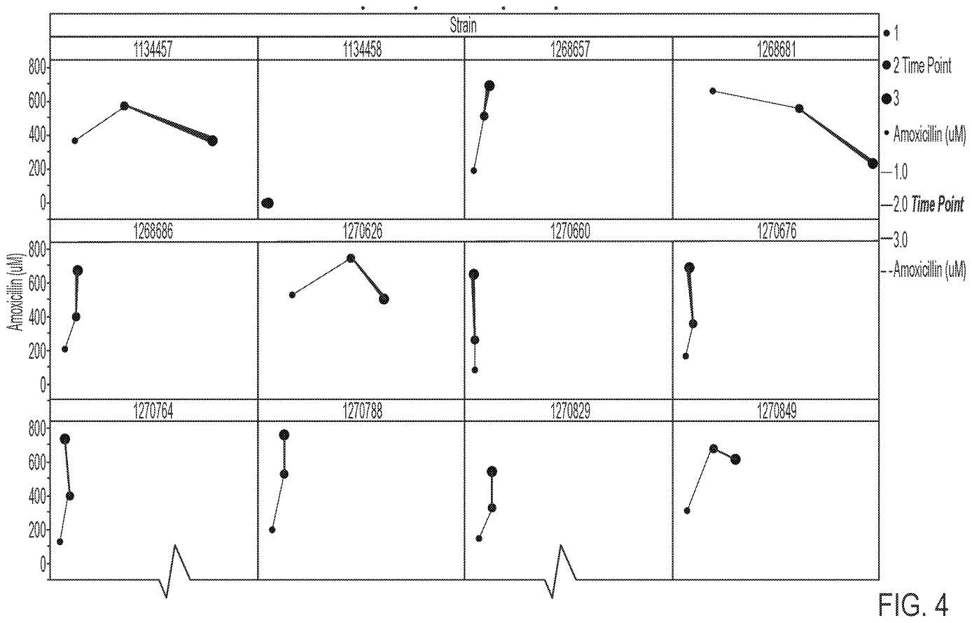

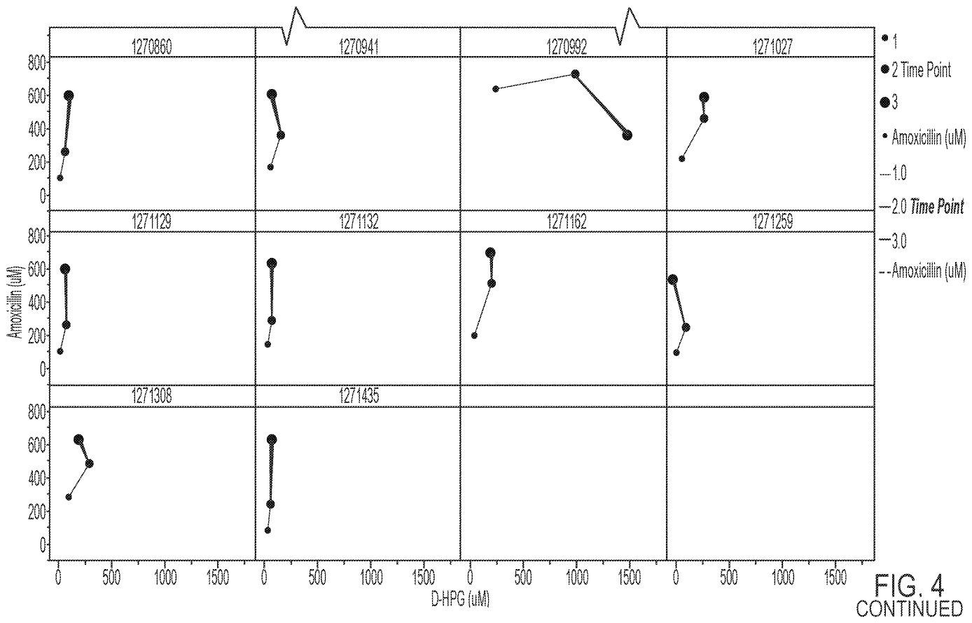

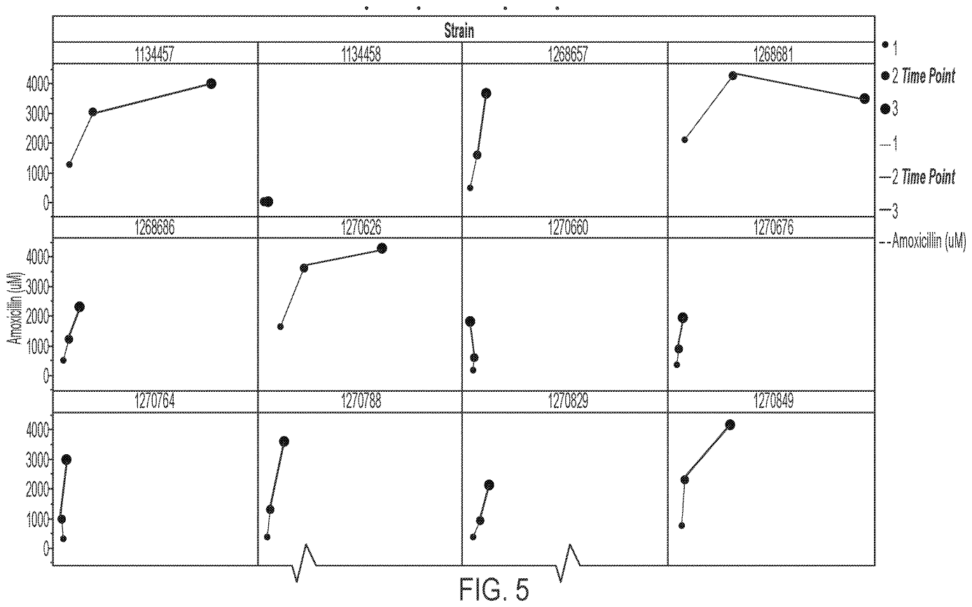

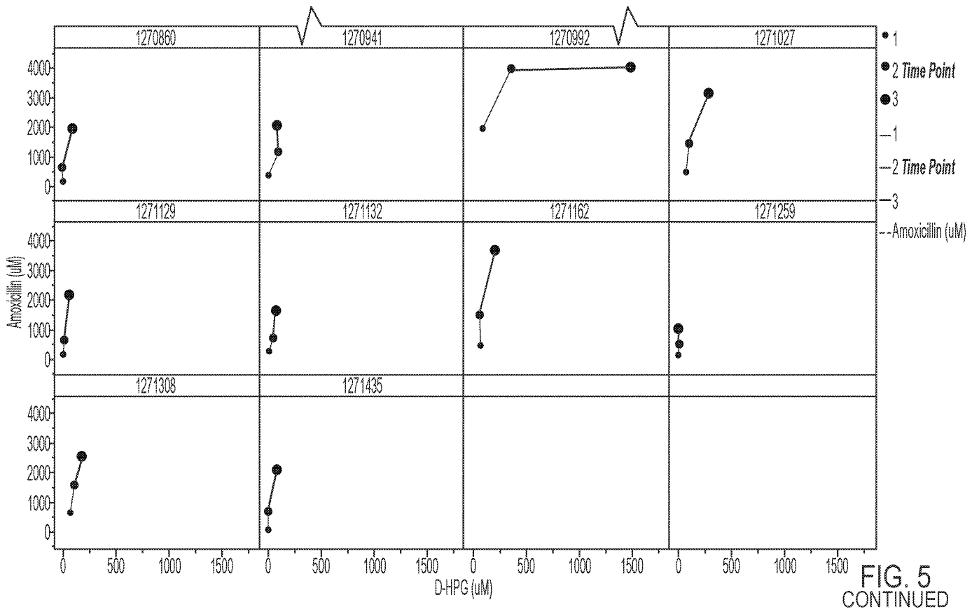

To confirm the improved activity of the subset of PGA variants identified in the primary screen described in Example 1, approximately ~10% of the PGA variant library described in Example 1, including the subset of hits identified in the primary screen, was screened in a secondary screen. The secondary screen included reaction conditions identical to the primary screen (“Condition 1”), as well as an additional set of in vitro reaction conditions (“Condition 2”) as described below and shown in FIGS. 4 and 5, respectively.

In the secondary screen, 20 PGA variants were found to have improved D-amoxicillin formation rate and/or improved S:H ratio compared to the positive control PGA expressed by strain t1134457.

Results from the secondary screen, including the D-amoxicillin concentration (μM) and D-HPG concentration (μM) at each of the 3 timepoints, the calculated S:H ratio at each of the 3 timepoints, and the initial D-amoxicillin formation rate (Condition 2 only) are summarized in Table 3 (Condition 1) and Table 4 (Condition 2).

TABLE 3
D-amoxicillin synthesis and S:H ratio exhibited by 20 PGA variants
under Condition 1 (values represent average of 3 replicates)

| | | | Average | Average | |
|---|---|---|---|---|---|
| | Time | | Amoxicillin | D-HPG | S:H |
| Strain | Point | Sample Type | (μM) | (μM) | ratio |
| t1134457 | 1 | positive control | 366.14 | 148.62 | 2.46 |
| t1134457 | 2 | positive control | 565.02 | 603.27 | 0.94 |
| t1134457 | 3 | positive control | 365.70 | 1,429.88 | 0.26 |
| t1134458 | 1 | negative control | −5.55 | 6.27 | −0.89 |
| t1134458 | 2 | negative control | −6.23 | −9.07 | 0.69 |
| t1134458 | 3 | negative control | −7.61 | 17.74 | −0.43 |
| t1268657 | 1 | library | 186.57 | 1.71 | 109.11 |
| t1268657 | 2 | library | 519.90 | 92.04 | 5.65 |
| t1268657 | 3 | library | 691.05 | 144.54 | 4.78 |
| t1268681 | 1 | library | 660.43 | 291.82 | 2.26 |
| t1268681 | 2 | library | 556.80 | 1,092.87 | 0.51 |
| t1268681 | 3 | library | 220.73 | 1,782.75 | 0.12 |
| t1268686 | 1 | library | 206.82 | 60.96 | 3.39 |
| t1268686 | 2 | library | 390.61 | 171.67 | 2.28 |
| t1268686 | 3 | library | 666.18 | 177.38 | 3.76 |
| t1270626 | 1 | library | 511.32 | 242.02 | 2.11 |
| t1270626 | 2 | library | 733.07 | 787.89 | 0.93 |
| t1270626 | 3 | library | 493.20 | 1,094.43 | 0.45 |
| t1270660 | 1 | library | 79.74 | 10.08 | 7.91 |
| t1270660 | 2 | library | 250.51 | 16.40 | 15.28 |
| t1270660 | 3 | library | 632.60 | −12.39 | −51.06 |
| t1270676 | 1 | library | 158.74 | 43.84 | 3.62 |
| t1270676 | 2 | library | 347.40 | 103.68 | 3.35 |
| t1270676 | 3 | library | 686.43 | 75.58 | 9.08 |
| t1270764 | 1 | library | 125.82 | 17.75 | 7.09 |
| t1270764 | 2 | library | 398.44 | 90.79 | 4.39 |
| t1270764 | 3 | library | 745.24 | 55.29 | 13.48 |
| t1270788 | 1 | library | 193.90 | 48.15 | 4.03 |
| t1270788 | 2 | library | 523.68 | 160.54 | 3.26 |
| t1270788 | 3 | library | 749.68 | 169.10 | 4.43 |
| t1270829 | 1 | library | 154.86 | 47.11 | 3.29 |
| t1270829 | 2 | library | 314.92 | 159.56 | 1.97 |
| 11270829 | 3 | library | 547.02 | 171.30 | 3.19 |
| t1270849 | 1 | library | 307.23 | 50.77 | 6.05 |
| t1270849 | 2 | library | 676.65 | 286.12 | 2.36 |
| t1270849 | 3 | library | 616.59 | 492.57 | 1.25 |
| t1270860 | 1 | library | 88.66 | 14.10 | 6.29 |
| t1270860 | 2 | library | 260.94 | 68.69 | 3.80 |
| t1270860 | 3 | library | 569.05 | 96.85 | 5.88 |
| t1270941 | 1 | library | 161.19 | 56.47 | 2.85 |
| t1270941 | 2 | library | 349.39 | 154.23 | 2.27 |
| t1270941 | 3 | library | 598.17 | 73.18 | 8.17 |
| t1270992 | 1 | library | 621.39 | 224.30 | 2.77 |
| t1270992 | 2 | library | 719.03 | 971.51 | 0.74 |
| t1270992 | 3 | library | 356.92 | 1,464.70 | 0.24 |
| t1271027 | 1 | library | 214.25 | 67.31 | 3.18 |
| t1271027 | 2 | library | 455.67 | 264.29 | 1.72 |
| t1271027 | 3 | library | 584.50 | 259.23 | 2.25 |
| t1271129 | 1 | library | 101.95 | 18.74 | 5.44 |
| t1271129 | 2 | library | 270.65 | 73.23 | 3.70 |
| t1271129 | 3 | library | 595.70 | 65.12 | 9.15 |
| t1271132 | 1 | library | 143.05 | 38.51 | 3.71 |
| t1271132 | 2 | library | 293.63 | 71.19 | 4.12 |
| t1271132 | 3 | library | 634.82 | 65.15 | 9.74 |
| t1271162 | 1 | library | 195.54 | 36.69 | 5.33 |
| t1271162 | 2 | library | 514.67 | 201.12 | 2.56 |
| t1271162 | 3 | library | 694.30 | 186.88 | 3.72 |
| t1271259 | 1 | library | 99.59 | 5.88 | 16.94 |
| t1271259 | 2 | library | 237.09 | 82.56 | 2.87 |
| t1271259 | 3 | library | 532.17 | −16.92 | −31.45 |
| t1271308 | 1 | library | 283.04 | 93.21 | 3.04 |
| t1271308 | 2 | library | 483.74 | 284.69 | 1.70 |
| t1271308 | 3 | library | 624.73 | 193.19 | 3.23 |
| 11271435 | 1 | library | 78.01 | 22.25 | 3.51 |
| t1271435 | 2 | library | 236.12 | 50.29 | 4.70 |
| t1271435 | 3 | library | 620.57 | 73.12 | 8.49 |

TABLE 4
D-amoxicillin synthesis and S:H ratio exhibited by 20 PGA variants
under Condition 2 (values represent average of 3 replicates)

| | Time | | Amoxicillin formation | Amoxicillin | D-HPG | |
|---|---|---|---|---|---|---|
| Strain | Point | Sample Type | rate (μM/h) | (μM) | (μM) | S:H ratio |
| t1134457 | 1 | positive | 635.15 | 1,270.31 | 90.13 | 14.09 |
| | | control | | | | |
| t1134457 | 2 | positive | 635.15 | 3,013.30 | 302.29 | 9.97 |
| | | control | | | | |
| t1134457 | 3 | positive | 635.15 | 3,942.32 | 1,412.00 | 2.79 |
| | | control | | | | |
| t1134458 | 1 | negative | 4.15 | 8.30 | 1.61 | 5.16 |
| | | control | | | | |
| t1134458 | 2 | negative | 4.15 | −5.31 | −4.03 | 1.32 |
| | | control | | | | |
| t1134458 | 3 | negative | 4.15 | 19.28 | 17.52 | 1.10 |
| | | control | | | | |
| t1268657 | 1 | library | 235.67 | 471.33 | −7.29 | −64.65 |
| t1268657 | 2 | library | 235.67 | 1,607.73 | 51.48 | 31.23 |
| t1268657 | 3 | library | 235.67 | 3,637.84 | 142.73 | 25.49 |
| t1268681 | 1 | library | 1,045.51 | 2,091.02 | 85.83 | 24.36 |
| t1268681 | 2 | library | 1,045.51 | 4,246.36 | 530.48 | 8.00 |
| t1268681 | 3 | library | 1,045.51 | 3,475.48 | 1,760.46 | 1.97 |
| t1268686 | 1 | library | 275.41 | 550.82 | 20.11 | 27.39 |
| t1268686 | 2 | library | 275.41 | 1,234.03 | 79.48 | 15.53 |
| t1268686 | 3 | library | 275.41 | 2,304.83 | 175.16 | 13.16 |
| t1270626 | 1 | library | 818.12 | 1,636.24 | 138.51 | 11.81 |
| t1270626 | 2 | library | 818.12 | 3,645.29 | 357.28 | 10.20 |
| t1270626 | 3 | library | 818.12 | 4,269.79 | 1,080.75 | 3.95 |
| t1270660 | 1 | library | 84.45 | 168.90 | 13.21 | 12.79 |
| t1270660 | 2 | library | 84.45 | 585.87 | 31.66 | 18.51 |
| t1270660 | 3 | library | 84.45 | 1,797.14 | −12.24 | −146.83 |
| t1270676 | 1 | library | 195.49 | 390.98 | 5.08 | 76.96 |
| t1270676 | 2 | library | 195.49 | 883.53 | 26.29 | 33.61 |
| t1270676 | 3 | library | 195.49 | 1,956.23 | 74.64 | 26.21 |
| t1270764 | 1 | library | 157.48 | 314.96 | 32.12 | 9.81 |
| t1270764 | 2 | library | 157.48 | 1,012.50 | 12.69 | 79.79 |
| t1270764 | 3 | library | 157.48 | 2,969.15 | 54.60 | 54.38 |
| t1270788 | 1 | library | 203.21 | 406.42 | 20.31 | 20.01 |
| t1270788 | 2 | library | 203.21 | 1,304.27 | 38.92 | 33.51 |
| t1270788 | 3 | library | 203.21 | 3,558.95 | 166.99 | 21.31 |
| t1270829 | 1 | library | 202.40 | 404.80 | 14.86 | 27.24 |
| t1270829 | 2 | library | 202.40 | 1,008.09 | 86.83 | 11.61 |
| t1270829 | 3 | library | 202.40 | 2,118.77 | 169.16 | 12.53 |
| t1270849 | 1 | library | 389.03 | 778.05 | 53.05 | 14.67 |
| t1270849 | 2 | library | 389.03 | 2,335.92 | 93.18 | 25.07 |
| t1270849 | 3 | library | 389.03 | 4,193.99 | 486.41 | 8.62 |
| t1270860 | 1 | library | 108.42 | 216.83 | 6.65 | 32.61 |
| t1270860 | 2 | library | 108.42 | 679.43 | −9.65 | −70.41 |
| t1270860 | 3 | library | 108.42 | 1,979.08 | 95.64 | 20.69 |
| t1270941 | 1 | library | 220.35 | 440.70 | −1.17 | −376.67 |
| t1270941 | 2 | library | 220.35 | 1,139.92 | 76.50 | 14.90 |
| t1270941 | 3 | library | 220.35 | 2,044.17 | 72.26 | 28.29 |
| t1270992 | 1 | library | 989.38 | 1,978.76 | 84.06 | 23.54 |
| t1270992 | 2 | library | 989.38 | 3,918.50 | 341.68 | 11.47 |
| t1270992 | 3 | library | 989.38 | 3,991.66 | 1,446.38 | 2.76 |
| t1271027 | 1 | library | 245.32 | 490.65 | 63.03 | 7.78 |
| t1271027 | 2 | library | 245.32 | 1,478.44 | 90.08 | 16.41 |
| t1271027 | 3 | library | 245.32 | 3,102.06 | 255.98 | 12.12 |
| t1271129 | 1 | library | 116.68 | 233.36 | −2.08 | −112.19 |
| t1271129 | 2 | library | 116.68 | 715.17 | 19.42 | 36.83 |
| t1271129 | 3 | library | 116.68 | 2,202.73 | 64.30 | 34.26 |
| t1271132 | 1 | library | 177.61 | 355.22 | 8.97 | 39.60 |
| t1271132 | 2 | library | 177.61 | 785.08 | 33.60 | 23.37 |
| t1271132 | 3 | library | 177.61 | 1,657.59 | 64.33 | 25.77 |
| t1271162 | 1 | library | 245.50 | 491.00 | 45.75 | 10.73 |
| t1271162 | 2 | library | 245.50 | 1,558.56 | 51.64 | 30.18 |
| t1271162 | 3 | library | 245.50 | 3,734.82 | 184.54 | 20.24 |
| t1271259 | 1 | library | 139.92 | 279.84 | −16.43 | −17.03 |
| t1271259 | 2 | library | 139.92 | 570.26 | −2.90 | −196.64 |
| t1271259 | 3 | library | 139.92 | 1,066.66 | −16.71 | −63.83 |
| t1271308 | 1 | library | 403.86 | 807.73 | 67.84 | 11.91 |
| t1271308 | 2 | library | 403.86 | 1,644.83 | 111.15 | 14.80 |
| t1271308 | 3 | library | 403.86 | 2,659.12 | 190.78 | 13.94 |
| t1271435 | 1 | library | 127.37 | 254.73 | −7.04 | −36.18 |
| t1271435 | 2 | library | 127.37 | 822.28 | 3.03 | 271.38 |
| t1271435 | 3 | library | 127.37 | 2,202.76 | 72.20 | 30.51 |

Analysis of the performance of these 20 improved PGA variants and their associated mutations revealed two classes of mutations that were generally associated with distinct performance properties of the enzymes (Table 5). One class of mutations (including, e.g., T181, M182, R185, N326, L362 and T482) was associated with an improved rate of D-amoxicillin formation, while a second set of mutations (including, e.g., T90, A189, W333, D380, H498, V563, and L770) was associated with improvement in the S:H ratio. Amino acid numbering for amino acid substitutions is relative to the sequence of the PGA from Achromobacter sp. CCM 4824 (SEQ ID NO: 1).

TABLE 5
Mutations within the 20 improved PGA variants

| | | Amoxicillin Synthesis |
|---|---|---|
| Strain | Mutations | Performance Group |
| t1268657 | M182N F330A | S:H improvement |
| t1268681 | F330A W333Y | Rate improvement |
| t1268686 | T90V A189V F330A W333I D380V | Rate improvement |
| t1270626 | T90V A189V F330A D380V | Rate improvement |
| | H498L V563Q | |
| t1270660 | M182L F330A T482P | S:H improvement |
| t1270676 | T90V A189V F330A W333I | Rate improvement |
| | D380I H498L | |
| t1270764 | M182T F330A L362F | S:H improvement |
| t1270788 | M182L R185F F330A T482P | S:H improvement |
| t1270829 | F330G | Rate improvement |
| t1270849 | M182I F330A | S:H improvement |
| t1270860 | R185F N326A F330A T482P | S:H improvement |
| t1270941 | F330A W333P | Rate improvement |
| t1270992 | F330A L770T | Rate improvement |
| t1271027 | T181M F330A | S:H improvement |
| t1271129 | F330A D380Q | Rate improvement |
| t1271132 | F330A W333I | Rate improvement |
| t1271162 | R185F F330A T482P | S:H improvement |
| t1271259 | F330A L770A | Rate improvement |
| t1271308 | T90V A189V F330A W333T | Rate improvement |
| | D380V H498S | |
| t1271435 | T181M M182F F330A L362F | S:H improvement |

TABLE 6
Mutations within the 20 improved PGA variants associated with improved
amoxicillin synthesis rate

| Strain | Mutations |
|---|---|
| t1268681 | F330A W333Y |
| t1268686 | T90V A189V F330A W333I D380V |
| t1270626 | T90V A189V F330A D380V H498L V563Q |
| t1270676 | T90V A189V F330A W333I D380I H498L |
| t1270829 | F330G |
| t1270941 | F330A W333P |
| t1270992 | F330A L770T |
| t1271129 | F330A D380Q |
| t1271132 | F330A W333I |
| t1271259 | F330A L770A |
| t1271308 | T90V A189V F330A W333T D380V H498S |

TABLE 7
Mutations within the 20 improved PGA variants associated with S:H ratio
improvement

| Strain | Mutations |
|---|---|
| t1268657 | M182N F330A |
| t1270660 | M182L F330A T482P |
| t1270764 | M182T F330A L362F |
| t1270788 | M182L R185F F330A T482P |
| t1270849 | M182I F330A |
| t1270860 | R185F N326A F330A T482P |
| t1271027 | T181M F330A |
| t1271162 | R185F F330A T482P |
| t1271435 | T181M M182F F330A L362F |

### D-Amoxicillin Synthesis Assay—Secondary Screen

Amoxicillin secondary screening was conducted in the same manner as primary screening described in Example 1, with modifications to the sampling time points, reaction buffer composition, and dilution of culture samples. 300 nL/well of thawed glycerol stocks of PGA variant E. coli transformants were dispensed into 30 μL/well of Teknova ZY media and sealed with AeraSeals. Samples were incubated at 20° C. for 40 hours, and diluted with 60 μL of TBS. At this stage strains that had been identified as particularly active during primary screening were further diluted by adding 7.5 μL of culture plus TBS to samples containing an inactive strain. All samples were then centrifuged for 10 minutes at 4° C. and 4000×g. The supernatant was aspirated and cell pellets were frozen at −80° C.

Pellets were thawed by shaking for 20 minutes at 30° C., and then lysed by the addition of 45 μL of lysis buffer. Lysate plates were sealed, shaken for 20 m at 1,000 rpm and 30° C. in an Infors Multitron HT incubator, and centrifuged for 10 s at 500 RCF. In parallel, 384 well microtiter plates were prepared containing reaction buffer and analytical standards. In addition to the same conditions used during primary screening, higher concentrations of substrate were also employed. For these reactions samples corresponding to enzyme variants being tested contained 54 μL of reaction buffer with 5.55 mM 6-aminopenicillanic acid, 5.55 mM hydroxyphenylglycine methyl ester, and 22.2 mM MOPS pH 6. Analytical standard samples contained 22.2 mM MOPS pH 6 and varying concentrations of amoxicillin and hydroxyphenylglycine, ranging from zero to 5.71 mM (amoxicillin) and from zero to 5.64 mM (hydroxyphenylglycine). Reactions were initiated by stamping 6 μL of lysate into the reaction plates. Samples were incubated at room temperature, and at 2, 4, 16 to 20, and 48 hours after the reaction was initiated samples were quenched and processed as described for the primary screen described in Example 1.

### Example 3: Functional Expression of Variant PGAs and Screening for Activity in Cephalexin Synthesis

As outlined in FIG. 2, during cephalexin synthesis, 7-ADCA and D-PGM are transformed enzymatically to cephalexin by a PGA. The PGA can synthesize the desired product cephalexin or it can hydrolyze the D-PGM to produce the undesired by-product D-PG. The ratio of the amount of desired “synthesis” product (cephalexin) to the amount of “hydrolysis” by-product (D-PG) is referred to in this disclosure as the synthesis: hydrolysis ratio or S:H ratio. The hydrolysis of cephalexin product to 7-ADCA and D-PG by PGA is an additional source of D-PG by-product. Optimizing the S:H ratio to increase the amount of cephalexin produced and reduce the amount of D-PG produced is beneficial in the commercial production of cephalexin. This Example relates to the engineering of a PGA from Achromobacter sp. CCM 4824 for improving the rate of cephalexin formation as well as improving the S:H ratio.

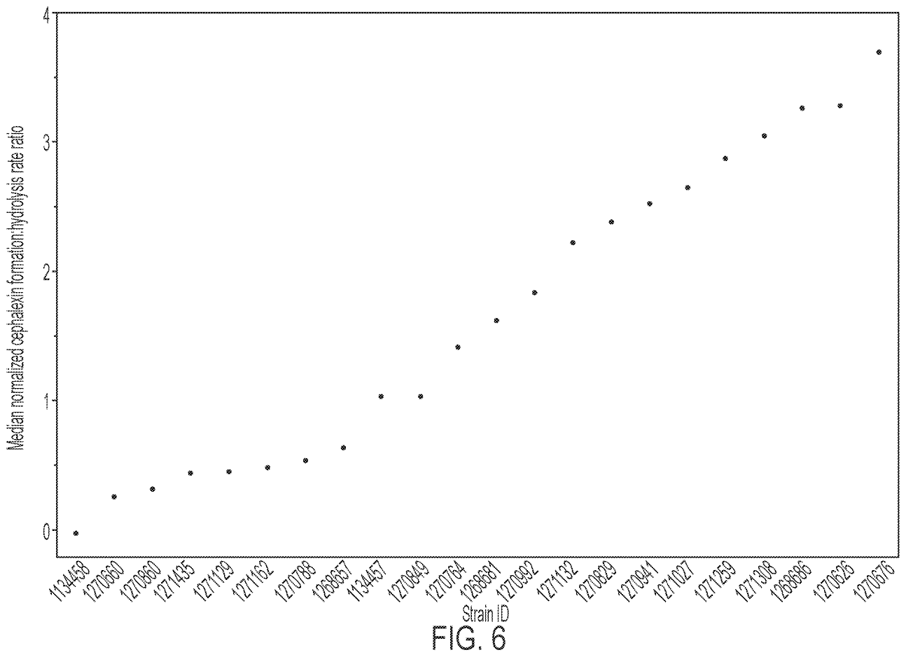

To determine whether the 20 PGA variants reported in Example 2 to have improved D-amoxicillin synthesis also exhibit improved cephalexin synthesis, a secondary screen was conducted that assessed the cephalexin formation rate and cephalexin hydrolysis rate as described below and shown in FIG. 6.

E. coli transformants expressing the PGA variants were tested for cephalexin formation and cephalexin hydrolysis by growing clonal expression cultures with biological replicates, followed by preparation of cell lysates, and feeding of the substrates, 7-ADCA and D-PGM under appropriate reaction conditions in vitro for the cephalexin formation assay or feeding the substrate cephalexin under appropriate reaction conditions in vitro for the cephalexin hydrolysis assay. Cephalexin concentrations in samples were determined using a spectrophotometric assay and comparing measured values to a standard curve of cephalexin standard. Average cephalexin formation rates and average cephalexin hydrolysis rates were normalized to the positive control with a value of 1. The median normalized cephalexin formation rate to normalized cephalexin hydrolysis rate ratio was calculated and used to assess performance of PGA variants against the positive and negative controls (Table 9).

TABLE 9
Performance of 20 PGA variants in cephalexin
synthesis and cephalexin hydrolysis

| | | cephalexin | cephalexin | cephalexin |
|---|---|---|---|---|
| | Sample | formation | consumption | S:H |
| Strain | Type | rate^{AN} | rate^{AN} | ratio^{MN} |
| t1270676 | library | 8.92 | 2.24 | 3.69 |
| t1270626 | library | 1.68 | 0.52 | 3.28 |
| t1268686 | library | 8.48 | 2.65 | 3.26 |
| t1271308 | library | 11 | 3.63 | 3.04 |
| t1271259 | library | 4.37 | 1.54 | 2.87 |
| t1271027 | library | 5.59 | 2.14 | 2.64 |
| t1270941 | library | 6.01 | 2.31 | 2.52 |
| t1270829 | library | 6.04 | 2.6 | 2.38 |
| t1271132 | library | 6.62 | 2.97 | 2.22 |
| t1270992 | library | 1.7 | 0.82 | 1.83 |
| t1268681 | library | 1.83 | 1.03 | 1.62 |
| t1270764 | library | 0.75 | 0.5 | 1.41 |
| t1270849 | library | 0.52 | 0.53 | 1.03 |
| t1134457 | positive | 1 | 1 | 1.03 |
| | control | | | |
| t1268657 | library | 0.33 | 0.42 | 0.63 |
| t1270788 | library | 0.33 | 0.64 | 0.53 |
| t1271162 | library | 0.45 | 0.85 | 0.48 |
| t1271129 | library | 0.24 | 0.48 | 0.45 |
| 11271435 | library | 0.42 | 0.35 | 0.44 |
| t1270860 | library | 0.52 | 1.18 | 0.31 |
| t1270660 | library | 0.07 | 0.26 | 0.25 |
| 11134458 | negative | 0.01 | −0.07 | −0.03 |
| | control | | | |

^{AN}Average normalized values;
^{MN}Median normalized values

Of the 20 PGA variants tested, 12 had cephalexin formation: hydrolysis ratios that were improved compared to the positive control PGA expressed by strain t1134457. Combined 5 analysis of the mutations and cephalexin performance identified residue W333 (relative to the sequence of SEQ ID NO: 1) as a position strongly associated with an improved cephalexin formation: hydrolysis ratio.

### Cephalexin Synthesis and Hydrolysis Assays

Pellets were thawed by shaking for 20 minutes at 30° C., and then lysed by the addition of 45 μL of lysis buffer. Lysate plates were sealed, shaken for 20 m at 1,000 rpm and 30° C. in an Infors Multitron HT incubator, and centrifuged for 10 s at 500 RCF. In parallel, 384 well microtiter plates were prepared containing reaction buffer and analytical standards. Two reaction plates were prepared. In one, reaction samples contained 120 μL of reaction buffer with 10.4 mM 7-ADCA, 10.4 mM D-PGM, and 104.2 mM HEPES, pH 7.2. In the other, reaction samples contained 11.11 mM cephalexin and 104.2 mM HEPES, pH 7.2. Analytical standard samples contained 104.2 mM HEPES pH 7.2 and varying concentrations of cephalosporin, ranging from zero to 10.4 mM, and were prepared by diluting cephalosporin stocks into equivalent concentration stocks of 7-ADCA. Reactions were initiated by stamping 5 μL of lysate into the reaction plates. Samples were incubated at room temperature, and at 9 minutes after initiation (for the plate containing 7-ADCA and D-PGM) or 22 hours (for the plate containing cephalexin) 20 μL of reaction sample was removed and added to 40 μL of 750 mM sodium hydroxide. Plates were incubated for 1 hour and then absorbance at 450 nm was measured in a plate reader.

TABLE 10
Sequences of alpha and beta subunits of
PGAs described in Examples 1-4

| | | Nucleotide | Protein |
|---|---|---|---|
| Strain ID | Sequence Type | SEQ ID NO: | SEQ ID NO: |
| t1268657 | Alpha Subunit | 43 | 3 |
| t1268657 | Beta Subunit | 63 | 23 |
| t1270626 | Alpha Subunit | 44 | 4 |
| t1270626 | Beta Subunit | 64 | 24 |
| t1270992 | Alpha Subunit | 45 | 5 |
| t1270992 | Beta Subunit | 65 | 25 |
| t1270849 | Alpha Subunit | 46 | 6 |
| t1270849 | Beta Subunit | 66 | 23 |
| t1270941 | Alpha Subunit | 45 | 5 |
| t1270941 | Beta Subunit | 67 | 27 |
| t1271435 | Alpha Subunit | 48 | 8 |
| t1271435 | Beta Subunit | 68 | 28 |
| t1268681 | Alpha Subunit | 45 | 5 |
| t1268681 | Beta Subunit | 69 | 29 |
| t1270788 | Alpha Subunit | 50 | 10 |
| t1270788 | Beta Subunit | 70 | 30 |
| t1270676 | Alpha Subunit | 51 | 4 |
| t1270676 | Beta Subunit | 71 | 31 |
| t1271132 | Alpha Subunit | 45 | 5 |
| t1271132 | Beta Subunit | 72 | 32 |
| t1271027 | Alpha Subunit | 53 | 13 |
| t1271027 | Beta Subunit | 66 | 23 |
| t1271129 | Alpha Subunit | 45 | 5 |
| t1271129 | Beta Subunit | 74 | 34 |
| t1268686 | Alpha Subunit | 51 | 4 |
| t1268686 | Beta Subunit | 75 | 35 |
| t1271308 | Alpha Subunit | 44 | 4 |
| t1271308 | Beta Subunit | 76 | 36 |
| t1271259 | Alpha Subunit | 45 | 5 |
| t1271259 | Beta Subunit | 77 | 37 |
| t1270860 | Alpha Subunit | 58 | 18 |
| t1270860 | Beta Subunit | 78 | 38 |
| t1270829 | Alpha Subunit | 45 | 5 |
| t1270829 | Beta Subunit | 79 | 39 |
| t1270764 | Alpha Subunit | 60 | 20 |
| t1270764 | Beta Subunit | 80 | 28 |
| t1270660 | Alpha Subunit | 61 | 21 |
| t1270660 | Beta Subunit | 81 | 30 |
| t1271162 | Alpha Subunit | 58 | 18 |
| t1271162 | Beta Subunit | 81 | 30 |

For the various PGAs described in Table 10, in some embodiments, the amino acid sequence of the linker is or comprises SEQ ID NO: 87. It should be appreciated that other linker sequences would also be compatible.

It should be appreciated that sequences disclosed in this application may or may not contain signal peptides. The sequences disclosed in this application encompass versions with or without signal peptides. It should also be understood that amino acid sequences disclosed in this application may be depicted with or without a start codon (M). The sequences disclosed in this application encompass versions with or without start codons. Accordingly, in some instances amino acid numbering may correspond to amino acid sequences containing a signal peptide and/or a start codon, while in other instances, amino acid numbering may correspond to amino acid sequences that do not contain a signal peptide and/or a start codon. It should also be understood that sequences disclosed in this application may be depicted with or without a stop codon. The sequences disclosed in this application encompass versions with or without stop codons, wherein: a stop codon (if present) can be removed or replaced by any other stop codon; or a stop codon (if not present) can be appended to the 3′-end of the sequence.

### EQUIVALENTS

Those skilled in the art will recognize or be able to ascertain using no more than routine experimentation, many equivalents to the specific embodiments of the invention described in the present application. Such equivalents are intended to be encompassed by the following claims.

All references, including patent documents, are incorporated by reference in their entirety.

What is claimed is:

1. A penicillin G acylase (PGA), wherein the PGA comprises amino acid substitutions relative to the sequence of SEQ ID NO: 1 selected from the group consisting of:
(i) F330A and W333I;
(ii) M182I and F330A;
(iii) T90V, A189V, F330A, W333I and D380V;
(iv) T90V, A189V, F330A, D380V, H498L and V563Q;
(v) M182L, F330A and T482P;
(vi) T90V, A189V, F330A, W333I, D380I and H498L;
(vii) M182T, F330A and L362F;
(viii) M182L, R185F, F330A and T482P;
(ix) F330G;
(x) F330A and W333Y;
(xi) R185F, N326A, F330A and T482P;
(xii) F330A and W333P;
(xiii) F330A and L770T;
(xiv) T181M and F330A;
(xv) F330A and D380Q;
(xvi) M182N and F330A;
(xvii) R185F, F330A and T482P;
(xviii) F330A and L770A;
(xix) T90V, A189V, F330A, W333T, D380V and H498S; and
(xx) T181M, M182F, F330A and L362F.

2. (canceled)

3. The PGA of claim 1, wherein the PGA comprises F330A and W333I amino acid substitutions relative to the sequence of SEQ ID NO: 1, or M182I and F330A amino acid substitutions relative to the sequence of SEQ ID NO: 1.

4. (canceled)

5. The PGA of claim 1, wherein the PGA comprises a sequence that is at least 70% identical to SEQ ID NO: 1 or SEQ ID NO: 83.

6. The PGA of claim 1, wherein the PGA comprises an alpha subunit and a beta subunit, wherein the sequence of the alpha subunit is at least 70% identical the sequence of any one of SEQ ID NOs: 3-6, 8, 10, 13, 18, 20, 21, 121, 123, 125, 127, 129, 131, or 133.

7. (canceled)

8. The PGA of claim 1, wherein the sequence of the beta subunit is at least 70% identical to the sequence of any one of SEQ ID NOs: 23-25, 27-32, 34-39, or 88.

9. (canceled)

10. The PGA of claim 1, wherein the PGA further comprises a signal peptide.

11. The PGA of claim 1, wherein the PGA comprises an alpha subunit and a beta subunit and further comprises a spacer interposed between the alpha subunit and the beta subunit.

12-16. (canceled)

17. The PGA of claim 1, wherein the PGA comprises an alpha subunit and a beta subunit, wherein the sequence of the alpha subunit is or comprises SEQ ID NO: 3 and the sequence of the beta subunit is or comprises SEQ ID NO: 23.

18. The PGA of claim 1, wherein the PGA comprises an alpha subunit and a beta subunit, wherein the sequence of the alpha subunit is or comprises SEQ ID NO: 4 and the sequence of the beta subunit is or comprises SEQ ID NO: 24, SEQ ID NO: 31, SEQ ID NO: 35, or SEQ ID NO: 36.

19. The PGA of claim 1, wherein the PGA comprises an alpha subunit and a beta subunit, wherein the sequence of the alpha subunit is or comprises SEQ ID NO: 5 and the sequence of the beta subunit is or comprises SEQ ID NO: 25, SEQ ID NO: 27, SEQ ID NO: 29, SEQ ID NO: 32, SEQ ID NO: 34, SEQ ID NO: 37, or SEQ ID NO: 39.

20. The PGA of claim 1, wherein the PGA comprises an alpha subunit and a beta subunit, wherein the sequence of the alpha subunit is or comprises SEQ ID NO: 6 and the sequence of the beta subunit is or comprises SEQ ID NO: 23.

21. (canceled)

22. The PGA of claim 1, wherein the PGA comprises an alpha subunit and a beta subunit, wherein the sequence of the alpha subunit is or comprises SEQ ID NO: 8 and the sequence of the beta subunit is or comprises SEQ ID NO: 28.

23. (canceled)

24. The PGA of claim 1, wherein the PGA comprises an alpha subunit and a beta subunit, wherein the sequence of the alpha subunit is or comprises SEQ ID NO: 10 and the sequence of the beta subunit is or comprises SEQ ID NO: 30.

25-26. (canceled)

27. The PGA of claim 1, wherein the PGA comprises an alpha subunit and a beta subunit, wherein the sequence of the alpha subunit is or comprises SEQ ID NO: 13 and the sequence of the beta subunit is or comprises SEQ ID NO: 23.

28-31. (canceled)

32. The PGA of claim 1, wherein the PGA comprises an alpha subunit and a beta subunit, wherein the sequence of the alpha subunit is or comprises SEQ ID NO: 18 and the sequence of the beta subunit is or comprises SEQ ID NO: 38 or SEQ ID NO: 30.

33. (canceled)

34. The PGA of claim 1, wherein the PGA comprises an alpha subunit and a beta subunit, wherein the sequence of the alpha subunit is or comprises SEQ ID NO: 20 and the sequence of the beta subunit is or comprises SEQ ID NO: 28.

35. The PGA of claim 1, wherein the PGA comprises an alpha subunit and a beta subunit, wherein the sequence of the alpha subunit is or comprises SEQ ID NO: 21 and the sequence of the beta subunit is or comprises SEQ ID NO: 30.

36. (canceled)

37. The PGA of claim 11, wherein the spacer sequence comprises a sequence that is at least 80% identical to any one of SEQ ID NOs: 87, 95, 97, or 99.

38-63. (canceled)

64. A host cell that comprises the PGA of claim 1.

65-74. (canceled)

75. A method of converting 6-aminopenicillanic acid (6-APA) and D-4-hydroxyphenylglucine methyl ester (D-HPGM) to D-amoxicillin, comprising contacting 6-APA and D-HPGM with a penicillin G acylase (PGA), wherein the PGA comprises amino acid substitution relative to the sequence of SEQ ID NO: 1 selected from the group consisting of:
(i) F330A and W333I;
(ii) M182I and F330A;
(iii) T90V, A189V, F330A, W333I and D380V;
(iv) T90V, A189V, F330A, D380V, H498L and V563Q;
(v) M182L, F330A and T482P;
(vi) T90V, A189V, F330A, W333I, D380I and H498L;
(vii) M182T, F330A and L362F;
(viii) M182L, R185F, F330A and T482P;
(ix) F330G;
(x) F330A and W333Y;
(xi) R185F, N326A, F330A and T482P;
(xii) F330A and W333P;
(xiii) F330A and L770T;
(xiv) T181M and F330A;
(XV) F330A and D380Q;
(xvi) M182N and F330A;
(xvii) R185F, F330A and T482P;
(xviii) F330A and L770A;
(xix) T90V, A189V, F330A, W333T, D380V and H498S; and
(xx) T181M, M182F, F330A and L362F.

76-78. (canceled)

79. A method of converting 7-aminodeacetoxycephalosporanic acid (7-ADCA) and D-phenylglycine methyl ester (D-PGM) to cephalexin, comprising contacting 7-ADCA and D-PGM with a penicillin G acylase (PGA), wherein the PGA comprises amino acid substitution relative to the sequence of SEQ ID NO: 1 selected from the group consisting of:
(i) F330A and W333I;
(ii) M182I and F330A;
(iii) T90V, A189V, F330A, W333I and D380V;
(iv) T90V, A189V, F330A, D380V, H498L and V563Q;
(v) M182L, F330A and T482P;
(vi) T90V, A189V, F330A, W333I, D380I and H498L;
(vii) M182T, F330A and L362F;
(viii) M182L, R185F, F330A and T482P;
(ix) F330G;
(x) F330A and W333Y;
(xi) R185F, N326A, F330A and T482P;
(xii) F330A and W333P;
(xiii) F330A and L770T;
(xiv) T181M and F330A;
(XV) F330A and D380Q;
(xvi) M182N and F330A;
(xvii) R185F, F330A and T482P;
(xviii) F330A and L770A;
(xix) T90V, A189V, F330A, W333T, D380V and H498S; and
(xx) T181M, M182F, F330A and L362F.

80-87. (canceled)


SEQUENCE LISTING (134 sequences)

SEQ ID NO: 1 - protein, 863 aa, Achromobacter sp.
MKQQWLSAALLAASSCLPAMAAQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADPLAYALPRYDGTPPMLERVVRDPATRGVVDGAPATLRAQLAAQYAQSGQPGIAGFPTTSNMWIVGRDHAKDARSILLNGPQFGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 2 - DNA, 2592 bp, Achromobacter sp.
  source 1..2592: mol_type = genomic DNA
atgaagcagcaatggttgtcggccgccctgttggcggccagttcgtgcctgcccgcgatggcggcgcagccggtggcgccagccgccggccagacgtccgaggcggttgcggcacggccccaaaccgccgatggcaaggtcacgatccggcgcgatgcctacggcatgccgcatgtctatgccgacacggtgtacggcatcttctacggctacggctacgcggtggcgcaggaccggctgttccagatggagatggcgcggcgcagcacccagggccgggtggccgaggtgctgggcgcctcgatggtgggcttcgacaagtcgatccgcgccaatttctcgcccgagcgcatccagcgccagttggcggcgctgccggccgccgaccgccaggtgctggacggctacgcggctggcatgaacgcctggctggcgcgggtgcgggcccagccgggccaactgatgcccaaggaattcaatgacctgggtttcgcgccggccgactggaccgcctacgacgtggcgatgatcttcgtcggcaccatggccaaccgcttttcggacgccaacagcgagatcgacaacctggcgctgctgacggcgttgaaggaccggcatggcgccgccgatgccatgcgcatcttcaaccagttgcgctggctgaccgacagccgcgcgccgaccacggtgccggccgaagcgggcagctaccagccgccggtgttccagccggacggcgcggacccgctggcctacgcgctgccgcgctacgacggcacgccgccgatgctcgagcgggtggtgcgcgacccggccacgcggggcgtggtcgacggcgcgccggcgacgctgcgggcgcaactggccgcccaatacgcgcaatcgggccagcccggcatcgccggctttccgaccaccagcaatatgtggatcgtgggccgcgaccacgccaaggacgcgcgctcgatcctgctgaacggcccgcagttcggctggtggaatccggcctatacctacggcatcggcttgcacggcgccggcttcgacgtggtcggcaacacgccgttcgcctatcccagcattctgttcggccacaatgcacacgtgacgtggggttcgaccgcgggcttcggcgatgacgtcgacatctttgccgaaaagctcgatcccgccgaccgcacgcgctatttccacgacggccaatggaagacgctggaaaagcgcaccgacctgatcctggtgaaggacgcggcgccagtgacgctggacgtgtaccgcagcgtgcatggcctgatcgtcaagttcgacgacgcgcagcacgtggcctacgccaaggcgcgcgcctgggaaggctatgaactgcaatcgctgatggcctggacccgcaagacgcaatcggccaactgggaacagtggaaggcgcaggcggcgcgccatgcgctgaccatcaactggtactacgccgacgaccgcggcaacattggctacgcgcacacgggcttctatcccaggcgccgtccgggccacgatccgcgcctgccggtgcccggcaccggcgagatggactggctgggcctgctgccgttctctaccaatccgcaggtctacaacccgcgccagggcttcatcgccaactggaacaaccagccgatgcgcggctacccgtccaccgacctgttcgccatcgtctggggccaggccgaccgctacgccgagatcgagacgcgcctgaaggccatgaccgcgaacggaggcaaggtcagcgcgcagcagatgtgggacctgatccgcaccaccagctacgccgacgtcaaccgccgtcatttcctgccgttcctgcaacgcgcggtgcaagggctgccggcggatgatccgcgcgtgcgcctggtggccggcctggcggcctgggacggcatgatgaccagcgagcgccaaccgggttacttcgacaacgccggcccggcggtcatggacgcgtggctgcgcgccatgctgcggcgcacgctggccgacgagatgccggccgacttcttcaagtggtacagcgccaccggctacccgacaccgcaggcgccggccaccggttcgctcaacctgaccaccggcgtcaaggtgctgttcaacgccctggccgggcccgaggctggcgtgccgcagcgctatgacttcttcaacggcgcgcgcgccgacgacgtcatcctcgcggcgctggacgatgcgctggcggcgctgcgccaggcctatggccaggatccggcggcatggaagatcccggcgccgccgatggtgttcgcgcccaagaacttcctgggcgtgccgcaggccgacgccaaggcggtgctgtgctatcgggccacgcagaaccgcggcaccgagaacaacatgacggtgttcgacggtaaatcggtgcgcgcggtggatgtggtggcgccggggcagagcggcttcgtcgccccggacggcacgccgtcgccgcacacccgcgaccagttcgacctgtacaacaccttcggcagcaaacgggtgtggttcacggccgatgaggtgcggcgcaacgctacgtcggaagagacgttgcgctacccgcggtaa

SEQ ID NO: 3 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTNANRFSDANSEIDNLALLTALKDRHGAADAMRIFNQLRWLTDSRAPTTVPAEAGSYQPPVFQPDGADPLAYALPRYDGTPPMLERVV

SEQ ID NO: 4 - protein, 247 aa, synthetic construct
AQPVAPAAGQTSEAVAARPQTADGKVTIRRDAYGMPHVYADTVYGIFYGYGYAVAQDRLFQMEMARRSVQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDVNSEIDNLALLTALKDRHGAADAMRIFNQLRWLTDSRAPTTVPAEAGSYQPPVFQPDGADPLAYALPRYDGTPPMLERVV

SEQ ID NO: 5 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADPLAYALPRYDGTPPMLERVV

SEQ ID NO: 6 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTIANRFSDANSEIDNLALLTALKDRHGAADAMRIFNQLRWLTDSRAPTTVPAEAGSYQPPVFQPDGADPLAYALPRYDGTPPMLERVV

SEQ ID NO: 7 - skipped

SEQ ID NO: 8 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGMFANRFSDANSEIDNLALLTALKDRHGAADAMRIFNQLRWLTDSRAPTTVPAEAGSYQPPVFQPDGADPLAYALPRYDGTPPMLERVV

SEQ ID NO: 9 - skipped

SEQ ID NO: 10 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTLANFFSDANSEIDNLALLTALKDRHGAADAMRIFNQLRWLTDSRAPTTVPAEAGSYQPPVFQPDGADPLAYALPRYDGTPPMLERVV

SEQ ID NO: 11-12 - skipped (2 entries)

SEQ ID NO: 13 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGMMANRFSDANSEIDNLALLTALKDRHGAADAMRIFNQLRWLTDSRAPTTVPAEAGSYQPPVFQPDGADPLAYALPRYDGTPPMLERVV

SEQ ID NO: 14-17 - skipped (4 entries)

SEQ ID NO: 18 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANFFSDANSEIDNLALLTALKDRHGAADAMRIFNQLRWLTDSRAPTTVPAEAGSYQPPVFQPDGADPLAYALPRYDGTPPMLERVV

SEQ ID NO: 19 - skipped

SEQ ID NO: 20 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTTANRFSDANSEIDNLALLTALKDRHGAADAMRIFNQLRWLTDSRAPTTVPAEAGSYQPPVFQPDGADPLAYALPRYDGTPPMLERVV

SEQ ID NO: 21 - protein, 247 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTLANRFSDANSEIDNLALLTALKDRHGAADAMRIFNQLRWLTDSRAPTTVPAEAGSYQPPVFQPDGADPLAYALPRYDGTPPMLERVV

SEQ ID NO: 22 - skipped

SEQ ID NO: 23 - protein, 557 aa, synthetic construct
SNMWIVGRDHAKDARSILLNGPQAGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 24 - protein, 557 aa, synthetic construct
SNMWIVGRDHAKDARSILLNGPQAGWWNPAYTYGIGLHGAGFDVVGNTPFAYPSILFGHNAHVTWGSTAGFGDVVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYALTGFYPRRRPGHDPRLPVPGTGEMDWLGLLPFSTNPQVYNPRQGFIANWNNQPMRGYPSTDLFAIQWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 25 - protein, 557 aa, synthetic construct
SNMWIVGRDHAKDARSILLNGPQAGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFTGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 26 - skipped

SEQ ID NO: 27 - protein, 557 aa, synthetic construct
SNMWIVGRDHAKDARSILLNGPQAGWPNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 28 - protein, 557 aa, synthetic construct
SNMWIVGRDHAKDARSILLNGPQAGWWNPAYTYGIGLHGAGFDVVGNTPFAYPSIFFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 29 - protein, 557 aa, synthetic construct
SNMWIVGRDHAKDARSILLNGPQAGWYNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 30 - protein, 557 aa, synthetic construct
SNMWIVGRDHAKDARSILLNGPQAGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALPINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 31 - protein, 557 aa, synthetic construct
SNMWIVGRDHAKDARSILLNGPQAGWINPAYTYGIGLHGAGFDVVGNTPFAYPSILFGHNAHVTWGSTAGFGDIVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYALTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 32 - protein, 557 aa, synthetic construct
SNMWIVGRDHAKDARSILLNGPQAGWINPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 33 - skipped

SEQ ID NO: 34 - protein, 557 aa, synthetic construct
SNMWIVGRDHAKDARSILLNGPQAGWWNPAYTYGIGLHGAGFDVVGNTPFAYPSILFGHNAHVTWGSTAGFGDQVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 35 - protein, 557 aa, synthetic construct
SNMWIVGRDHAKDARSILLNGPQAGWINPAYTYGIGLHGAGFDVVGNTPFAYPSILFGHNAHVTWGSTAGFGDVVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 36 - protein, 557 aa, synthetic construct
SNMWIVGRDHAKDARSILLNGPQAGWTNPAYTYGIGLHGAGFDVVGNTPFAYPSILFGHNAHVTWGSTAGFGDVVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYASTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 37 - protein, 557 aa, synthetic construct
SNMWIVGRDHAKDARSILLNGPQAGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFAGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 38 - protein, 557 aa, synthetic construct
SNMWIVGRDHAKDARSILLAGPQAGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALPINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 39 - protein, 557 aa, synthetic construct
SNMWIVGRDHAKDARSILLNGPQGGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 40-42 - skipped (3 entries)

SEQ ID NO: 43 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccaacgcaaaccgtttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 44 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccgtacaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccatggcaaaccgtttctccgatgtgaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 45 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccatggcaaaccgtttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 46 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccatcgcaaaccgtttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 47 - skipped

SEQ ID NO: 48 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtatgtttgcaaaccgtttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 49 - skipped

SEQ ID NO: 50 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccctggcaaacttcttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 51 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccgttcaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccatggcaaaccgtttctccgatgttaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 52 - skipped

SEQ ID NO: 53 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtatgatggcaaaccgtttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 54-57 - skipped (4 entries)

SEQ ID NO: 58 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccatggcaaacttcttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 59 - skipped

SEQ ID NO: 60 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccactgcaaaccgtttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 61 - DNA, 741 bp, synthetic construct
  source 1..741: mol_type = other DNA
gctcagcctgttgctccggcagctggccagaccagcgaagcggtggcggcgcgtccgcagaccgcggacggtaaggttactatccgtcgcgacgcgtatggcatgccccacgtatatgccgacactgtctacggcattttttacggttacggctatgccgtggcgcaagatcgccttttccagatggaaatggctcgccgttccacccaaggtcgcgtggcagaagtgctgggcgcttctatggttggtttcgacaaaagcatccgcgctaacttttcccctgaacgcatccagcgccagctggcagctctgccagcagcagaccgtcaggtactggacggttacgcggctggtatgaatgcatggctggcacgtgtacgcgcgcagccaggtcagctgatgcctaaagaatttaacgatctgggtttcgctccggcagattggaccgcttatgacgttgctatgattttcgtgggtaccctggcaaaccgtttctccgatgccaactctgagatcgataacttggcactgctgactgcactgaaagaccgtcatggcgctgctgacgctatgcgcattttcaaccaactccgttggctgacggactcccgtgctcctaccactgtaccagctgaagctggctcttaccagccgccggtatttcaacccgacggtgcggatccgctggcgtacgctcttcctagatacgatggcaccccaccgatgctggaacgtgtggtt

SEQ ID NO: 62 - skipped

SEQ ID NO: 63 - DNA, 1674 bp, synthetic construct
  source 1..1674: mol_type = other DNA
agcaatatgtggatcgttggtcgtgaccacgcgaaagacgcgcgtagtatcctgctgaacggtccgcaggctggttggtggaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 64 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcaggttggtggaatccggcttatacctatgggattggcctccatggtgcaggctttgacgtggttggcaacactccgtttgcttacccctctatcctgttcggccataacgctcacgtaacctggggctctacggctggtttcggcgatgtggtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgccctgactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatccaatggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 65 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcgggttggtggaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcaccggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 66 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcgggttggtggaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 67 - DNA, 1674 bp, synthetic construct
  source 1..1674: mol_type = other DNA
agcaatatgtggatcgttggtcgtgaccacgcgaaagacgcgcgtagtatcctgctgaacggtccgcaggccggttggccgaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 68 - DNA, 1674 bp, synthetic construct
  source 1..1674: mol_type = other DNA
agcaatatgtggatcgttggtcgtgaccacgcgaaagacgcgcgtagtatcctgctgaacggtccgcaggctggttggtggaatccggcttatacctatgggattggcctccatggtgcaggctttgacgtggttggcaacactccgtttgcttacccctctatcttcttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 69 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcgggttggtataatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 70 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcaggttggtggaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcccgattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 71 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcaggttggatcaatccggcttatacctatgggattggcctccatggtgcaggctttgacgtggttggcaacactccgtttgcttacccctctatcctgttcggccataacgctcacgtaacctggggctctacggctggtttcggcgatatcgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgccctgactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 72 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcgggttggatcaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 73 - skipped

SEQ ID NO: 74 - DNA, 1674 bp, synthetic construct
  source 1..1674: mol_type = other DNA
agcaatatgtggatcgttggtcgtgaccacgcgaaagacgcgcgtagtatcctgctgaacggtccgcaggccggttggtggaatccggcttatacctatgggattggcctccatggtgcaggctttgacgtggttggcaacactccgtttgcttacccctctatcctgttcggccataacgctcacgtaacctggggctctacggctggtttcggcgatcaagtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 75 - DNA, 1674 bp, synthetic construct
  source 1..1674: mol_type = other DNA
agcaatatgtggatcgttggtcgtgaccacgcgaaagacgcgcgtagtatcctgctgaacggtccgcaggccggttggatcaatccggcttatacctatgggattggcctccatggtgcaggctttgacgtggttggcaacactccgtttgcttacccctctatcctgttcggccataacgctcacgtaacctggggctctacggctggtttcggcgatgtggtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 76 - DNA, 1674 bp, synthetic construct
  source 1..1674: mol_type = other DNA
agcaatatgtggatcgttggtcgtgaccacgcgaaagacgcgcgtagtatcctgctgaacggtccgcaggccggttggaccaatccggcttatacctatgggattggcctccatggtgcaggctttgacgtggttggcaacactccgtttgcttacccctctatcctgttcggccataacgctcacgtaacctggggctctacggctggtttcggcgatgtagtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgccagcactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 77 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcaggttggtggaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcgcgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 78 - DNA, 1674 bp, synthetic construct
  source 1..1674: mol_type = other DNA
agcaatatgtggatcgttggtcgtgaccacgcgaaagacgcgcgtagtatcctgctggcgggtccgcaggcaggttggtggaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcccgattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 79 - DNA, 1674 bp, synthetic construct
  source 1..1674: mol_type = other DNA
agcaatatgtggatcgttggtcgtgaccacgcgaaagacgcgcgtagtatcctgctgaacggtccgcagggcggttggtggaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 80 - DNA, 1674 bp, synthetic construct
  source 1..1674: mol_type = other DNA
agcaatatgtggatcgttggtcgtgaccacgcgaaagacgcgcgtagtatcctgctgaacggtccgcaggcaggttggtggaatccggcttatacctatgggattggcctccatggtgcaggctttgacgtggttggcaacactccgtttgcttacccctctatcttcttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcaccattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 81 - DNA, 1674 bp, synthetic construct
  source 1..1674: mol_type = other DNA
agcaatatgtggatcgttggtcgtgaccacgcgaaagacgcgcgtagtatcctgctgaacggtccgcaggccggttggtggaatccggcttatacctatgggattggcctccatggtgcaggctttgacgtggttggcaacactccgtttgcttacccctctatcctgttcggccataacgctcacgtaacctggggctctacggctggtttcggcgatgacgtggatatattcgccgagaaactggatcctgctgaccgtacccgctacttccatgacggccagtggaaaactctggagaaacgtaccgatctgatcctggtaaaagatgctgcgcccgtgaccctggatgtataccgctctgttcatggcctgattgtgaagttcgacgatgcgcagcacgtcgcgtatgcaaaggctcgcgcttgggaaggttatgagcttcagtctctgatggcatggactcgtaaaactcagtctgctaactgggaacaatggaaagctcaagcagctcgtcacgcgctcccgattaactggtattacgcagatgaccgcggcaacatcggttacgcccacactggtttctacccgcgccgccgtccgggccatgacccgcgtctgccagttcccggtacaggcgagatggattggctgggtctgctgccgttctctactaacccgcaggtatacaacccgcgtcagggcttcattgccaactggaacaatcagccgatgcgtggctacccttccacggacctattcgctatcgtttggggccaggctgatcgttatgctgaaatcgaaacgcgtctgaaagccatgactgccaacggcggcaaggtttctgctcagcagatgtgggacttaatccgcactaccagctatgccgacgtgaaccgacgccactttctgccgttcctgcaacgcgctgttcaagggctgccggctgacgatccgcgtgttcgcctggttgcaggtctggcagcctgggacggtatgatgacttctgaacgtcaaccgggttactttgacaacgcgggtccggcggttatggatgcatggcttcgtgcaatgctgcggcgtaccttagccgacgagatgcctgctgatttctttaaatggtatagcgccaccggttatccgaccccgcaagcacctgctactggtagcctgaacctgactaccggggttaaggttctctttaacgctctcgctggcccggaagctggggttcctcagcgctatgattttttcaacggtgctcgtgcagatgacgtgatcctggccgcgctggacgacgcgcttgcggcactgcgtcaagcttacgggcaggacccagccgcatggaaaatcccggctcccccgatggtcttcgcgccgaagaacttcctgggtgtaccgcaggccgatgccaaagctgtactgtgttaccgtgccactcaaaaccgtggcactgaaaacaacatgaccgtattcgacggtaagtctgttcgtgctgtagacgtcgttgcgccgggacagagcggctttgttgcaccagacggcactccttctcctcatacccgtgatcagttcgacctgtacaacacgttcggttctaaacgtgtttggttcaccgcggatgaggtacgccgcaacgcaaccagcgaagaaaccctgcgctacccgcgttaa

SEQ ID NO: 82 - skipped

SEQ ID NO: 83 - protein, 842 aa, Achromobacter sp.
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADPLAYALPRYDGTPPMLERVVRDPATRGVVDGAPATLRAQLAAQYAQSGQPGIAGFPTTSNMWIVGRDHAKDARSILLNGPQFGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 84 - DNA, 2529 bp, Achromobacter sp.
  source 1..2529: mol_type = genomic DNA
gcgcagccggtggcgccagccgccggccagacgtccgaggcggttgcggcacggccccaaaccgccgatggcaaggtcacgatccggcgcgatgcctacggcatgccgcatgtctatgccgacacggtgtacggcatcttctacggctacggctacgcggtggcgcaggaccggctgttccagatggagatggcgcggcgcagcacccagggccgggtggccgaggtgctgggcgcctcgatggtgggcttcgacaagtcgatccgcgccaatttctcgcccgagcgcatccagcgccagttggcggcgctgccggccgccgaccgccaggtgctggacggctacgcggctggcatgaacgcctggctggcgcgggtgcgggcccagccgggccaactgatgcccaaggaattcaatgacctgggtttcgcgccggccgactggaccgcctacgacgtggcgatgatcttcgtcggcaccatggccaaccgcttttcggacgccaacagcgagatcgacaacctggcgctgctgacggcgttgaaggaccggcatggcgccgccgatgccatgcgcatcttcaaccagttgcgctggctgaccgacagccgcgcgccgaccacggtgccggccgaagcgggcagctaccagccgccggtgttccagccggacggcgcggacccgctggcctacgcgctgccgcgctacgacggcacgccgccgatgctcgagcgggtggtgcgcgacccggccacgcggggcgtggtcgacggcgcgccggcgacgctgcgggcgcaactggccgcccaatacgcgcaatcgggccagcccggcatcgccggctttccgaccaccagcaatatgtggatcgtgggccgcgaccacgccaaggacgcgcgctcgatcctgctgaacggcccgcagttcggctggtggaatccggcctatacctacggcatcggcttgcacggcgccggcttcgacgtggtcggcaacacgccgttcgcctatcccagcattctgttcggccacaatgcacacgtgacgtggggttcgaccgcgggcttcggcgatgacgtcgacatctttgccgaaaagctcgatcccgccgaccgcacgcgctatttccacgacggccaatggaagacgctggaaaagcgcaccgacctgatcctggtgaaggacgcggcgccagtgacgctggacgtgtaccgcagcgtgcatggcctgatcgtcaagttcgacgacgcgcagcacgtggcctacgccaaggcgcgcgcctgggaaggctatgaactgcaatcgctgatggcctggacccgcaagacgcaatcggccaactgggaacagtggaaggcgcaggcggcgcgccatgcgctgaccatcaactggtactacgccgacgaccgcggcaacattggctacgcgcacacgggcttctatcccaggcgccgtccgggccacgatccgcgcctgccggtgcccggcaccggcgagatggactggctgggcctgctgccgttctctaccaatccgcaggtctacaacccgcgccagggcttcatcgccaactggaacaaccagccgatgcgcggctacccgtccaccgacctgttcgccatcgtctggggccaggccgaccgctacgccgagatcgagacgcgcctgaaggccatgaccgcgaacggaggcaaggtcagcgcgcagcagatgtgggacctgatccgcaccaccagctacgccgacgtcaaccgccgtcatttcctgccgttcctgcaacgcgcggtgcaagggctgccggcggatgatccgcgcgtgcgcctggtggccggcctggcggcctgggacggcatgatgaccagcgagcgccaaccgggttacttcgacaacgccggcccggcggtcatggacgcgtggctgcgcgccatgctgcggcgcacgctggccgacgagatgccggccgacttcttcaagtggtacagcgccaccggctacccgacaccgcaggcgccggccaccggttcgctcaacctgaccaccggcgtcaaggtgctgttcaacgccctggccgggcccgaggctggcgtgccgcagcgctatgacttcttcaacggcgcgcgcgccgacgacgtcatcctcgcggcgctggacgatgcgctggcggcgctgcgccaggcctatggccaggatccggcggcatggaagatcccggcgccgccgatggtgttcgcgcccaagaacttcctgggcgtgccgcaggccgacgccaaggcggtgctgtgctatcgggccacgcagaaccgcggcaccgagaacaacatgacggtgttcgacggtaaatcggtgcgcgcggtggatgtggtggcgccggggcagagcggcttcgtcgccccggacggcacgccgtcgccgcacacccgcgaccagttcgacctgtacaacaccttcggcagcaaacgggtgtggttcacggccgatgaggtgcggcgcaacgctacgtcggaagagacgttgcgctacccgcggtaa

SEQ ID NO: 85 - protein, 842 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADPLAYALPRYDGTPPMLERVVRDPATRGVVDGAPATLRAQLAAQYAQSGQPGIAGFPTTSNMWIVGRDHAKDARSILLNGPQAGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 86 - skipped

SEQ ID NO: 87 - protein, 38 aa, synthetic construct
RDPATRGVVDGAPATLRAQLAAQYAQSGQPGIAGFPTT

SEQ ID NO: 88 - protein, 557 aa, synthetic construct
SNMWIVGRDHAKDARSILLNGPQFGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 89 - DNA, 2529 bp, synthetic construct
  source 1..2529: mol_type = other DNA
gcgcaaccggtcgcaccagctgcgggtcagacctccgaggcggtagctgcccgtccgcaaaccgcagatggcaaggtcaccatccgtcgcgacgcgtatggcatgccacacgtttacgcggacaccgtctacggcattttctatggttacggctatgcggtcgcacaagatcgcttgttccagatggaaatggcgcgtcgttctacgcagggtcgcgttgcagaagtcctgggtgcttctatggtgggctttgacaagagcattcgtgcgaatttttctccggaacgcatccagcgccaactggctgcgctgcctgcagctgatcgccaggtcctggatggctacgcagcgggcatgaatgcttggctggcacgtgttcgtgctcaaccgggtcagctgatgccgaaagaatttaatgatctgggttttgctccggcggattggacggcgtatgatgtggcgatgatttttgtgggtaccatggctaaccgcttctcagacgccaacagcgagattgataacctggcactgctgaccgccctgaaagatcgtcacggtgcggctgatgcgatgcgcatcttcaaccaactgcgttggctgacggatagccgtgctccgacgacggttccggcagaggctggttcttaccagccgccggtttttcagccggatggtgcagatccgctggcatacgcgctgccgcgctatgacggtacgccgccgatgctggagcgtgtggtgcgcgatccggcaacccgtggtgtcgttgatggtgcaccggcaacgctgcgtgcacaactggcagcccagtacgcacagtccggtcagccgggtattgctggttttccgacgactagcaacatgtggattgttggccgtgaccatgccaaggatgcccgtagcatcctgctgaatggtccgcaggccggttggtggaatcctgcgtatacctacggcattggtctgcatggtgccggctttgacgtcgttggcaacaccccttttgcttacccgagcatcctgttcggtcataacgcccatgtgacttggggtagcactgccggtttcggcgatgacgttgatatcttcgccgagaaactggatcctgccgaccgtacccgttactttcacgacggccagtggaaaaccttggaaaagcgcactgatctgatcttggtgaaagacgcggcaccagtcacgctggacgtttaccgtagcgttcacggtctgattgttaagttcgatgatgcgcagcacgtcgcatacgcaaaggcccgtgcatgggaaggctacgagctgcagtccctgatggcctggacccgtaaaacccagagcgcgaattgggagcaatggaaagcgcaagcagctcgccacgcactgacgattaattggtattatgcagacgaccgcggcaacattggctatgcgcacaccggcttttatccacgtcgtcgtccgggtcacgatccgcgtttgccggttccgggcactggcgagatggactggctgggtctgttgccattttcgaccaacccgcaagtctataatccgcgtcagggctttattgccaattggaacaatcaaccgatgcgtggctaccctagcaccgatctgttcgctattgtttggggtcaagccgatcgctatgccgagatcgaaacccgcctgaaagccatgacggcgaacggtggtaaagtgagcgcgcaacagatgtgggacctgatccgcaccacgagctatgcggatgtcaatcgtcgccatttcttgcctttcctgcaacgtgccgtccaaggcctgccggcagatgatcctcgcgttcgcctggtagctggcctggcagcatgggacggcatgatgacgagcgaacgccaaccgggctacttcgataacgctggcccagccgttatggatgcttggctgcgtgcaatgctgcgccgcaccttggcagacgagatgccggcagacttcttcaaatggtacagcgcaacgggttatccgaccccgcaagctccagccactggtagcttgaatctgacgacgggcgttaaagtgttgtttaatgcgttggccggtcctgaggctggtgtgccgcagcgctacgacttcttcaatggtgcgcgtgcggatgacgtcattttagcggcattagacgacgccttggcggcactgcgtcaggcgtacggtcaggatccggcggcttggaagatccctgccccaccgatggtttttgccccgaaaaacttcttgggtgttccgcaagcggacgcgaaagctgttctgtgctatcgcgcgacgcaaaatcgtggtaccgagaataatatgaccgtgtttgacggcaagagcgtccgcgcagtggatgttgtcgctccgggccaatccggttttgtggccccggatggtaccccttctccgcatactcgtgaccagttcgacctgtacaacaccttcggcagcaagcgtgtctggttcaccgcggatgaggttcgtcgtaacgccacgagcgaagaaacgctgcgttatccgcgctaa

SEQ ID NO: 90 - protein, 12 aa, synthetic construct
SNMWIVGRDHAK

SEQ ID NO: 91 - protein, 12 aa, synthetic construct
SGQPGIAGFPTT

SEQ ID NO: 92 - protein, 59 aa, synthetic construct
DAMRIFNQLRWLTDSRAPTTVPAEAGSYQPPVFQPDGADPLAYALPRYDGTPPMLERVV

SEQ ID NO: 93 - skipped

SEQ ID NO: 94 - protein, 43 aa, synthetic construct
DAMRIFNQLRWLTDSRAPTTVPAEAGSYQPPVFQPDGADPLAY

SEQ ID NO: 95 - protein, 54 aa, synthetic construct
ALPRYDGTPPMLERVVRDPATRGVVDGAPATLRAQLAAQYAQSGQPGIAGFPTT

SEQ ID NO: 96 - protein, 40 aa, synthetic construct
DAMRIFNQLRWLTDSRAPTTVPAEAGSYQPPVFQPDGADP

SEQ ID NO: 97 - protein, 57 aa, synthetic construct
LAYALPRYDGTPPMLERVVRDPATRGVVDGAPATLRAQLAAQYAQSGQPGIAGFPTT

SEQ ID NO: 98 - protein, 6 aa, synthetic construct
DAMRIF

SEQ ID NO: 99 - protein, 91 aa, synthetic construct
NQLRWLTDSRAPTTVPAEAGSYQPPVFQPDGADPLAYALPRYDGTPPMLERVVRDPATRGVVDGAPATLRAQLAAQYAQSGQPGIAGFPTT

SEQ ID NO: 100-104 - skipped (5 entries)

SEQ ID NO: 105 - protein, 5 aa, synthetic construct
GGGGS

SEQ ID NO: 106 - protein, 5 aa, synthetic construct
GGGSS

SEQ ID NO: 107 - protein, 9 aa, synthetic construct
GGGGSGGGS

SEQ ID NO: 108 - protein, 16 aa, synthetic construct
GSGGGSGGGGSGGGGS

SEQ ID NO: 109 - protein, 50 aa, synthetic construct
  VARIANT 2..50: note = up to 49 copies of A may be deleted
AAAAAAAAAAAAAAAAAAAAAAAAAAAAAAAAAAAAAAAAAAAAAAAAAA

SEQ ID NO: 110 - protein, 100 aa, synthetic construct
  VARIANT 3..60: note = up to 49 copies of GA may be deleted
GAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGA

SEQ ID NO: 111 - protein, 250 aa, synthetic construct
  VARIANT 6..250: note = up 49 copies of GGGGS may be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 112 - skipped

SEQ ID NO: 113 - protein, 16 aa, synthetic construct
LLGAAAKGAAAKGSAA

SEQ ID NO: 114 - protein, 19 aa, synthetic construct
LLGGGGSGGGGSAAAGSAA

SEQ ID NO: 115 - protein, 25 aa, synthetic construct
MNHLVHHHHHHIEGRHMELGTLEGS

SEQ ID NO: 116 - protein, 9 aa, synthetic construct
MHHHHHHKH

SEQ ID NO: 117 - protein, 15 aa, synthetic construct
GLNDIFEAQKIEWHE

SEQ ID NO: 118 - protein, 21 aa, Achromobacter sp.
MKQQWLSAALLAASSCLPAMA

SEQ ID NO: 119 - protein, 40 aa, Achromobacter sp.
MKQQWLSAALLAASSCLPAMAAQPVAPAAGQTSEAVAARP

SEQ ID NO: 120 - protein, 823 aa, Achromobacter sp.
QTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADPLAYALPRYDGTPPMLERVVRDPATRGVVDGAPATLRAQLAAQYAQSGQPGIAGFPTTSNMWIVGRDHAKDARSILLNGPQFGWWNPAYTYGIGLHGAGFDVVGNTPFAYPSILFGHNAHVTWGSTAGFGDDVDIFAEKLDPADRTRYFHDGQWKTLEKRTDLILVKDAAPVTLDVYRSVHGLIVKFDDAQHVAYAKARAWEGYELQSLMAWTRKTQSANWEQWKAQAARHALTINWYYADDRGNIGYAHTGFYPRRRPGHDPRLPVPGTGEMDWLGLLPFSTNPQVYNPRQGFIANWNNQPMRGYPSTDLFAIVWGQADRYAEIETRLKAMTANGGKVSAQQMWDLIRTTSYADVNRRHFLPFLQRAVQGLPADDPRVRLVAGLAAWDGMMTSERQPGYFDNAGPAVMDAWLRAMLRRTLADEMPADFFKWYSATGYPTPQAPATGSLNLTTGVKVLFNALAGPEAGVPQRYDFFNGARADDVILAALDDALAALRQAYGQDPAAWKIPAPPMVFAPKNFLGVPQADAKAVLCYRATQNRGTENNMTVFDGKSVRAVDVVAPGQSGFVAPDGTPSPHTRDQFDLYNTFGSKRVWFTADEVRRNATSEETLRYPR

SEQ ID NO: 121 - protein, 231 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADPLAY

SEQ ID NO: 122 - skipped

SEQ ID NO: 123 - protein, 194 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIF

SEQ ID NO: 124 - skipped

SEQ ID NO: 125 - protein, 209 aa, synthetic construct
QTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADP

SEQ ID NO: 126 - skipped

SEQ ID NO: 127 - protein, 228 aa, synthetic construct
AQPVAPAAGQTSEAVAARPQTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADP

SEQ ID NO: 128 - skipped

SEQ ID NO: 129 - protein, 175 aa, synthetic construct
QTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIF

SEQ ID NO: 130 - skipped

SEQ ID NO: 131 - protein, 212 aa, synthetic construct
QTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADPLAY

SEQ ID NO: 132 - skipped

SEQ ID NO: 133 - protein, 228 aa, synthetic construct
QTADGKVTIRRDAYGMPHVYADTVYGIFYGYGYAVAQDRLFQMEMARRSTQGRVAEVLGASMVGFDKSIRANFSPERIQRQLAALPAADRQVLDGYAAGMNAWLARVRAQPGQLMPKEFNDLGFAPADWTAYDVAMIFVGTMANRFSDANSEIDNLALLTALKDRHGAADAMRIFNQLRWLTDSRAPTTVPAEAGSYQPPVFQPDGADPLAYALPRYDGTPPMLERVV

SEQ ID NO: 134 - skipped